(19) United States
(12) Patent Application Publication
(10) Pub. No.: US 2015/0037422 A1
(43) Pub. Date: Feb. 5, 2015

(54) COMPOSITIONS AND METHODS FOR OCULAR DELIVERY OF A THERAPEUTIC AGENT

(71) Applicant: Trustees of Tufts College, Medford, MA (US)

(72) Inventors: David L. Kaplan, Concord, MA (US); Michael L. Lovett, Peabody, MA (US); Xiaoqin Wang, Winchester, MA (US); Tuna Yucel, Medford, MA (US)

(21) Appl. No.: 14/380,333

(22) PCT Filed: Feb. 22, 2013
(86) PCT No.: PCT/US2013/027465

§ 371 Date: Aug. 21, 2014

Related U.S. Application Data
(60) Provisional application No. 61/601,924, filed on Feb. 22, 2012.

(51) Int. Cl. A61K 9/00; C07K 16/22; A61F 9/00; A61K 9/50

(52) U.S. Cl. CPC A61K 9/0048; A61K 9/5052; C07K 16/22; A61F 9/0017
USPC 424/491; 424/133.1; 604/187; 604/506

(57) ABSTRACT

Embodiments of various aspects described herein are directed to silk-based compositions for ocular delivery of at least one active agent, e.g., at least one therapeutic agent and methods of using the same. In some embodiments, the silk-based compositions can provide sustained release of at least one therapeutic agent to at least a portion of an eye. Thus, some embodiments of the silk-based compositions can be used for treatment of an ocular condition, e.g., age-related macular degeneration.

### CROSS REFERENCE TO RELATED APPLICATIONS

This application claims the benefit under 35 U.S.C. §119(e) of U.S. Provisional Application Nos. 61/601,924 filed Feb. 22, 2012, the content of which is incorporated herein by reference in its entirety.

### TECHNICAL FIELD

Embodiments of various aspects described herein relate to silk-based compositions for sustained delivery of at least one active agent, such as a therapeutic agent, to a target area, as well as methods of using the same. In some embodiments, the silk-based compositions and methods described herein can be used for ocular delivery of an active agent, e.g., to treat an ocular disease or disorder, e.g., age-related macular degeneration.

### BACKGROUND

Age-related macular degeneration (AMD), a degenerative disease characterized by the loss of the central vision, is the most common cause of blindness among people over 60 years of age. Approximately 14 million people worldwide are blind or severely visually impaired as a result of AMD. There are two forms of AMD: the ‘dry’ form is characterized by pigment disruption and small yellowish deposits called drusen; while the ‘wet’ form is characterized by the presence of fluid/blood at the back of the eye due to abnormal blood vessel formation. As the general population ages, the number of people afflicted with this disease will continue to grow unless more efficient and effective therapies are developed (Gehrs et al., Annals of Medicine 38 (2006), 450).

Current therapeutic approaches are tedious and hence, inconvenient for the patient. For example, photodynamic therapy with verteporfin (VISUDYNE®, Novartis) is a therapy in which the drug is intravenously infused before laser treatment in the eye is used to activate the verteporfin, resulting in damage to the local endothelium and vessel occlusion. This therapy typically requires several treatments (repeated every 3 months) and necessitates that the patient avoids exposure to light for 5 days post-treatment. Likewise, anti-vascular endothelial growth factor (VEGF) therapy requires repeated intravitreal injections (e.g., every 6 weeks with pegaptanib (MACUGEN®, Eyetech); each month with ranibizumab (LUCENTIS®, Genentech), each month with bevacizumab (AVASTIN®, Genentech)) or every 8 weeks following 3 initial doses administered every 4 weeks with aflibercept (EYLEA® (VEGF Trap-Eye), Regeneron)). Other options for sustained therapeutic delivery in ophthalmic indications include the Surmodics I-VATION™ TA intravitreal implant system which anchors to the sclera and controls release based on a poly(D,L-lactic-co-glycolic)acid (PLGA)-based polymer system. However, organic solvents and high temperatures are generally used for processing PLGA and hydrolytic degradation byproducts of PLGA are generally acids, which may cause inflammation and degradation of the active ingredient. Thus, there is a need for improved pharmaceutical compositions for ocular administrations and/or therapeutic interventions that can provide sustained delivery of therapeutic agent(s), e.g., anti-angiogenic agent(s), with greater patient comfort and thus greater patient compliance.

### SUMMARY

Embodiments of various aspects provided herein relate to silk-based compositions, delivery devices, kits and methods for sustained delivery of one or more therapeutic agents and uses thereof. Some embodiments of the silk-based compositions can be processed in completely aqueous based solvents, and can thus avoid or minimize the use of organic solvents or any harsh chemicals that can pose biocompatibility problems with any therapeutic agent(s) loaded therein. Generally, the silk-based composition described herein comprises a therapeutic agent dispersed or encapsulated in a silk matrix. In some embodiments, the silk-based compositions, delivery devices, and kits are formulated for ocular administration, which can then be used for ocular delivery of at least one therapeutic agent and/or treatment of an ocular condition.

In particular, the inventors have demonstrated that the use of such silk-based compositions for sustained release of an anti-vascular endothelial growth factor (VEGF) therapeutic agent (e.g., AVASTIN®, Genentech) to at least a portion of an eye for more than 3 months. More importantly, the inventors have surprisingly discovered that such silk based compositions can maintain an amount of an anti-VEGF therapeutic agent (e.g., AVASTIN®, Genentech) delivered to a target site (e.g., vitreous humor of an eye) at or above a therapeutically-effective level for at least about one month longer than when compared to the same amount of therapeutic agent being delivered by the current standard non-silk solution composition. Accordingly, an anti-VEGF therapeutic agent (e.g., AVASTIN®, Genentech) dispersed or encapsulated in a silk matrix can prolong a therapeutic effect in a subject over a period of time, which is at least one month longer than when compared to the same amount of the therapeutic agent being delivered in a current standard non-silk solution composition, thus significantly reducing the frequency of dosing for patients currently treated with such anti-VEGF therapeutic agents.

One aspect provided herein relates to compositions for ocular administration comprising a therapeutic agent dispersed or encapsulated in a silk matrix, wherein an amount of the therapeutic agent dispersed or encapsulated in the silk matrix can provide a therapeutic effect for a period of time which is longer than when the same amount of the therapeutic agent is administered without the silk matrix. In some embodiments, the therapeutic effect can be associated with treatment of an ocular condition, e.g., a reduction of at least one symptom associated with the ocular condition by at least about 10%.

In some embodiments, the amount of the therapeutic agent dispersed or encapsulated in the silk matrix can provide a therapeutic effect for a period of time which is at least about 1 week longer than when the same amount of the therapeutic agent is administered without the silk matrix.

In some embodiments, the amount of the therapeutic agent dispersed or encapsulated in the silk matrix can provide a therapeutic effect for a period of time which is at least about 1 month, at least about 2 months, at least about 3 months, or at least about 6 months longer than when the same amount of the therapeutic agent is administered without the silk matrix.

Generally, any therapeutic agents can be encapsulated or dispersed in a silk matrix. Exemplary types of therapeutic agents that can be encapsulated or dispersed in a silk matrix can include, but not limited to, proteins, peptides, antigens, immunogens, vaccines, antibodies or portions thereof, antibody-like molecules, enzymes, nucleic acids, siRNA, shRNA, aptamers, small molecules, antibiotics, and any combinations thereof.

In some embodiments, the therapeutic agent can be an agent for treatment of an ocular condition, e.g., without limitations, bevacizumab, ranibizumab, aflibercept, pegaptanib, tivozanib, fluocinolone acetonide, ganciclovir, triamcinolone acetonide, foscarnet, vancomycin, ceftazidime, amikacin, amphotericin B, dexamethasone, and any combinations thereof.

In one embodiment, the therapeutic agent, e.g., for treatment of an angiogenesis-induced condition such as in an eye, can be an angiogenesis inhibitor such as a VEGF inhibitor. Non-limiting examples of VEGF inhibitors can include bevacizumab, ranibizumab, aflibercept, pegaptanib, tivozanib, 3-(4-Bromo-2,6-difluoro-benzyloxy)-5-[3-(4-pyrrolidin 1-yl-butyl)-ureido]-isothiazole-4-carboxylic acid amide hydrochloride, axitinib, N-(4-bromo-2-fluorophenyl)-6-methoxy-7-[(1-methylpiperidin-4-yl)methoxy]quinazolin-4-amine, an inhibitor of VEGF-R2 and VEGF-R1, axitinib, N,2-dimethyl-6-(2-(1-methyl-1H-imidazol-2-yl)thieno[3,2-b]pyridin-7-yloxy)benzo[b]thiophene-3-carboxamide, tyrosine kinase inhibitor of the RET/PTC oncogenic kinase, N-(4-bromo-2-fluorophenyl)-6-methoxy-7-[(1-methylpiperidin-4-yl)methoxy]quinazolin-4-amine, pan-VEGF-R-kinase inhibitor; protein kinase inhibitor, multitargeted human epidermal receptor (HER) 1/2 and vascular endothelial growth factor receptor (VEGFR) 1/2 receptor family tyrosine kinases inhibitor, cediranib, sorafenib, vatalanib, glufanide disodium, VEGFR2-selective monoclonal antibody, angiozyme, an siRNA-based VEGFR1 inhibitor, Fumagillin and analogue thereof, soluble ectodomains of the VEGF receptors, shark cartilage and derivatives thereof, 5-((7-Benzyloxyquinazolin-4-yl)amino)-4-fluoro-2-methyl phenol hydrochloride, any derivatives thereof and any combinations thereof.

In one embodiment, the VEGF inhibitor, e.g., for treatment of an angiogenesis-induced condition such as in an eye can include bevacizumab, ranibizumab, or a combination thereof.

The amount of the therapeutic agent or the VEGF inhibitor dispersed or encapsulated in a silk matrix can range from nanograms to milligrams, depending on a number of factors, e.g., desirable release profile, properties and/or potency of the therapeutic agent or the VEGF inhibitor, severity of a condition to be treated, and administration schedule. In some embodiments, a therapeutic agent, e.g., a VEGF inhibitor, can be present in a silk matrix in an amount of from about 1 ng to about 100 mg, from about 0.01 mg to about 50 mg, or from about 5 mg to about 10 mg.

In some embodiments, the therapeutic agent, e.g., the VEGF inhibitor, can be present in an amount sufficient to maintain a therapeutically effective amount thereof delivered to at least a portion of an eye, upon administration, over a period of more than 1 month, more than 2 months, more than 3 months, more than 6 months or longer. Thus, in some embodiments, the composition is formulated for administration at least every month, at least every two months, at least every three months, at least every six months or longer. Such amounts of the therapeutic agent, e.g., the VEGF inhibitor, dispersed or encapsulated in a silk matrix can be generally smaller, e.g., at least about 10% smaller, than the amount of the therapeutic agent or the VEGF inhibitor dispersed or encapsulated in a non-silk matrix required for producing essentially the same therapeutic effect.

In one embodiment, the composition comprises bevacizumab, ranibizumab, or a combination thereof, encapsulated in a silk matrix, wherein about 0.5 mg to about 1.5 mg (e.g., about 1.25 mg) of bevacizumab, ranibizumab, or a combination thereof, encapsulated in the silk matrix provides a therapeutic effect for at least about 2 months, at least about 3 months or longer.

In one embodiment, the composition comprises bevacizumab, ranibizumab, or a combination thereof, encapsulated in a silk matrix, wherein about 1.5 mg to about 10 mg of bevacizumab, ranibizumab, or a combination thereof, encapsulated in the silk matrix can provide a therapeutic effect for at least about 3 months, at least about 4 months, at least about 5 months, at least about 6 months, at least about 9 months, at least about 12 months, at least about 18 months, at least about 24 months or longer. In some embodiments, about 3 mg to about 10 mg (e.g., about 5 mg) of bevacizumab, ranibizumab, or a combination thereof, encapsulated in the silk matrix can provide a therapeutic effect for at least about 3 months, at least about 4 months, at least about 5 months, at least about 6 months, at least about 9 months, at least about 12 months, at least about 18 months, at least about 24 months or longer.

Depending on the desired state or configuration of the silk matrix, e.g., hydrogel, microparticle, nanoparticle, fiber, film, lyophilized powder, lyophilized gel, reservoir implant, homogenous implant, gel-like or gel particle, and any combinations thereof, different concentrations of silk fibroin can be included in the silk matrix of the composition described herein. In some embodiments, a silk matrix can comprise silk fibroin at a concentration of about 0.1% (w/v) to about 50% (w/v), about 0.5% (w/v) to about 30% (w/v), or about 1% (w/v) to about 15% (w/v). In some embodiments, a silk matrix comprising silk fibroin can be produced from a silk solution containing silk fibroin at a concentration of about 0.1% (w/v) to about 30% (w/v), about 0.5% (w/v) to about 15% (w/v), or about 1% (w/v) to about 8% (w/v).

In one embodiment, the silk matrix, e.g., a hydrogel, comprising silk fibroin can be produced from a silk solution containing silk fibroin at a concentration of about 1% (w/v), about 2% (w/v), about 4% (w/v), about 6% (w/v), about 8% (w/v), about 10% (w/v), about 12% (w/v), about 15% (w/v), about 20% (w/v), about 25% (w/v), or about 30% (w/v) or higher silk fibroin. In some embodiments where higher concentration of silk fibroin are used, e.g., at least about 8% (w/v), at least about 10% (w/v), at least about 12% (w/v), at least about 15% (w/v), at least about 20% (w/v), at least about 25% (w/v), at least about 30% (w/v) or higher, the silk hydrogel can be reduced into gel-like or gel particles. The gel-like or gel particles can have a size ranging from 0.01 μm to about 1000 μm.

In one embodiment, the silk matrix, e.g., a microparticle or a nanoparticle, comprising silk fibroin can be produced from a silk solution containing silk fibroin at a concentration of about 1% (w/v), about 2% (w/v), about 4% (w/v), about 6% (w/v), about 8% (w/v), about 10% (w/v), about 12% (w/v), about 15% (w/v), about 20% (w/v), about 25% (w/v), or about 30% (w/v) or higher silk fibroin.

In one embodiment, the silk microparticle, nanoparticle or gel-like or gel particle, produced from a silk solution containing silk fibroin at a concentration of about 1% (w/v), about 2% (w/v), about 4% (w/v), about 6% (w/v), about 8% (w/v), about 10% (w/v), about 12% (w/v), about 15% (w/v), about 20% (w/v), about 25% (w/v), or about 30% (w/v) or higher silk fibroin, can be further embedded in a solid substrate and/or a biomaterial (e.g., a biocompatible material). Non-limiting examples of the solid substrate can include a tablet, a capsule, a microchip, a hydrogel, a mat, a film, a fiber, an ocular delivery device, an implant, a coating, and any combinations thereof. In some embodiments, the silk microparticle, nanoparticle or gel-like or gel particle can be further embedded in a solid matrix comprising silk fibroin, e.g., a silk matrix such as a silk hydrogel (with a silk concentration of about 0.25% (w/v) to about 2% (w/v) or about 0.5%(w/v) to about 1% (w/v), a biocompatible polymer, or a combination thereof. In some embodiments, the solid substrate and/or the biomaterial encapsulating the silk microparticle, nanoparticle, or gel-like or gel particle can be loaded with at least one therapeutic agent, which is same as or different from the therapeutic agent encapsulated in the silk microparticle, nanoparticle, or gel-like or gel particle.

In some embodiments, the silk matrix can further comprise a biocompatible polymer. The silk matrix can contain silk (e.g., comprising silk fibroin) blended with a biocompatible polymer, or silk conjugated to a biocompatible biopolymer. Exemplary biocompatible polymers include, but are not limited to, a poly-lactic acid (PLA), poly-glycolic acid (PGA), poly-lactide-co-glycolide (PLGA), polyesters, poly(ortho ester), poly(phosphazine), poly(phosphate ester), polycaprolactone, gelatin, collagen, cellulose, hyaluronan, poly(ethylene glycol) (PEG), triblock copolymers, polylysine and any derivatives thereof.

The compositions for ocular administration described herein can be formulated for various target sites of administration in an eye, e.g., but not limited to, lens, sclera, conjunctiva, aqueous humor, ciliary muscle, and vitreous humor. In some embodiments, the composition can be formulated to be an injectable composition, e.g., for intravitreal administration.

Different embodiments of the compositions described herein can be used to deliver at least one therapeutic agent to an eye and/or treat an ocular condition. Accordingly, another aspect provided herein relates to a method for delivering a therapeutic agent to an eye, which comprises administering to a target site of an eye a therapeutic agent dispersed or encapsulated in a silk matrix, wherein an amount of the therapeutic agent dispersed or encapsulated in the silk matrix can provide a therapeutic effect for a period of time which is longer than when the same amount of the therapeutic agent is administered without the silk matrix.

A further aspect described herein provides a method for treating an ocular condition in a subject, which comprises administering to a target site of an eye of a subject one or more embodiments of the composition described herein. In some embodiments, the composition can provide a sustained release of the therapeutic agent to the target site (e.g., including area in close proximity to the target site) of the eye, thereby treating the ocular condition in the subject.

An ocular condition can be any disease or disorder associated with any part of an eye. In some embodiments, the ocular condition can include a condition of a posterior segment of the eye. For example, the ocular condition can include, but not limited to, age-related macular degeneration, choroidal neovascularization, diabetic macular edema, acute and chronic macular neuroretinopathy, central serous chorioretinopathy, macular edema, acute multifocal placoid pigment epitheliopathy, Behcet's disease, birdshot retinochoroidopathy, posterior uveitis, posterior scleritis, serpignous choroiditis, subretinal fibrosis, uveitis syndrome, Vogt-Koyanagi-Harada syndrome, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, carotid artery disease (CAD), frosted branch angitis, sickle cell retinopathy, angioid streaks, familial exudative vitreoretinopathy, Eales disease, proliferative vitreal retinopathy, diabetic retinopathy, retinal disease associated with tumors, congenital hypertrophy of the retinal pigment epithelium (RPE), posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors, myopic retinal degeneration, acute retinal pigment epithelitis, glaucoma, endophthalmitis, cytomegalovirus retinitis, retinal cancers, and any combinations thereof.

In one embodiment, the ocular condition to be treated can be age-related macular degeneration. In such embodiments, the therapeutic agent dispersed or encapsulated in the silk matrix can include an angiogenesis inhibitor, e.g., a VEGF inhibitor. Exemplary VEGF inhibitors can comprise bevacizumab, ranibizumab, or a combination thereof.

Methods for increasing an effective amount of a therapeutic agent administered to a target site of an eye are also provided herein. In some embodiments, the method comprises administering to a target site of an eye a therapeutic agent dispersed or encapsulated in a silk matrix, wherein the silk matrix is formulated such that upon administration, leakage of the therapeutic agent from the target administration site is reduced, thereby increasing an effective amount of the therapeutic agent administered to the eye. In some embodiments, the leakage of the therapeutic agent from the target administration site can be reduced by at least about 5% or higher (including, e.g., at least about 10% or higher, at least about 20% or higher).

In various aspects described herein, the therapeutic agent dispersed or encapsulated in a silk matrix unexpectedly provides a therapeutic effect over a longer period of time than when the same amount of the therapeutic agent is dispersed or encapsulated without the silk matrix. Thus, when a subject is administered with one or more embodiments of the compositions described herein, the administration frequency of the composition can be reduced, when compared to a subject administered with the same amount of the therapeutic agent without the silk matrix. Accordingly, still another aspect provided herein relates to methods for administrating a therapeutic agent to a target site of an eye of a subject in need thereof, which comprises administrating to a target site of an eye of a subject one or more embodiments of the composition described herein at an administration frequency less than the administration frequency when the same amount of the therapeutic agent is administered without the silk matrix. In some embodiments, the administration frequency can be reduced by a factor of ½.

In some embodiments, the therapeutic effect produced by any aspects of the methods described herein can be associated with treatment of an ocular condition, e.g., a reduction of at least one symptom associated with an ocular condition by at least about 10%. In some embodiments, the therapeutic effect produced by any aspects of the methods described herein can sustain for a period of time, which is at least about 1 week longer than the duration of the therapeutic effect produced by the same amount of the therapeutic agent administered without the silk matrix. In some embodiments, the therapeutic effect produced by any aspects of the methods described herein can sustain for a period of time, which is at least about 1 month, at least about 2 months, at least about 3 months, or at least about 6 months, longer than the duration of the therapeutic effect produced by the same amount of the therapeutic agent administered without the silk matrix.

In any aspects of the methods described herein, the therapeutic agent dispersed or encapsulated in the silk matrix or the composition described herein can be administered to any parts of an eye, e.g., the anterior segment of the eye, or the posterior segment of the eye. In some embodiments, the therapeutic agent dispersed or encapsulated in the silk matrix or the composition described herein can be administered to at least a portion of the eye selected from the group consisting of lens, sclera, conjunctiva, aqueous humor, ciliary muscle, and vitreous humor. In one embodiment, the therapeutic agent dispersed or encapsulated in the silk matrix or the composition described herein can be administered to the vitreous humor of the eye.

In any aspects of the methods described herein, the therapeutic agent dispersed or encapsulated in the silk matrix or the composition described herein can be administered to the eye by any methods known in the art, e.g., injection or implantation. In some embodiments, the therapeutic agent dispersed or encapsulated in the silk matrix or the composition described herein can be administered to a target site of an eye by injection, e.g., intravitreal injection. The injection can performed with an injection needle suitable for eye injection, e.g., an injection needle with a gauge of about 25 to about 34, or about 27 to about 30.

In some embodiments of any aspects of the methods described herein, the administration of one or more embodiments of the compositions described herein, e.g., to a target site of an eye, can be performed no more than once a month, no more than once every two months, no more than once every three months, no more than once every four months, no more than once every five months, or no more once every six months or less frequently.

In any aspects of the methods described herein, the therapeutic agent dispersed or encapsulated in a silk matrix can be an agent of any type used for treatment of an ocular condition, e.g., without limitations, proteins, peptides, antigens, immunogens, vaccines, antibodies or portions thereof, antibody-like molecules, enzymes, nucleic acids, siRNA, shRNA, aptamers, small molecules, antibiotics, and any combinations thereof. Exemplary therapeutic agents can include, but not limited to, bevacizumab, ranibizumab, aflibercept, pegaptanib, tivozanib, fluocinolone acetonide, ganciclovir, triamcinolone acetonide, foscarnet, vancomycin, ceftazidime, amikacin, amphotericin B, dexamethasone, and any combinations thereof.

In some embodiments of any aspects of the methods described herein, the therapeutic agent can include an angiogenesis inhibitor, e.g., a VEGF inhibitor described herein. In certain embodiments, the VEGF inhibitor can include bevacizumab, ranibizumab, or a combination thereof. These particular embodiments of the methods described herein can be used for treatment of age-related macular degeneration.

In any aspects of the methods described herein, the amount of the therapeutic agent dispersed or encapsulated in a silk matrix can vary with desirable administration schedule, and/or release profiles of the therapeutic agent. For example, the therapeutic agent can be present in a silk matrix in an amount sufficient to maintain a therapeutically effective amount thereof delivered to at least a portion of an eye, upon administration, over a period of more than 1 month, including, e.g., more than 2 months, more than 3 months, more than 4 months, more than 5 months, more than 6 months or longer. In general, the longer the sustained release of the therapeutic agent to a target site, the less frequently the administration needs to be performed. In some embodiments, the therapeutic agent or the VEGF inhibitor can be present in a silk matrix in an amount of about 0.01 mg to about 50 mg, or about 5 mg to about 10 mg.

Depending on different administration methods, e.g., injection or implantation, and/or administration sites, different kinds of silk matrix can be used in different aspects of the methods described herein. For example, a silk matrix can be in a form of a hydrogel, microparticle, nanoparticle, fiber, film, lyophilized powder, lyophilized gel, reservoir implant, homogenous implant, gel-like or gel particle, or any combinations thereof. In some embodiments, the silk matrix can be a hydrogel, a microparticle or a nanoparticle, a gel-like or gel particle or any combinations thereof, which can be administered by a non-invasive method, e.g., injection.

In some embodiments, the silk microparticle, nanoparticle, or gel-like or gel particle can be further embedded in a biomaterial and/or solid substrate, e.g., but not limited to, a tablet, a capsule, a microchip, a hydrogel, a mat, a film, a fiber, an ocular delivery device, an implant, a coating, and any combinations thereof. In some embodiments, the silk microparticle, nanoparticle, or gel-like or gel particle can be further embedded in a solid substrate and/or a biomaterial, e.g., a silk matrix such as a silk hydrogel or a biocompatible polymer, e.g., to prolong a release profile of the therapeutic agent. In some embodiments, the silk microparticle, nanoparticle, or gel-like or gel particle can comprise at least one therapeutic agent at high concentrations/loading and can be further embedded into a solid substrate and/or biomaterial.

Different concentrations of silk fibroin can be used to achieve different kinds of silk matrices used in any embodiments described herein. In some embodiments, a silk matrix used in some embodiments of the methods described herein can comprise silk fibroin at a concentration of about 0.1% (w/v) to about 50% (w/v), about 0.5% (w/v) to about 30% (w/v), or about 1% (w/v) to about 15% (w/v). In some embodiments, a silk matrix used in some embodiments of the methods described herein can be produced from a silk solution containing silk fibroin at a concentration of about 0.1% (w/v) to about 30% (w/v), about 0.5% (w/v) to about 15% (w/v), or about 1% (w/v) to about 8% (w/v).

In one embodiment, the silk matrix, e.g., a hydrogel, used in the method can comprise silk fibroin produced from a silk solution containing silk fibroin at a concentration of about 1% (w/v), about 2% (w/v), about 4% (w/v), about 6% (w/v), about 8% (w/v), about 10% (w/v), about 12% (w/v), about 15% (w/v), about 20% (w/v), about 25% (w/v), or about 30% (w/v) or higher silk fibroin. In some embodiments where higher concentration of silk fibroin are used, e.g., at least about 8% (w/v), at least about 10% (w/v), at least about 12% (w/v), at least about 15% (w/v), at least about 20% (w/v), at least about 25% (w/v), at least about 30% (w/v) or higher, the silk hydrogel can be reduced into gel-like or gel particles. The gel-like or gel particles can have a size ranging from 0.01 μm to about 1000 μm.

In one embodiment, the silk matrix, e.g., a microparticle or a nanoparticle, used in the method can comprise silk fibroin produced from a silk solution containing silk fibroin at a concentration of about 4% (w/v), about 6% (w/v), about 8% (w/v), about 10% (w/v), about 12% (w/v), about 15% (w/v), about 20% (w/v), about 25% (w/v), or about 30% (w/v) or higher silk fibroin.

In one embodiment, the silk microparticle, nanoparticle, or gel-like or gel particle, produced from a silk solution containing silk fibroin at a concentration of about 4% (w/v), about 6% (w/v), about 8% (w/v), about 10% (w/v), about 12% (w/v), about 15% (w/v), about 20% (w/v), about 25% (w/v), or about 30% (w/v) or higher silk fibroin, can be further embedded in a biomaterial, e.g., a silk matrix such as a silk hydrogel (with a silk concentration of about 0.25% (w/v) to about 2% (w/v) or about 0.5%(w/v) to about 1% (w/v), a biocompatible polymer, or a combination thereof. In some embodiments, the solid substrate and/or the biomaterial, e.g., a silk matrix, encapsulating the silk microparticle, nanoparticle, or gel-like or gel particle can be loaded with a therapeutic agent, which is same as or different from the therapeutic agent encapsulated in the silk microparticle, nanoparticle, or gel-like or gel particle.

In some embodiments of any aspects of the methods described herein, the silk matrix can further comprise a biocompatible polymer described earlier. The silk matrix can contain silk (e.g., comprising silk fibroin) blended with a biocompatible polymer, or silk conjugated to a biocompatible biopolymer.

A therapeutic agent can be released, upon administration, from the silk matrix at any rate, which can be adjusted by varying, e.g., concentrations, and/or material state of the silk matrix. In some embodiments, the therapeutic agent can be released from the silk matrix at a rate such that at least about 20%, including, e.g., at least about 40% or at least about 60%, of the therapeutic agent initially encapsulated in the silk matrix can be released over a period of at least about 3 months or longer. In other embodiments, the therapeutic agent can be released from the silk matrix at the rate of about 1 ng/day to about 15 mg/day, or about 1 μg/day to about 1 mg/day.

Ocular delivery devices and kits, e.g., to facilitate administering any embodiments of the compositions and/methods described herein are also provided herein. In some embodiments, an ocular delivery device can comprise one or more embodiments of the composition described herein. An ocular delivery device can exist in any form, e.g., in some embodiments, the device can comprise a syringe with an injection needle, e.g., having a gauge of about 25 to about 34 or of about 27 to about 30. Other examples of an ocular delivery device that can be used for administration of the compositions and/or used in the methods described herein can include, but are not limited to, a contact lens, an eye-dropper, a microneedle (e.g., a silk microneedle), an implant, and any combinations thereof.

A kit provided herein can generally comprise at least one container containing one or more embodiments of the composition described herein, and/or at least one ocular delivery device in accordance with any embodiments described herein. In some embodiments, the composition can be pre-loaded into at least one ocular delivery device provided in the kit. For example, in one embodiment, the composition described herein can be pre-loaded into a syringe, which can be optionally attached with an injection needle. In some embodiments, e.g., where the composition is not provided or pre-loaded in a delivery device, the kit can further comprise, e.g., a syringe and an injection needle. In some embodiments, the kit can further comprise an anesthetic, e.g., an anesthetic that is commonly used during ocular administration. In some embodiments, the kit can further an antiseptic agent, e.g., to sterilize an administration site. In some embodiments, the kit can further comprise one or more swabs to apply the antiseptic agent onto the administration site.

### BRIEF DESCRIPTION OF THE DRAWINGS

FIG. 1 is a graph of rabbit body weight over the 90 day period following injection of negative vehicle control (i.e., silk hydrogel (˜2% silk) without therapeutic agent), positive control (i.e., ˜2.5% bevacizumab in solution), “low dose” silk hydrogel (i.e., ˜2% silk/˜2.5% bevacizumab, referred to therein as “low dose gel”) or “high dose” silk hydrogel (i.e., ˜2% silk/˜10% bevacizumab, referred to therein as “high dose gel”).

FIG. 2 illustrates bevacizumab concentration detected in vitreous humor collected from rabbits over a 90 day period following injection of negative vehicle control (i.e., silk hydrogel (˜2% silk) without therapeutic agent), positive control (i.e., ˜2.5% bevacizumab in solution), “low dose” silk hydrogel (i.e., ˜2% silk/˜2.5% bevacizumab, referred to therein as “low dose gel”) or “high dose” silk hydrogel (i.e., ˜2% silk/˜10% bevacizumab, referred to therein as “high dose gel”). Dotted lines represent the estimated Day 0 and Day 30 concentration of bevacizumab in rabbits subjected to the positive control treatment. The positive control treatment is used to mimic the current treatment being administered to a patient once a month. Based on the current dosage frequency of one injection per month, a representative therapeutic range of bevacizumab can be determined.

FIG. 3 illustrates bevacizumab concentration detected in aqueous humor collected from rabbits over a 90 day period following injection of negative vehicle control (i.e., silk hydrogel (˜2% silk) without therapeutic agent), positive control (i.e., ˜2.5% bevacizumab solution), “low dose” silk hydrogel (i.e., ˜2% silk/˜2.5% bevacizumab, referred to therein as “low dose gel”) or “high dose” silk hydrogel (i.e., ˜2% silk/˜10% bevacizumab, referred to therein as “high dose gel”). Dotted lines represent the estimated Day 0 and Day 30 concentration of bevacizumab in rabbits subjected to the positive control treatment. The positive control treatment is used to mimic the current treatment being administered to a patient once a month. Based on the current dosage frequency of one injection per month, a representative therapeutic range of bevacizumab can be determined.

FIG. 4 illustrates bevacizumab concentration detected in plasma collected from rabbits over a 90 day period following injection of negative vehicle control (i.e., silk hydrogel (˜2% silk) without therapeutic agent), positive control (i.e., ˜2.5% bevacizumab solution), “low dose” silk hydrogel (i.e., ˜2% silk/˜2.5% bevacizumab, referred to therein as “low dose gel”) or “high dose” silk hydrogel (i.e., ˜2% silk/˜10% bevacizumab, referred to therein as “high dose gel”).

FIGS. 5A-5B are representative terminal fundus photos taken at day 90 for rabbits injected with different formulations. FIG. 5A illustrates representative terminal fundus photos taken at day 90 for rabbits injected with negative vehicle control (i.e., silk hydrogel (˜2% silk) without therapeutic agent) or positive control (i.e., ˜2.5% bevacizumab solution). In particular, photos of control (left) eye and test (right) eye are provided with the inset for negative vehicle control being an image of the remaining hydrogel article. FIG. 5B illustrates representative terminal fundus photos taken at day 90 for rabbits injected with “low dose” hydrogel (i.e., ˜2% silk/˜2.5% bevacizumab, referred to therein as “low dose gel”) or “high dose” hydrogel (i.e., ˜2% silk/˜10% bevacizumab, referred to therein as “high dose gel”). In particular, photos of control (left) eye and test (right) eye are provided with the inset for “high dose” hydrogel being an image of the remaining hydrogel article. In some instances, the “low dose” hydrogel article is obstructing the optic disc. FIGS. 5A-5B show reduced growth of retinal blood vessels at day 90 post-treatment in the rabbits treated with silk hydrogels loaded with bevacizumab, as compared to the negative and positive controls.

FIG. 6 is a graph which illustrates degradation of different formulations as visually scored during ophthalmic examinations of rabbits over a 90 day period following injection of negative vehicle control (i.e., silk hydrogel (˜2% silk) without therapeutic agent), positive control (i.e., ˜2.5% bevacizumab solution), “low dose” silk hydrogel (i.e., ˜2% silk/˜2.5% bevacizumab, referred to therein as “low dose gel”) or “high dose” silk hydrogel (i.e., ˜2% silk/˜10% bevacizumab, referred to therein as “high dose gel”).

FIG. 7 is a graph which illustrates bevacizumab concentration in vitro over a 90 day period following injection in PBS (with ˜0.02% sodium azide) of negative vehicle control (i.e., silk hydrogel (˜2% silk) without therapeutic agent), positive control (i.e., ˜2.5% bevacizumab solution), “low dose” silk hydrogel (i.e., ˜2% silk/˜2.5% bevacizumab, referred to therein as “low dose gel”) or “high dose” silk hydrogel (i.e., ˜2% silk/˜10% bevacizumab, referred to therein as “high dose gel”).

### DETAILED DESCRIPTION OF THE INVENTIONS

There is a need for improved pharmaceutical compositions for ocular administrations and/or therapeutic interventions that can provide sustained delivery of therapeutic agent(s), e.g., anti-angiogenic agent(s), with greater patient comfort and thus greater patient compliance. Embodiments of various aspects described herein relate to compositions, ocular delivery devices, kits and methods for sustained delivery of a therapeutic agent to at least a portion of an eye and/or for treatment of an ocular condition, e.g., angiogenesis-induced ocular disease or disorder such as age-related macular degeneration. The compositions described herein generally comprise a therapeutic agent, e.g., an angiogenesis inhibitor, including a VEGF inhibitor, dispersed or encapsulated in a silk matrix, e.g., but not limited to, a hydrogel.

The inventors have demonstrated that, in some embodiments, a therapeutic agent, e.g., a VEGF inhibitor such as AVASTIN® dispersed or encapsulated in a silk matrix, e.g., a silk hydrogel, can be formulated for sustained release of at least about 3 months or longer. The new silk-based composition described herein can, in some embodiments, allow safe administration of an amount of a therapeutic agent, e.g., a VEGF inhibitor, which is at least about 30% or more (including as high as about 4-fold), higher than the amount of the same therapeutic agent allowed for administration in one dose using the current non-silk administration. More surprisingly, in some embodiments, even when the silk-based composition contains the same amount of the therapeutic agent (e.g., a VEGF inhibitor such as AVASTIN®) as the amount contained in one dosage of the current non-silk composition, the silk-based composition can provide a sustained release of the therapeutic agent at a level of at least about or above a therapeutically-effective amount, for a longer period of time, e.g., at least about 1 week longer or even about at least about 1 month longer, as compared to administration with the current non-silk composition. In some embodiments, the silk-based composition can provide a therapeutic effect for a longer period of time, e.g., at least about 1 week longer or even about at least about 1 month longer, as compared to administration with the current non-silk composition. Accordingly, some embodiments of the compositions described herein can be used to reduce the frequency of dosing for patients currently treated with an anti-VEGF agent. Further, in some embodiments, the silk matrix encapsulating the therapeutic agent, upon administration, can degrade in vivo into biocompatible amino acids over time, e.g., after about 3 months or longer. Thus, the silk-based compositions can be administrated repeatedly, if needed, without concerns about extracting the previously-administered silk matrix before a new administration.

In one aspect, described herein relates to compositions for ocular administration comprising a therapeutic agent dispersed or encapsulated in a silk matrix, wherein an amount of the therapeutic agent dispersed or encapsulated in the silk matrix can provide a therapeutic effect for a period of time which is longer than when essentially the same amount of the therapeutic agent is administered without the silk matrix.

In some embodiments, the therapeutic effect can be associated with treatment of an ocular condition, e.g., an angiogenesis-induced ocular condition such as age-related macular degeneration. In some embodiments, the therapeutic effect can be determined by detecting a reduction of at least one symptom associated with the ocular condition by at least about 10% or higher, as compared to a control reference, e.g., when the therapeutic agent is not administered, or when the same therapeutic agent is administered without the silk matrix. The term “therapeutic effect” as used herein is discussed further in details below.

In some embodiments, the amount of the therapeutic agent dispersed or encapsulated in the silk matrix can provide a therapeutic effect for any period of time longer than when the same amount of the therapeutic agent is administered without the silk matrix. In some embodiments, the period of time can range from days to weeks to months. For example, in some embodiments, the amount of the therapeutic agent dispersed or encapsulated in the silk matrix can provide a therapeutic effect for a period of time, which is at least about 1 day, at least about 2 days, at least about 3 days, at least about 4 days, at least about 5 days, at least about 6 days, or at least about 7 days or more, longer than when the same amount of the therapeutic agent is administered without the silk matrix. In other embodiments, the amount of the therapeutic agent dispersed or encapsulated in the silk matrix can provide a therapeutic effect for a period of time, which is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months or more, longer than when the same amount of the therapeutic agent is administered without the silk matrix.

In some embodiments, the amount of the therapeutic agent dispersed or encapsulated in the silk matrix can provide a therapeutic effect for at least about 1 month longer than when the same amount of the therapeutic agent is administered without the silk matrix. In some embodiments, the amount of the therapeutic agent dispersed or encapsulated in the silk matrix can provide a therapeutic effect for at least about 3 months longer than when the same amount of the therapeutic agent is administered without the silk matrix. In some embodiments, the amount of the therapeutic agent dispersed or encapsulated in the silk matrix can provide a therapeutic effect for at least about 6 months longer than when the same amount of the therapeutic agent is administered without the silk matrix. In some embodiments, the amount of the therapeutic agent dispersed or encapsulated in the silk matrix can provide a therapeutic effect for at least about 12 months longer than when the same amount of the therapeutic agent is administered without the silk matrix.

As used herein, the phrase “the therapeutic agent is administered without the silk matrix” generally refers to the therapeutic agent dispersed or encapsulated in a non-silk matrix, or the therapeutic agent administered in combination with a non-silk matrix. The therapeutic agent without the silk matrix can be administered using the same method as or a different method from the one used for administering the composition described herein. The therapeutic agent administered without the silk matrix can be formulated to a format or state same as or different from the format or state formulated for the composition described herein. In some embodiments, the non-silk matrix can be in the form of a solution comprising essentially no silk or no silk fibroin, e.g., a buffered solution. In some embodiments, the non-silk matrix can be in the form of a gel, a microparticle, a nanoparticle, a fiber, a film, or an implant, comprising essentially no silk or no silk fibroin. In some embodiments, the non-silk matrix can comprise a biocompatible non-silk polymer, e.g., poly-lactide-co-glycolide (PLGA).

In some embodiments, the amount of the therapeutic agent dispersed or encapsulated in the silk matrix can provide a therapeutic effect for at least about 1 week, including, e.g., at least about 1 month, at least about 3 months, at least about 6 months, at least about 12 months or more, longer than when essentially the same amount of the therapeutic agent is administered in a non-silk buffered solution.

### Amounts of a Therapeutic Agent in a Silk Matrix

Generally, any amount of a therapeutic agent can be dispersed or encapsulated in a silk matrix, depending on a number of factors, including, but not limited to, desirable release profile (e.g., release rates and/or duration), properties (e.g., half-life and/or molecular size) and/or potency of the therapeutic agent, severity of a subject's ocular condition to be treated, desirable administration schedule, loading capacity of the silk matrix, physical condition of the subject to be treated, and any combinations thereof. For example, in some embodiments, a therapeutic agent can be present in a silk matrix in an amount of about 1 ng to about 100 mg, about 500 ng to about 90 mg, about 1 μg to about 75 mg, about 0.01 mg to about 50 mg, about 0.1 mg to about 50 mg, about 1 mg to about 40 mg, about 5 mg to about 25 mg. In some embodiments, a therapeutic agent can be present in a silk matrix in an amount of about 0.01% (w/v) to about 90% (w/v) of the total silk matrix volume (i.e., the combined volume of the silk matrix and the therapeutic agent), for example, including, about 0.05% (w/v) to about 75% (w/v), about 0.1% (w/v) to about 50% (w/v), about 1% (w/v) to about 40% (w/v), about 5% (w/v) to about 25% (w/v), or about 7.5% (w/v) to about 20 (w/v) of the total silk matrix volume. In some embodiments, the therapeutic agent can be present in a silk matrix in an amount of about 0.5% (w/v) to about 50% (w/v) of the total silk matrix volume. In some embodiments, the therapeutic agent can be present in a silk matrix in an amount of about 3% (w/v) to about 50% (w/v) of the total silk matrix volume. In one embodiment, the therapeutic agent (e.g., an anti-VEGF inhibitor such as AVASTIN® or LUCENTIS®) can be present in a silk matrix in an amount of about 0.1% (w/v) to about 20% (w/v), or about 0.5% (w/v) to about 10% (w/v), or about 0.5% (w/v) to about 3% (w/v), of the total silk matrix volume. In one embodiment, the therapeutic agent (e.g., an anti-VEGF inhibitor such as AVASTIN® or LUCENTIS®) can be present in a silk matrix in an amount of about 3% (w/v) to 20% (w/v) of the total silk matrix volume. In one embodiment, the therapeutic agent (e.g., an anti-VEGF inhibitor such as AVASTIN® or LUCENTIS®) can be present in a silk matrix in an amount of about 5% (w/v) to 10% (w/v) of the total silk matrix volume.

Without wishing to be bound by theory, the duration of a therapeutic effect on a target site (e.g., in an eye) to be treated is generally correlated with how long an amount of a therapeutic agent delivered to the target site can be maintained at a therapeutically effective amount. Thus, in some embodiments, compositions for ocular administration can comprise a therapeutic agent dispersed or encapsulated in a silk matrix, wherein the therapeutic agent is present in an amount sufficient to maintain a release of the therapeutic agent from the silk matrix to a target site of an eye or close proximity thereof, upon administration, at a therapeutically effective amount over a specified period of time, e.g., over more than 1 month.

The term “therapeutically effective amount” as used herein refers to an amount of a therapeutic agent which is effective for producing a beneficial or desired clinical result in at least a sub-population of cells in a subject at a reasonable benefit/risk ratio applicable to any medical treatment. For example, a therapeutically effective amount delivered to a target site or close proximity thereof, e.g., at least a portion of an eye (e.g., vitreous humor) and/or ocular cells (e.g., retinal cells) is sufficient to, directly or indirectly, produce a statistically significant, measurable therapeutic effect as defined herein. By way of example only, when an ocular condition to be treated is an angiogenesis-induced ocular disease or disorder (e.g., age-related macular degeneration), the therapeutically effective amount delivered to at least a portion of an eye (e.g., vitreous humor) and/or ocular cells (e.g., retinal cells) is sufficient to reduce at least one symptom or marker associated with the angiogenesis-induced ocular disease or disorder (e.g., age-related macular degeneration) by at least about 10%, at least about 20%, at least about 30%, at least about 40%, at least about 50%, at least about 60% or higher (but excluding 100%), as compared to absence of the therapeutic agent. In some embodiments, the therapeutically effective amount delivered to at least a portion of an eye (e.g., vitreous humor) and/or ocular cells (e.g., retinal cells) is sufficient to reduce at least one symptom or marker associated with the angiogenesis-induced ocular disease or disorder (e.g., age-related macular degeneration) by at least about 60%, at least about 70%, at least about 80% or higher (but excluding 100%), as compared to absence of the therapeutic agent. In some embodiments, the therapeutically effective amount delivered to at least a portion of an eye (e.g., vitreous humor) and/or ocular cells (e.g., retinal cells) is sufficient to reduce at least one symptom or marker associated with the angiogenesis-induced ocular disease or disorder (e.g., age-related macular degeneration) by at least about 80%, at least about 90%, at least about 95%, at least about 98%, at least about 99% or higher (but excluding 100%), as compared to absence of the therapeutic agent. In some embodiments, the therapeutically effective amount delivered to at least a portion of an eye (e.g., vitreous humor) and/or ocular cells (e.g., retinal cells) is sufficient to reduce at least one symptom or marker associated with the angiogenesis-induced ocular disease or disorder (e.g., age-related macular degeneration) by 100%, as compared to absence of the therapeutic agent.

Exemplary symptoms of an angiogenesis-induced ocular disease or disorder (e.g., age-related macular degeneration) that can be treated with one or more embodiments of the compositions and/or methods described herein can include, but are not limited to, proliferation of abnormal blood vessels in the retina of an eye, and reduced vision. In some embodiments where age-related macular degeneration (AMD) is to be treated, the therapeutically effective amount delivered to the vitreous humor of an eye diagnosed with AMD and/or its ocular cells (e.g., retinal cells) is sufficient to induce regression and/or inhibit proliferation of abnormal blood vessels in the retina by at least about 10%, at least about 20%, at least about 30%, at least about 40%, at least about 50%, at least about 60% or higher (but excluding 100%), as compared to absence of the therapeutic agent. In some embodiments, the therapeutically effective amount delivered to the vitreous humor of an eye diagnosed with AMD and/or its ocular cells (e.g., retinal cells) is sufficient to induce regression and/or inhibit proliferation of abnormal blood vessels in the retina by at least about 60%, at least about 70%, at least about 80% or higher (but excluding 100%), as compared to absence of the therapeutic agent. In some embodiments, the therapeutically effective amount delivered to the vitreous humor of an eye diagnosed with AMD and/or its ocular cells (e.g., retinal cells) is sufficient to induce regression and/or inhibit proliferation of abnormal blood vessels in the retina by at least about 80%, at least about 90%, at least about 95%, at least about 98%, at least about 99% or higher (but excluding 100%), as compared to absence of the therapeutic agent. In one embodiment, the therapeutically effective amount delivered to the vitreous humor of an eye diagnosed with AMD and/or ocular cells (e.g., retinal cells) is sufficient to induce regression and/or inhibit proliferation of abnormal blood vessels in the retina by 100%, as compared to absence of the therapeutic agent.

In some embodiments where age-related macular degeneration (AMD) is to be treated, the therapeutically effective amount delivered to the vitreous humor of an eye diagnosed with AMD and/or its ocular cells (e.g., retinal cells) is sufficient to improve vision (e.g., but not limited to, reduced blurring in central vision, reduced visual distortion and/or hallucinations) by at least about 10%, at least about 20%, at least about 30%, at least about 40%, at least about 50%, at least about 60% or higher (but excluding 100%), as compared to absence of the therapeutic agent. In some embodiments, the therapeutically effective amount delivered to the vitreous humor of an eye diagnosed with AMD and/or its ocular cells (e.g., retinal cells) is sufficient to improve vision (e.g., but not limited to, reduced blurring in central vision, reduced visual distortion and/or hallucinations) by at least about 60%, at least about 70%, at least about 80% or higher (but excluding 100%), as compared to absence of the therapeutic agent. In some embodiments, the therapeutically effective amount delivered to the vitreous humor of an eye diagnosed with AMD and/or its ocular cells (e.g., retinal cells) is sufficient to improve vision (e.g., but not limited to, reduced blurring in central vision, reduced visual distortion and/or hallucinations) by at least about 80%, at least about 90%, at least about 95%, at least about 98%, at least about 99% or higher (but excluding 100%), as compared to absence of the therapeutic agent. In one embodiment, the therapeutically effective amount delivered to the vitreous humor of an eye diagnosed with AMD and/or ocular cells (e.g., retinal cells) is sufficient to improve vision (e.g., but not limited to, reduced blurring in central vision, reduced visual distortion and/or hallucinations) by 100%, as compared to absence of the therapeutic agent.

Determination of a therapeutically effective amount is well within the capability of those skilled in the art. Generally, a therapeutically effective amount can vary with, for example, the subject's history, age, condition, sex, as well as the severity and type of the medical condition in the subject, and/or administration of other pharmaceutically active agents. Furthermore, the therapeutically effective amounts can vary, as recognized by those skilled in the art, depending on the specific disease treated, the route of administration, the excipient selected, and the possibility of combination therapy, e.g., laser coagulation and/or surgery. In some embodiments, the therapeutically effective amount can be in a range between the ED50 and LD50 (a dose of a therapeutic agent at which about 50% of subjects taking it are killed). In some embodiments, the therapeutically effective amount can be in a range between the ED50 (a dose of a therapeutic agent at which a therapeutic effect is detected in at least about 50% of subjects taking it) and the TD50 (a dose at which toxicity occurs at about 50% of the cases). In alternative embodiments, the therapeutically effective amount can be an amount determined based on the current dosage regimen of the same therapeutic agent administered in a non-silk matrix. For example, an upper limit of the therapeutically effective amount can be determined by a concentration or an amount of the therapeutic agent delivered to at least a portion of an eye, e.g., vitreous humor, on the day of administration with the current dosage of the therapeutic agent in a non-silk matrix; while the lower limit of the therapeutically effective amount can be determined by a concentration or an amount of the therapeutic agent delivered to at least a portion of an eye, e.g., vitreous humor, on the day at which a fresh dosage of the therapeutic agent in a non-silk matrix is required.

As used herein, the term “maintain” is used in reference to sustaining a concentration or an amount of a therapeutic agent delivered to a target site of an eye at least about or above the therapeutically effective amount over a specified period of time. In some embodiments, the term “maintain” as used herein can refer to keeping the concentration or amount of a therapeutic agent at an essentially constant value over a specified period of time. In some embodiments, the term “maintain” as used herein can refer to keeping the concentration or amount of a therapeutic agent within a range over a specified period of time. For example, the concentration or amount of a therapeutic agent delivered to a target site of an eye can be maintained within a range between about the ED50 and about the LD50 or between about the ED50 and about the TD50 over a specified period of time. In such embodiments, the concentration or amount of a therapeutic agent delivered to a target site of an eye can vary with time, but is kept within the therapeutically effective amount range for at least 90% of the specified period of time (e.g., at least about 95%, about 98%, about 99%, up to and including 100%, of the specified period of time).

In some embodiments, the therapeutic agent is present in an amount sufficient to maintain a release or delivery of the therapeutic agent from the silk matrix to a target site of an eye or close proximity thereof, upon administration, at a therapeutically effective amount over a specified period of time, over a period of more than 1 month, including, e.g., at least about 2 months, at least about 3 months, at least about 6 months, at least about 12 months or longer. Such amounts of the therapeutic agent dispersed or encapsulated in a silk matrix can be generally smaller, e.g., at least about 10% smaller, than the amount of the therapeutic agent present in the current dosage of the treatment regimen (i.e., without silk matrix) required for producing essentially the same therapeutic effect. Indeed, the inventors have discovered that a therapeutic agent encapsulated in a silk matrix can increase duration of the therapeutic effect for the therapeutic agent. Stated another way, the inventors have discovered that encapsulating a therapeutic agent in a silk matrix can increase its therapeutic efficacy, i.e., a smaller amount of a therapeutic agent encapsulated in a silk matrix, as compared to the amount present in a typical one dosage administered for a particular indication, e.g., an ocular condition such as angiogenesis-induced ocular condition (e.g., age-related macular degeneration), can achieve essentially the same therapeutic effect. Accordingly, the silk matrix can comprise the therapeutic agent in an amount which is less than the amount traditionally recommended for one dosage of the therapeutic agent, while achieving essentially the same therapeutic effect. For example, if the traditionally recommended dosage of the therapeutic agent is X amount then the silk matrix can comprise a therapeutic agent in an amount of about 0.9X, about 0.8X, about 0.7X, about 0.6X, about 0.5X, about 0.4X, about 0.3X, about 0.2X, about 0.1X or less. Without wishing to be bound by the theory, this can allow administering a lower dosage of the therapeutic agent in a silk matrix to obtain a therapeutic effect which is similar to when a higher dosage is administered without the silk matrix. Low-dosage administration of the therapeutic agent can reduce side effects of the therapeutic agent, if any, and/or reduce likelihood of the subject's resistance to the therapeutic agent after administration for a period of time.

In some embodiments, an amount of the therapeutic agent dispersed or encapsulated in the silk matrix can be more than the amount generally recommended for one dosage of the same therapeutic agent administered for a particular indication, e.g., an ocular condition such as angiogenesis-induced ocular condition (e.g., age-related macular degeneration). By way of example only, about 0.5 mg to about 1.25 mg of bevacizumab has been generally intravitreally administered as a solution to an eye of a subject, e.g., for treatment of age-related macular degeneration. Administration of a therapeutic agent (e.g., bevacizumab) in solution does not generally allow controlled and sustained release. Thus, release rate of a therapeutic agent in solution can generally create a higher initial burst and/or overall faster release kinetics than that of the same amount of the therapeutic agent loaded in silk matrix. Because of such higher initial burst observed in solution delivery, a current single dosage administered to an eye of a subject generally contains a limited amount of bevacizumab in solution, e.g., to ensure that the initial burst concentration does not go beyond the toxic level. Therefore, the current dosage of the treatment regimen (e.g., for treatment of age-related macular degeneration) requires administration of bevacizumab in solution at least once every month, in order to maintain the therapeutic effect. To this end, the inventors have demonstrated, in some embodiments, that not only can encapsulating bevacizumab in silk matrix prolong a therapeutic effect by at least 1 month, but they have also shown that the silk matrix can act as a depot such that the total amount of the therapeutic agent loaded in a silk matrix can be higher than the amount generally recommended for one dosage of the same therapeutic agent (e.g., 5 mg bevacizumab in a silk matrix as compared to 1.25 mg bevacizumab in current solution dosage), thus providing a longer therapeutic effect with lower frequency of administration. Accordingly, if the recommended dosage of the therapeutic agent is X amount then the silk matrix can encapsulate a therapeutic agent in an amount of about 1.25X, about 1.5X, about 1.75X, about 2X, about 2.5X, about 3X, about 4X, about 5X, about 6X, about 7X, about 8X, about 9X, about 10X or more. Without wishing to be bound by a theory, in these embodiments, the therapeutic agent encapsulated in the silk matrix administered to a target site of an eye can provide a similar therapeutic effect obtained with multiple administrations of the therapeutic agent without the silk matrix.

In some embodiments where the therapeutic agent is loaded in a silk matrix at a high concentration, the therapeutic agent can be first encapsulated into silk microparticles, silk nanoparticles, gel-like or gel particles, or any combinations thereof, which are then further embedded in a solid substrate and/or biomaterial described herein. In one embodiment, the silk microparticles, silk nanoparticles, gel-like and/or gel particles encapsulating the therapeutic agent can be further embedded in a hydrogel, e.g., comprising a silk hydrogel.

In some embodiments, an amount of the therapeutic agent encapsulated in the silk matrix can be essentially the same amount generally recommended for one dosage of the therapeutic agent, but providing a longer therapeutic effect. For example, if the generally recommended dosage of the therapeutic agent is X amount, then the silk-based composition can comprise about X amount of the therapeutic agent. Without wishing to be bound by the theory, in these embodiments, the therapeutic agent encapsulated in the silk matrix can permit fewer administrations of the therapeutic agent than the existing treatment regime, as the silk-based composition can provide a therapeutic effect over a longer period of time than the therapeutic agent without the silk matrix.

As used herein, the term “sustained delivery” refers to continual delivery of a therapeutic agent in vivo or in vitro over a period of time following administration. For example, sustained release can occur over a period of at least about 3 days, at least about a week, at least about two weeks, at least about three weeks, at least about four weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months or longer. In some embodiments, the sustained release can occur over a period of more than one month or longer. In some embodiments, the sustained release can occur over a period of at least about three months or longer. In some embodiments, the sustained release can occur over a period of at least about six months or longer. In some embodiments, the sustained release can occur over a period of at least about nine months or longer. In some embodiments, the sustained release can occur over a period of at least about twelve months or longer.

Sustained delivery of the therapeutic agent in vivo can be demonstrated by, for example, the continued therapeutic effect (e.g., reducing at least one symptom associated with an ocular condition, e.g., age-related macular degeneration) of the therapeutic agent over time. Alternatively, sustained delivery of the therapeutic agent can be demonstrated by detecting the presence or level of the therapeutic agent in vivo over time. By way of example only, sustained delivery of the therapeutic agent, upon intravitreal administration, can be detected by measuring the amount of therapeutic agent present in aqueous humor, vitreous humor and/or blood serum of a subject. The release rate and/or release profile of a therapeutic agent can be adjusted by a number of factors such as silk matrix composition and/or concentration, porous property (e.g., pore size and/or porosity) of the silk matrix, amounts and/or molecular size of the therapeutic agent loaded in a silk matrix, contents of beta-sheet structures in a silk matrix, and/or interaction of the therapeutic agent with the silk matrix (e.g., binding affinity of the therapeutic agent to a silk matrix), and any combinations thereof. For example, if the therapeutic agent has a higher affinity with the silk matrix, the release rate is usually slower than the one with a lower affinity with the silk matrix. Additionally, when a silk matrix has larger pores, the encapsulated therapeutic agent is generally released from the silk matrix faster than from a silk matrix with smaller pores.

In some embodiments, the therapeutic agent can be first encapsulated into silk microparticles, silk nanoparticles, gel-like or gel particles, or any combinations thereof, which are then further embedded in a solid substrate and/or biomaterial described herein, e.g., in order to control release of the therapeutic agent to a target site of an eye. In one embodiment, the silk microparticles, silk nanoparticles, gel-like and/or gel particles encapsulating the therapeutic agent can be further embedded in a hydrogel, e.g., comprising a silk hydrogel.

In some embodiments, high concentrations/loading of the therapeutic agent can be encapsulated in a silk matrix, e.g., to promote a sustained release. For example, in some embodiments, high concentrations/loading of the therapeutic agent can be first encapsulated into silk microparticles, silk nanoparticles, gel-like or gel particles, or any combinations thereof, which are then further embedded in a solid substrate and/or biomaterial described herein. In one embodiment, the silk microparticles, silk nanoparticles, gel-like and/or gel particles encapsulating high concentrations/loading of the therapeutic agent can be further embedded in a hydrogel, e.g., comprising a silk hydrogel.

In some embodiments, the therapeutic agent can be loaded in a silk matrix in an amount sufficient to provide a sustained delivery of the therapeutic agent, upon administration, to a target site of an eye (e.g., vitreous humor) within a therapeutically effective amount range. In some embodiments, the therapeutic agent can be loaded in a silk matrix in an amount sufficient to maintain the release rate of the therapeutic agent at about 0.01 ng/day to about 1000 mg/day, at about 0.1 ng/day to about 500 mg/day, or at about 1 ng/day to about 250 mg/day, over a period of time.

In some embodiments, upon administration of a therapeutic agent encapsulated or dispersed in a silk matrix or a composition described herein, there can be an initial spike in the amount of the therapeutic agent delivered to a target site, and then the release rate of the therapeutic agent from the silk matrix can be decreasing over a period of time. Thus, the therapeutic agent can be released initially at a rate as high as mg/day, and later released at a slower rate, e.g., in μg/day or ng/day. Accordingly, in some embodiments, the therapeutic agent can be loaded in a silk matrix in an amount that can yield an initial release rate of about 0.01 mg/day to about 1000 mg/day, about 0.1 mg/day to about 500 mg/day, or from about 1 mg/day to about 250 mg/day, upon administration, e.g., at least about 1 day after administration, including, e.g., at least about 2 days, at least about 3 days, at least about 4 days, at least about 5 days, at least about 6 days, at least about 7 days, at least about two weeks or longer, after administration. In some embodiments, the therapeutic agent can be loaded in a silk matrix in an amount that can yield a release rate of about 0.01 ng/day to about 10 μg/day, about 0.1 ng/day to about 1 μg/day, about 1 ng/day to about 500 ng/day, about 5 ng/day to about 250 ng/day, or about 10 ng/day to about 200 ng/day, upon administration, e.g., at least about 1 week after administration, including, e.g., at least about 2 weeks, at least about 3 weeks, at least about 4 weeks, at least about 2 months, at least about 3 months, at least about 6 months, at least 9 months, at least about 12 months or longer, after administration. In some embodiments, the therapeutic agent during such period of time can be released even at a lower rate, e.g., in pg/day level.

Stated another way, the therapeutic agent can be released from the silk matrix at a rate such that at least about 20%, including, e.g., at least about 30%, at least about 40%, at least about 50%, at least about 60%, at least about 70%, at least about 80%, at least about 90% or more, of the therapeutic agent initially encapsulated in the silk matrix can be released over a period of about 1 month, about 2 months, about 3 months, about 4 months, about 5 months, about 6 months, about 7 months, about 8 months, about 9 months, about 10 months, about 11 months, about 12 months or longer.

At least one therapeutic agent can be dispersed or encapsulated in the silk matrix. In some embodiments, at least two or more therapeutic agents can be dispersed or encapsulated in the silk matrix. The therapeutic agent can be present in any form suitable for a particular method to be used for encapsulation and/or dispersion. For example, the therapeutic agent can be in the form of a solid, liquid, or gel. In some embodiments, the therapeutic agent can be in the form of a powder or a pellet. In some embodiments, the therapeutic agent can be dispersed or encapsulated in a silk solution or matrix before forming the silk matrix. In some embodiments, the therapeutic agent can be dispersed or encapsulated in a silk solution or matrix after forming the silk matrix. For example, the therapeutic agent can be dispersed homogeneously or heterogeneously within the silk matrix, e.g., by pre-loading or post-loading silk fibroin solution, e.g., as described in the U.S. Provisional Application No. 61/545,786, the International Application No. WO/2011/109691, and U.S. Pat. No. 8,178,656, or dispersed in a gradient, e.g., using the carbodiimide-mediated modification method described in the U.S. Patent Application No. US 2007/0212730. In some embodiments, the therapeutic agent can be coated on a surface of the silk matrix, e.g., via diazonium coupling reaction (see, e.g., U.S. Patent Application No. US 2009/0232963), and/or avidin-biotin interaction (see, e.g., International Application No.: WO 2011/011347). In some embodiments, the therapeutic agent can be encapsulated in the silk matrix, e.g., by blending the therapeutic agent into a silk solution before processing into a desired material state, e.g., a hydrogel, or a microsphere or a nanosphere. See, e.g., U.S. Pat. No. 8,187,616; and U.S. Pat. App. Nos. US 2008/0085272, US 2010/0028451, US 2012/0052124, US 2012/0070427, and US 2012/0187591, the contents of which are incorporated herein by reference. In some embodiments, the therapeutic agent can be present in a form of a fusion protein with silk protein, e.g., by genetically engineering silk to generate a fusion protein comprising the therapeutic agent.

In some embodiments, the therapeutic agent can be dispersed or encapsulated in a silk matrix after the silk matrix is formed, e.g., by placing the formed silk matrix in a therapeutic agent solution and allowing the therapeutic agent to diffuse into the silk matrix over a period of time, e.g., by post-loading silk fibroin solution, e.g., as described in the U.S. Provisional Application No. 61/545,786, and the International Application No. WO/2011/109691, the contents of which are incorporated herein by reference. In some embodiments, the silk matrix can be optionally hydrated before loading with the therapeutic agent. For example, the silk matrix can be incubated in deionized water until completely hydrated.

### Silk Matrix or Silk-Based Composition

As used herein, the phrases “silk matrix” or “silk-based composition” generally refer to a matrix or a composition comprising silk. In some embodiments, silk can exclude sericin. In some embodiments, silk can comprise silk fibroin, silk sericin or a combination thereof. The phrases “silk matrix” and “silk-based composition” refer to a matrix or composition in which silk (or silk fibroin) constitutes at least about 30% of the total silk matrix composition, including at least about 40%, at least about 50%, at least about 60%, at least about 70%, at least about 80%, at least about 90%, at least about 95%, up to and including 100% or any percentages between about 30% and about 100%, of the total silk matrix composition. In certain embodiments, the silk matrix can be substantially formed from silk or silk fibroin. In various embodiments, the silk matrix can be substantially formed from silk or silk fibroin comprising at least one therapeutic agent.

As used herein, the term “silk fibroin” includes silkworm fibroin and insect or spider silk protein. See e.g., Lucas et al., 13 Adv. Protein Chem. 107 (1958). Any type of silk fibroin can be used in different embodiments of various aspects described herein. Silk fibroin produced by silkworms, such as Bombyx mori, is the most common and represents an earth-friendly, renewable resource. For instance, silk fibroin used in a silk fibroin fiber can be attained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants (see, e.g., WO 97/08315; U.S. Pat. No. 5,245,012), and variants thereof, that can be used. In some embodiments, silk fibroin can be derived from other sources such as spiders, other silkworms, bees, and bioengineered variants thereof. In some embodiments, silk fibroin can be extracted from a gland of silkworm or transgenic silkworms (see, e.g., WO 2007/098951).

The silk fibroin solution can be prepared by any conventional method known to one skilled in the art. For example, in one embodiment, B. mori cocoons are boiled for about 10 minutes to about 60 minutes (e.g., 30 minutes) in an aqueous solution. In one embodiment, the aqueous solution can comprise about 0.02M Na₂CO₃. The cocoons are rinsed, for example, with water to extract the sericin proteins and the extracted silk is dissolved in an aqueous salt solution. Salts useful for this purpose include lithium bromide, lithium thiocyanate, calcium nitrate or other chemicals capable of solubilizing silk. In some embodiments, the extracted silk is dissolved in about 8M-12 M LiBr solution. The salt is consequently removed using, for example, dialysis.

If necessary, the solution can then be concentrated using, for example, dialysis against a hygroscopic polymer, for example, PEG, a polyethylene oxide, amylose or sericin. In some embodiments, the PEG is of a molecular weight of 8,000-10,000 g/mol and has a concentration of 10%-50%. A slide-a-lyzer dialysis cassette (Pierce, MW CO 3500) can be used. However, any dialysis system may be used. The dialysis can be performed for a time period sufficient to result in a final concentration of aqueous silk solution between about 10%-about 30%. In most cases dialysis for 2-24 hours is sufficient. See, for example, International Application No. WO 2005/012606, the content of which is incorporated herein by reference.

Alternatively, the silk fibroin solution can be produced using organic solvents. Such methods have been described, for example, in Li, M., et al., J. Appl. Poly Sci. 2001, 79, 2192-2199; Min, S., et al. Sen'I Gakkaishi 1997, 54, 85-92; Nazarov, R. et al., Biomacromolecules 2004 May-June; 5(3):718-26. For example, an exemplary organic solvent that can be used to produce a silk solution includes, but is not limited to, hexafluoroisopropanol. See, for example, International Application No. WO/2004/000915, the content of which is incorporated herein by reference.

Depending on the desired mechanical property of a silk matrix, and/or release profile of a therapeutic agent from the silk matrix, different material states or forms of the silk matrix can be produced. For example, the silk matrix can be produced in a form of a hydrogel, a microneedle, a microparticle, a nanoparticle, a fiber, a film, lyophilized powder, a lyophilized gel, a reservoir implant, a homogenous implant, a tube, a gel-like or gel particle, and any combinations thereof. Accordingly, different concentrations of silk fibroin can be included in the silk matrix to achieve different material states or forms. Additional information on different forms of silk matrix and methods of making the same can be found, for example, in U.S. Pat. No. 8,187,616, the International Application No. WO 2005/012606, U.S. patent application Ser. Nos. 12/672,521, 12/442,595, 13/320,036, 13/254,629, 12/442,595, 12/974,796, 13/382,967, and Ser. No. 13/496,227, PCT Patent Application Serial Nos. PCT/US2010/050565, PCT/US2011/027153, PCT/US2011/056856, PCT/US2012/064139, PCT/US2012/064372, PCT/US2012/064471 and U.S. Provisional Application Nos. 61/621,209, 61/623,970, 61/613,185, the contents of which are incorporated herein by reference. In some embodiments, a silk matrix comprising silk fibroin can be produced from a silk solution containing silk fibroin at a concentration of about 0.1% (w/v) to about 30% (w/v), about 0.5% (w/v) to about 15% (w/v), about 1% (w/v) to about 8% (w/v), or about 1.5% (w/v) to about 5% (w/v). In some embodiments, a silk matrix comprising silk fibroin can be produced from a silk solution containing silk fibroin at a concentration of about 5% (w/v) to about 30% (w/v), about 10% (w/v) to about 25% (w/v), or about 15%(w/v) to about 20%(w/v).

In some embodiments, the silk matrix encapsulating a therapeutic agent can be in a form of a hydrogel. Various methods of producing silk hydrogel or silk fibroin hydrogel are known in the art. In some embodiments, the silk hydrogel can be produced by sonicating a silk solution containing a therapeutic agent and silk or silk fibroin at a concentration of about 0.25%(w/v) to about 30% (w/v), about 0.5%(w/v) to about 20%(w/v) or about 1% (w/v) to about 15% (w/v). In some embodiments, the silk solution can contain a therapeutic agent and silk or silk fibroin at a concentration that is not too viscous for injection, e.g., a silk concentration of about 0.5% (w/v) to about 10% (w/v). In one embodiment, the silk hydrogel can comprise silk fibroin at a concentration of about 1% (w/v) to about 10% (w/v), or about 1.5% (w/v) to about 3% (w/v). In one embodiment, the silk hydrogel can comprise silk fibroin at a concentration of about 2% (w/v) silk fibroin. See, e.g., U.S. Pat. App. No. U.S. 2010/0178304 and International App. No.: WO 2008/150861, the contents of which are incorporated herein by reference for methods of silk fibroin gelation using sonication.

In alternative embodiments, the silk hydrogel can be produced by applying a shear stress to a silk solution comprising a therapeutic agent and silk at a concentration of about 0.25% (w/v) to about 15% (w/v). See, e.g., International App. No.: WO 2011/005381, the content of which is incorporated herein by reference for methods of producing vortex-induced silk fibroin gelation for encapsulation and delivery. See, e.g., PCT Application Serial No. PCT/US2012/064372, the content of which is incorporated herein by reference, for examples of methods of applying shear stress to injectable silk fibroin particles.

In other embodiments, the silk hydrogel can be produced by modulating the pH of a silk solution comprising a therapeutic agent silk or silk fibroin at a concentration of about 0.25% (w/v) to about 15% (w/v). The pH of the silk solution can be altered by subjecting the silk solution to an electric field and/or reducing the pH of the silk solution with an acid. See, e.g., U.S. App. No.: US 2011/0171239, the content of which is incorporated herein by reference for details on methods of producing pH-induced silk gels.

In some embodiments where the silk hydrogel can have a high silk concentration, e.g., a concentration too high for injection through a small gauge needle (e.g., ˜27-˜30G), such as a silk or silk fibroin concentration of at least about 8% (w/v), at least about 10% (w/v), at least about 15% (w/v), at least about 20% (w/v), at least about 30% (w/v) or higher, the silk hydrogel can be reduced into gel-like or gel particles of any shape, e.g., spherical, rod, elliptical, cylindrical, capsule, or disc. The silk hydrogel can be reduced into gel-like or gel particles by any known methods in the art, e.g., grinding, cutting, and/or crushing. In some embodiments, the gel-like or gel particles can be of any size suitable for injection, e.g., a size of about 0.5 μm to about 2 mm, about 1 μm to about 1 mm, about 10 μm to about 0.5 mm, or about 50 μm to about 0.1 mm. In some embodiments, the gel-like or gel particles can have a size ranging from about 0.01 μm to about 1000 about 0.05 μm to about 500 about 0.1 μm to about 250 about 0.25 μm to about 200 or about 0.5 μm to about 100 μm.

In other embodiments, the silk matrix encapsulating a therapeutic agent can be in a form of a microparticle or nanoparticle. The microparticle or nanoparticle described herein can be of any shape, e.g., spherical, rod, elliptical, cylindrical, capsule, or disc. See, e.g., U.S. application Ser. Nos. 12/442,595, 13/496,227, and 13/582,903, U.S. Provisional Application No. 61/623,970, the contents of which are incorporated herein by reference, for examples of methods of generating silk microparticles and nanoparticles. The term “microparticle” as used herein refers to a particle having a particle size of about 0.01 μm to about 100 μm, about 0.05 μm to about 50 μm, about 0.1 μm to about 50 μm, about 0.25 μm to about 25 μm, or about 0.5 μm to about 15 μm. In one embodiment, the microparticle has a particle size of about 0.5 μm to about 15 μm. The term “nanoparticle” as used herein refers to particle having a particle size of about 0.5 nm to about 500 nm, about 1 nm to about 400 nm, about 10 nm to about 200 nm, about 25 nm to about 150 nm, or about 50 nm to about 100 nm. It will be understood by one of ordinary skill in the art that microparticles or nanoparticles usually exhibit a distribution of particle sizes around the indicated “size.” Unless otherwise stated, the term “size” as used herein refers to the mode of a size distribution of microparticles or nanoparticles, i.e., the value that occurs most frequently in the size distribution. Methods for measuring the microparticle or nanoparticle size are known to a skilled artisan, e.g., by dynamic light scattering (such as photocorrelation spectroscopy, laser diffraction, low-angle laser light scattering (LALLS), and medium-angle laser light scattering (MALLS)), light obscuration methods (such as Coulter analysis method), or other techniques (such as rheology, and light or electron microscopy).

Various methods of producing silk microparticles or nanoparticles are known in the art. In some embodiments, the silk microparticles or nanoparticles can be produced by a polyvinyl alcohol (PVA) phase separation method as described in, e.g., International App. No. WO 2011/041395, the content of which is incorporated herein by reference, for PVA methods of generating silk microparticles and/or nanoparticles. Other methods for producing silk microparticles or nanoparticles, e.g., described in U.S. App. No. U.S. 2010/0028451 and International App. No.: WO 2008/118133 (using lipid as a template for making silk microspheres or nanospheres), U.S. application Ser. No. 13/582,903 (using positively-charged and negatively-charged silk fibroin to form an ionomeric composition, e.g., particles) and in Wenk et al. J Control Release 2008; 132: 26-34 (using spraying method to produce silk microspheres or nanospheres) are incorporated herein by reference and can be used for the purpose of making silk microparticles or nanoparticles encapsulating a therapeutic agent.

In some embodiments, the silk microparticles, nanoparticles, or gel-like or gel particles can be further embedded in a solid substrate and/or a biomaterial, e.g., to prolong and/or localize the release of a therapeutic agent to a target site over a period of time. Examples of a solid substrate in which the silk microparticles, nanoparticles, or gel-like or gel particles can be embedded include, but are not limited to, a tablet, a capsule, a microchip, a hydrogel, a mat, a film, a fiber, an ocular delivery device, an implant, a coating, and any combinations thereof. See, e.g., U.S. application Ser. No. 13/254,629, the content of which is incorporated herein by reference, for exemplary methods of incorporating microparticles into silk hydrogels. In some embodiments, the silk microparticles can be incorporated into a silk hydrogel by conjugation methods known in the art, e.g., by covalent binding, e.g., as described in U.S. application Ser. No. 11/407,373, the content of which is incorporated herein by reference.

In some embodiments, the silk microparticles, nanoparticles, or gel-like or gel particles can be further embedded in a biomaterial or biopolymer, e.g., a biocompatible hydrogel. In some embodiments, the biopolymer can comprise a silk hydrogel, e.g., used to encapsulate the therapeutic agent-loaded silk microparticles, nanoparticles, or gel-like or gel particles. See, e.g., International App. No.: WO 2010/141133 for methods of producing silk fibroin scaffolds for antibiotic delivery.

In various embodiments, the silk matrix of any forms can be lyophilized or freeze-dried. In some embodiments, the process of lyophilization or freeze-drying can induce pore information in a silk matrix, e.g., as described in U.S. Pat. Nos. 7,842,780, and 8,361,617, the contents of which are incorporated herein by reference.

Optionally, the conformation of the silk fibroin in the silk matrix can be altered after formation of the silk matrix. Without wishing to be bound by a theory, the induced conformational change can alter the crystallinity of the silk fibroin in the silk matrix, e.g., silk II beta-sheet crystallinity. This can alter the rate of release of the therapeutic agent from the silk matrix. The conformational change can be induced by any methods known in the art, including, but not limited to, controlled slow drying (Lu et al., 10 Biomacromolecules 1032 (2009)), water annealing (Jin et al., Water-Stable Silk Films with Reduced β-Sheet Content, 15 Adv. Funct. Mats. 1241 (2005); Hu et al. Regulation of Silk Material Structure by Temperature-Controlled Water Vapor Annealing, 12 Biomacromolecules 1686 (2011)), stretching (Demura & Asakura, Immobilization of glucose oxidase with Bombyx mori silk fibroin by only stretching treatment and its application to glucose sensor, 33 Biotech & Bioengin. 598 (1989)), compressing, and solvent immersion, including methanol (Hofmann et al., Silk fibroin as an organic polymer for controlled drug delivery, 111 J Control Release. 219 (2006)), ethanol (Miyairi et al., Properties of b-glucosidase immobilized in sericin membrane. 56 J. Fermen. Tech. 303 (1978)), glutaraldehyde (Acharya et al., Performance evaluation of a silk protein-based matrix for the enzymatic conversion of tyrosine to L-DOPA. 3 Biotechnol J. 226 (2008)), and 1-ethyl-3-(3-dimethyl aminopropyl) carbodiimide (EDC) (Bayraktar et al., Silk fibroin as a novel coating material for controlled release of theophylline. 60 Eur J Pharm Biopharm. 373 (2005)); pH adjustment, e.g., pH titration and/or exposing a silk matrix to an electric field (see, e.g., U.S. Patent App. No. US2011/0171239, the content of which is incorporated herein by reference), heat treatment, shear stress (see, e.g., International App. No.: WO 2011/005381, the content of which is incorporated herein by reference), ultrasound, e.g., sonication (see, e.g., U.S. Pat. App. No. U.S. 2010/0178304 and International App. No.: WO 2008/150861, the contents of which are incorporated herein by reference), and any combinations thereof. In some embodiments, the silk matrix can comprise a silk II beta-sheet crystallinity content of at least about 5%, for example, a silk II beta-sheet crystallinity content of at least about 10%, at least about 20%, at least about 30%, at least about 40%, at least about 50%, at least about 60%, at least about 70%, at least about 80%, at least about 90%, or at least about 95% but not 100% (i.e. not where all the silk is present in a silk II beta-sheet conformation). In some embodiments, the silk in the silk matrix is present completely in a silk II beta-sheet conformation.

In some embodiments, the conformation of the silk fibroin in the silk matrix can be altered, e.g., by water annealing. For example, a non-crosslinked silk matrix comprising at least one therapeutic agent can be subjected to water annealing, e.g., to induce beta-sheet formation in silk fibroin.

In various embodiments, the silk fibroin can be modified for different applications and/or desired mechanical or chemical properties (e.g., to facilitate formation of a gradient of a therapeutic agent in silk fibroin matrices). One of skill in the art can select appropriate methods to modify silk fibroin, e.g., depending on the side groups of the silk fibroin, desired reactivity of the silk fibroin and/or desired charge density on the silk fibroin. In one embodiment, modification of silk fibroin can use the amino acid side chain chemistry, such as chemical modifications through covalent bonding, or modifications through charge-charge interaction. Exemplary chemical modification methods include, but are not limited to, carbodiimide coupling reaction (see, e.g. U.S. Patent Application. No. US 2007/0212730), diazonium coupling reaction (see, e.g., U.S. Patent Application No. US 2009/0232963), avidin-biotin interaction (see, e.g., International Application No.: WO 2011/011347) and pegylation with a chemically active or activated derivatives of the PEG polymer (see, e.g., International Application No. WO 2010/057142). Silk fibroin can also be modified through gene modification to alter functionalities of the silk protein (see, e.g., International Application No. WO 2011/006133). For instance, the silk fibroin can be genetically modified, which can provide for further modification of the silk such as the inclusion of a fusion polypeptide comprising a fibrous protein domain and a mineralization domain, which can be used to form an organic-inorganic composite. See WO 2006/076711. In some embodiments, the silk fibroin can be genetically modified to be fused with a protein, e.g., a therapeutic protein. Additionally, the silk fibroin matrix can be combined with a chemical, such as glycerol, that, e.g., affects flexibility and/or solubility of the matrix. See, e.g., WO 2010/042798, Modified Silk films Containing Glycerol.

In some embodiments, at least a portion of the silk matrix can further comprise at least one biocompatible polymer, including at least two biocompatible polymers, at least three biocompatible polymers or more. For example, a silk matrix can comprise one or more biocompatible polymers in a total amount of about 0.5 wt % to about 70 wt %, about 5 wt % to about 60 wt %, about 10 wt % to about 50 wt %, about 15 wt % to about 45 wt % or about 20 wt % to about 40 wt %, of the total silk matrix. In some embodiments, the biocompatible polymer(s) can be integrated homogenously or heterogeneously with the bulk of the silk matrix. In other embodiments, the biocompatible polymer(s) can be coated on a surface of the silk matrix. In any embodiments, the biocompatible polymer(s) can be covalently or non-covalently linked to silk in a silk matrix. In some embodiments, the biocompatible polymer(s) can be blended with silk within a silk matrix. Exemplary biocompatible polymers include, but are not limited to, a poly-lactic acid (PLA), poly-glycolic acid (PGA), poly-lactide-co-glycolide (PLGA), polyesters, poly(ortho ester), poly(phosphazine), poly(phosphate ester), polycaprolactone, gelatin, collagen, fibronectin, keratin, polyaspartic acid, alginate, cellulose, chitosan, chitin, hyaluronic acid, pectin, polyhydroxyalkanoates, dextrans, and polyanhydrides, polyethylene oxide (PEO), poly(ethylene glycol) (PEG), triblock copolymers, polylysine, any derivatives thereof and any combinations thereof.

Other additives suitable for use in some embodiments of the compositions described herein include biologically or pharmaceutically active compounds. Examples of biologically active compounds include, but are not limited to: cell attachment mediators, such as collagen, elastin, fibronectin, vitronectin, laminin, proteoglycans, or peptides containing known integrin binding domains e.g. “RGD” integrin binding sequence, or variations thereof, that are known to affect cellular attachment (Schaffner P & Dard 2003 Cell Mol Life Sci. January; 60(1):119-32; Hersel U. et al. 2003 Biomaterials. November; 24(24):4385-415); biologically active ligands; and substances that enhance or exclude particular varieties of cellular or tissue ingrowth. Other examples of additive agents that enhance proliferation or differentiation include, but are not limited to, osteoinductive substances, such as bone morphogenic proteins (BMP); cytokines, growth factors such as epidermal growth factor (EGF), platelet-derived growth factor (PDGF), insulin-like growth factor (IGF-I and II) TGF-β1. As used herein, the term additive also encompasses antibodies, DNA, RNA, modified RNA/protein composites, glycogens or other sugars, and alcohols.

Additionally, the silk matrix (e.g., silk microparticles, nanoparticles or gel-like or gel particles), and/or the composition described herein can also comprise a targeting ligand. As used herein, the term “targeting ligand” refers to any material or substance which can promote targeting of the silk matrix or the composition to tissues and/or receptors in vivo and/or in vitro. The targeting ligand can be synthetic, semi-synthetic, or naturally-occurring. Materials or substances which can serve as targeting ligands include, for example, proteins, including antibodies, antibody fragments, hormones, hormone analogues, glycoproteins and lectins, peptides, polypeptides, amino acids, sugars, saccharides, including monosaccharides and polysaccharides, carbohydrates, vitamins, steroids, steroid analogs, hormones, cofactors, and genetic material, including nucleosides, nucleotides, nucleotide acid constructs, peptide nucleic acids (PNA), aptamers, and polynucleotides. Other targeting ligands that can be used for some embodiments of the compositions described herein can include cell adhesion molecules (CAM), among which are, for example, cytokines, integrins, cadherins, immunoglobulins and selectin. The silk matrix or silk-based composition (e.g., silk microparticles, nanoparticles or gel-like or gel particles) can also encompass precursor targeting ligands. A precursor to a targeting ligand refers to any material or substance which can be converted to a targeting ligand. Such conversion can involve, for example, anchoring a precursor to a targeting ligand. Exemplary targeting precursor moieties include maleimide groups, disulfide groups, such as ortho-pyridyl disulfide, vinylsulfone groups, and azide groups. The targeting ligand can be covalently (e.g., cross-linked) or non-covalently linked to the silk matrix or silk-based composition. For example, a targeting ligand can be covalently linked to silk fibroin used for making the silk matrix.

In some embodiments, the silk matrix, e.g., microparticles, nanoparticles, gel-like or gel particles or implants, can be porous, wherein the silk matrix can have a porosity of at least about 30%, at least about 40%, at least about 50%, at least about 60%, at least about 70%, at least about 80%, at least about 90%, or higher. Too high porosity can yield a silk matrix with lower mechanical properties, but with faster release of a therapeutic agent. However, too low porosity can decrease the release of a therapeutic agent. One of skill in the art can adjust the porosity accordingly, based on a number of factors such as, but not limited to, desired release rates, molecular size and/or diffusion coefficient of the therapeutic agent, and/or concentrations and/or amounts of silk fibroin in a silk matrix. The term “porosity” as used herein is a measure of void spaces in a material, e.g., a matrix such as silk fibroin, and is a fraction of volume of voids over the total volume, as a percentage between 0 and 100% (or between 0 and 1). Determination of matrix porosity is well known to a skilled artisan, e.g., using standardized techniques, such as mercury porosimetry and gas adsorption, e.g., nitrogen adsorption.

The porous silk matrix can have any pore size. In some embodiments, the pores of a silk matrix can have a size distribution ranging from about 50 nm to about 1000 from about 250 nm to about 500 from about 500 nm to about 250 from about 1 μm to about 200 from about 10 μm to about 150 or from about 50 μm to about 100 μm. As used herein, the term “pore size” refers to a diameter or an effective diameter of the cross-sections of the pores. The term “pore size” can also refer to an average diameter or an average effective diameter of the cross-sections of the pores, based on the measurements of a plurality of pores. The effective diameter of a cross-section that is not circular equals the diameter of a circular cross-section that has the same cross-sectional area as that of the non-circular cross-section. In some embodiments, the silk fibroin can be swellable when the silk fibroin scaffold is hydrated. The sizes of the pores can then change depending on the water content in the silk fibroin. The pores can be filled with a fluid such as water or air.

Methods for forming pores in a silk matrix are known in the art, e.g., porogen-leaching method, freeze-drying method, and/or gas-forming method. Such methods are described, e.g., in U.S. Pat. App. Nos.: US 2010/0279112, US 2010/0279112, and U.S. Pat. No. 7,842,780, the contents of which are incorporated herein by reference.

Accordingly, any desirable release rates or release profiles of a therapeutic agent from a silk matrix can be, at least partly, adjusted by varying silk processing methods, e.g., concentration of silk in a silk matrix, amount of silk fibroin and/or beta-sheet conformation structures in a silk matrix, porosity and/or pore sizes of the silk matrix, and any combinations thereof.

In addition, silk matrix can stabilize the bioactivity of a therapeutic agent under a certain condition, e.g., under an in vivo physiological condition. See, e.g., the International Appl. Pub. No. WO/2012/145739, the content of which is incorporated herein by reference, for additional details on compositions and methods of stabilization of active agents. Accordingly, in some embodiments, encapsulating a therapeutic agent in a silk matrix can increase the in vivo half-life of the therapeutic agent. For example, in vivo half-life of a therapeutic agent dispersed or encapsulated in a silk matrix can be increased by at least about 5%, at least about 10%, at least about 15%, at least about 20%, at least about 30%, at least about 40%, at least about 50%, at least about 60%, at least about 90%, at least about 1-fold, at least about 1.5-folds relative to the therapeutic agent present in a non-silk matrix. Without wishing to be bound by theory, an increase in in vivo half-life of a therapeutic agent dispersed or encapsulated in a silk matrix can provide a longer therapeutic effect. Stated another way, an increase in in vivo half-life of a therapeutic agent dispersed or encapsulated in a silk matrix can allow loading of a smaller amount of the therapeutic agent for the same duration of therapeutic effect.

### Exemplary Therapeutic Agents

Generally, any therapeutic agent can be encapsulated in the silk matrix. As used herein, the term “therapeutic agent” generally means a molecule, group of molecules, complex or substance administered to an organism for diagnostic, therapeutic, preventative medical, or veterinary purposes. As used herein, the term “therapeutic agent” includes a “drug” or a “vaccine.” This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term can also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA), ribonucleic acid (RNA), or mixtures or combinations thereof, including, for example, DNA nanoplexes.

The term “therapeutic agent” also includes an agent that is capable of providing a local or systemic biological, physiological, or therapeutic effect in the biological system to which it is applied. For example, the therapeutic agent can act to control infection or inflammation, enhance cell growth and tissue regeneration, control tumor growth, act as an analgesic, promote anti-cell attachment, and enhance bone growth, among other functions. In some embodiments, the therapeutic agent can act to inhibit proliferation of abnormal blood vessels and/or induce regression of abnormal blood vessels. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism. Additionally, a silk-based composition can contain combinations of two or more therapeutic agents.

In some embodiments, different types of therapeutic agents that can be encapsulated or dispersed in a silk matrix can include, but not limited to, proteins, peptides, antigens, immunogens, vaccines, antibodies or portions thereof, antibody-like molecules, enzymes, nucleic acids, siRNA, shRNA, aptamers, small molecules, antibiotics, and any combinations thereof.

Exemplary therapeutic agents include, but are not limited to, those found in Harrison's Principles of Internal Medicine, 13^{th} Edition, Eds. T. R. Harrison et al. McGraw-Hill N.Y., NY; Physicians Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990, the complete contents of all of which are incorporated herein by reference.

In some embodiments, examples of therapeutic agents that can be dispersed or encapsulated in a silk matrix for ocular administration can include, but are not limited to, anti-inflammatory agents, anti-infective agents (including antibacterial, antifungal, antiviral, antiprotozoal agents), anti-allergic agents, anti-proliferative agents, anti-angiogenic agents, anti-oxidants, neuroprotective agents, cell receptor agonists, cell receptor antagonists, immunomodulating agents, immunosuppressive agents, intraocular pressure (IOP)-lowering agents (anti-glaucoma), beta adrenoceptor antagonists, alpha-2 adrenoceptor agonists, carbonic anhydrase inhibitors, cholinergic agonists, prostaglandins and prostaglandin receptor agonists, AMPA receptor antagonists, NMDA antagonists, angiotensin receptor antagonists, somatostatin agonists, mast cell degranulation inhibitors, alpha-2 adrenoceptor antagonists, thromboxane A2 mimetics, protein kinase inhibitors, prostaglandin F derivatives, prostaglandin-2 alpha antagonists and muscarinic agents.

In some embodiments, the therapeutic agent that can be dispersed or encapsulated in a silk matrix for ocular administration can include, but is not limited to, an agent for treatment of an ocular condition including, but not limited to, a posterior-segment disease or disorder. Additionally, any agent for treatment of any ocular condition noted below can be dispersed or encapsulated in a silk matrix for ocular administration, and/or used in the methods described herein. Examples of therapeutic agents for treatment of an ocular condition can include, without limitations, bevacizumab (e.g., AVASTIN®, Genentech), ranibizumab (LUCENTIS®, Genentech), aflibercept (EYLEA™, Regeneron Pharmaceuticals), pegaptanib (MACUGEN®, Eyetech, Inc.), tivozanib (e.g., AV-951, AVEO Pharmaceuticals), verteporfin (VISUDYNE®), fluocinolone acetonide (RETISERT®, Bausch & Lomb Incorporated), ganciclovir (e.g., VITRASERT®, Bausch & Lomb Incorporated), triamcinolone acetonide (e.g., TRIVARIS® Intravitreal or KENALOG®), foscarnet (e.g., FOSCAVIR®), dapiprazole (e.g., REV-EYES®), vancomycin, ceftazidime, amikacin, amphotericin B, dexamethasone, and any combinations thereof

In some embodiments, the therapeutic agent that can be dispersed or encapsulated in a silk matrix for ocular administration can include an agent for treatment of glaucoma, e.g., without limitations, travoprost, dorzolamide, timolol, bimatoprost, latanoprost, brimonidine, levobunolol, levobetaxolol, betaxolol, carbachol, epinephrine, pilocarpine, physostigmine, demecarium bromide, apraclonide, pilocarpine, acetylcholine, carteolol, metipranolol, echothiophate iodide, dipivefrin, unoprostone, and any combinations thereof.

In some embodiments, the therapeutic agent that can be dispersed or encapsulated in a silk matrix for ocular administration can include an agent for treatment of cytomegalovirus (CMV) retinitis, e.g., without limitations, valganciclovir, ganciclovir, foscarnet, cidofovir, fomivirsen, and any combinations thereof.

In some embodiments, the therapeutic agent that can be dispersed or encapsulated in a silk matrix for ocular administration can include an agent for treatment of macular degeneration including, e.g., age-related macular degeneration. Examples of such therapeutic agents can include, but are not limited to, bevacizumab (e.g., AVASTIN®; Genentech, Inc., South San Francisco, Calif.); ranibizumab (e.g., LUCENTIS®; Genentech, Inc., South San Francisco, Calif.); aflibercept (e.g., EYLEA™; Regeneron Pharmaceuticals, Tarrytown, N.Y.); pegaptanib (e.g., MACUGEN®; Eyetech, Inc.); tivozanib (e.g., AV-951, AVEO Pharmaceuticals, Cambridge, Mass.); verteporfin (e.g., VISUDYNE®; Novartis AG, Basel, Switzerland), and any anti-angiogenic agents known in the art.

Examples of anti-angiogenic agents can include, but are not limited to, VEGF inhibitors. Non-limiting examples of VEGF inhibitors can include bevacizumab (e.g., AVASTIN®; Genentech, Inc., South San Francisco, Calif.); ranibizumab (e.g., LUCENTIS®; Genentech, Inc., South San Francisco, Calif.); aflibercept (e.g., EYLEA™; Regeneron Pharmaceuticals, Tarrytown, N.Y.); pegaptanib (e.g., MACUGEN®; Eyetech, Inc.); tivozanib (e.g., AV-951, AVEO Pharmaceuticals, Cambridge, Mass.); verteporfin (e.g., VISUDYNE®; Novartis AG, Basel, Switzerland); 3-(4-Bromo-2,6-difluoro-benzyloxy)-5-[3-(4-pyrrolidin 1-yl-butyl)-ureido]-isothiazole-4-carboxylic acid amide hydrochloride (e.g., CP-547,632; Pfizer Inc., NY, NY); axitinib (e.g., AG13736; Pfizer, Inc., NY, NY); N,2-dimethyl-6-(2-(1-methyl-1H-imidazol-2-yl)thieno[3,2-b]pyridin-7-yloxy)benzo[b]thiophene-3-carboxamide (e.g., AG28262; Pfizer, Inc., NY, NY); N-(4-bromo-2-fluorophenyl)-6-methoxy-7-[(1-methylpiperidin-4-yl)methoxy]quinazolin-4-amine (e.g., ZD-6474, AstraZeneca); an inhibitor of VEGF-R2 and VEGF-R1 (e.g., ZD-4190; AstraZeneca); tyrosine kinase inhibitor of the RET/PTC oncogenic kinase (e.g., SU5416, SU11248 and SU6668; formerly Sugen Inc., now Pfizer, New York, New York); pan-VEGF-R-kinase inhibitor (e.g., CEP-7055; Cephalon Inc., Frazer, Pa.); protein kinase inhibitor (e.g., PKC 412; Novartis AG, Basel, Switzerland); multitargeted human epidermal receptor (HER) 1/2 and vascular endothelial growth factor receptor (VEGFR) 1/2 receptor family tyrosine kinases inhibitor (e.g., AEE788; Novartis AG, Basel, Switzerland); cediranib (e.g., AstraZeneca), sorafenib (e.g., NEXAVAR®, BAY 43-9006; Bayer AG, Barmen, Germany; and Onyx Pharmaceuticals, South San Francisco, Calif.); vatalanib (e.g., PTK-787, ZK-222584; Novartis AG, Basel, Switzerland; and Bayer Schering, Berlin-Wedding, Germany), anti-VEGF RNA aptamer (e.g., EYE-001; Pfizer Inc., NY, NY; Gilead, Foster City, Calif.; and Eyetech Inc.), glufanide disodium (e.g., IM862; Cytran Inc. of Kirkland, Wash.); VEGFR2-selective monoclonal antibody (e.g., DC101; ImClone Systems, Inc., East Bridgewater, N.J.); angiozyme, an siRNA-based VEGFR1 inhibitor, Fumagillin and analogue thereof (e.g., CAPLOSTATIN™), soluble ectodomains of the VEGF receptors, shark cartilage and derivatives thereof (e.g., NEOVASTAT™; AEterna Zentaris Inc., Quebec City, Canada); 5-((7-Benzyloxyquinazolin-4-yl)amino)-4-fluoro-2-methyl phenol hydrochloride (e.g., ZM323881; supplied from CalBiochem), any derivatives thereof and any combinations thereof.

In one embodiment, the VEGF inhibitor that can be dispersed or encapsulated in a silk matrix, e.g., for treatment of an angiogenesis-induced eye disease or disorder such as age-related macular degeneration can comprise bevacizumab (e.g., AVASTIN®; Genentech, Inc., South San Francisco, Calif.); ranibizumab (e.g., LUCENTIS®; Genentech, Inc., South San Francisco, Calif.), or a combination thereof.

In some embodiments, the therapeutic agent that can be dispersed or encapsulated in a silk matrix for ocular administration can comprise an agent for treatment of inflammation in an eye, e.g., caused by inflammation post surgery or due to injury. Examples such therapeutic agents can include, but are not limited to, nepafenac, ketorolac, cyclosporine, bromfenac, flurbiprofen, suprofen, diclofenac, dexamethasone, fluocinolone, fluorometholone, difluprednate, prednisolone, loteprednol, medrysone, rimexolone, triamcinolone, and any combinations thereof.

In some embodiments, the therapeutic agent that can be dispersed or encapsulated in a silk matrix for ocular administration can comprise an agent for treatment of ophthalmic bacterial infection, e.g., without limitations, bacitracin, polymyxin, levofloxacin, neomycin, ciprofloxacin, ofloxacin, tobramycin, moxifloxacin, azithromycin, trimethoprim, gatifloxacin, besifloxacin, chloramphenicol, erythromycin, gentamycin, gramicidin, idoxuridine, natamycin, norfloxacin, oxytetracycline, phenylephrine, silver nitrate, sulfacetamide sodium, sulfisoxazole, trifluridine, vidarabine, and any combinations thereof.

### Compositions for Ocular Administration

The compositions for ocular administration described herein can be formulated for various target sites of administration in an eye, e.g., but not limited to, lens, sclera, conjunctiva, aqueous humor, ciliary muscle, and vitreous humor. In some embodiments, the composition can be formulated to be an injectable composition, e.g., for intravitreal administration.

In some embodiments, the composition described herein can be formulated to include one or more water-soluble or ophthalmically-acceptable carriers or excipients. Exemplary water-soluble or ophthalmically-acceptable carriers or excipients can generally include sugars, saccharides, polysaccharides, surfactants, buffered solution, viscosity agents, and any combinations thereof. A non-limiting example of an excipient is 2-(hydroxymethyl)-6-[3,4,5-trihydroxy-6-(hydroxymethyl)tetrahydropyran-2-yl]oxy-tetrahydropyran-3,4,5-triol, “trehalose.” In some embodiments, the composition described herein can comprise about 1 wt. % to about 50 wt. % trehalose, or about 5 wt % to about 35 wt % trehalose, based on the weight of trehalose in the starting composition.

In some embodiments, the excipient can comprise one or more surfactants, including, for example and without limitation, polysorbate 20, polysorbate 80, and a combination thereof. For example, the composition can comprise from about 0.01 wt % to about 5 wt % polysorbate 20, or from about 0.05 wt % to about 0.25 wt % based on the weight of polysorbate 20 in the starting composition.

In some embodiments, the excipient can comprise one or more viscosity agents, including, for example and without limitation, hydroxypropyl methylcellulose (HPMC), hyaluronic acid, and the like.

In some embodiments, a viscosity-modulating component can be present in an effective amount in modulating the viscosity of the composition. In some embodiments, increasing the viscosity of the compositions to values in excess of the viscosity of water (1 centipoise) can allow more effective placement, e.g., injection, of the composition into the posterior segment of an eye. In other embodiments, a viscosity-modulating component can include a shear thinning component, which, when present in the composition, can reduce the viscosity of the composition under a high shear condition as the composition is passed through a narrow space, such as a 27-gauge needle, and injected into the posterior segment of an eye, but the composition can regain its pre-injection viscosity after the passage through the injection needle.

Any suitable viscosity-modulating component, for example, ophthalmically acceptable viscosity-modulating component, can be employed in some embodiments of the compositions described herein. Examples of viscosity-modulating components can include, but are not limited to, hyaluronic acid (such as a polymeric hyaluronic acid), carbomers, polyacrylic acid, cellulosic derivatives, polycarbophil, polyvinylpyrrolidone, gelatin, dextrin, polysaccharides, polyacrylamide, polyvinyl alcohol, polyvinyl acetate, derivatives thereof and mixtures and copolymers thereof. The specific amount of the viscosity-modulating component employed in the composition can depend upon a number of factors including, for example and without limitation, the specific viscosity-modulating component being employed, the molecular weight of the viscosity-modulating component being employed, the viscosity desired for the composition, shear thinning, biocompatibility and/or biodegradability of the compositions. In some embodiments, the viscosity-modulating component can be present in an amount in a range of about 0.5% or about 1.0% to about 5% or about 10% or about 20% (w/v) of the composition.

In some embodiments, the composition can comprise at least one buffer component in an amount effective to control and/or maintain the pH of the composition. In some embodiments, the amount of the buffer component employed can be sufficient to maintain the pH of the composition in a range of about 6 to about 8, or about 7 to about 7.5. The buffer component can be chosen from those which are known in the ophthalmic art. Examples of such buffer components include, but are not limited to, acetate buffers, citrate buffers, phosphate buffers, borate buffers and mixtures thereof. In some embodiments, the buffer component can comprise a phosphate buffer.

In some embodiments, the composition can comprise at least one tonicity component in an amount effective to control the tonicity or osmolality of the composition. In some embodiments, the amount of tonicity component employed can be sufficient to provide an osmolality to the composition described herein in a range of about 200 to about 400 mOsmol/kg, or about 250 to about 350 mOsmol/kg, respectively. In one embodiment, the tonicity and/or osmolality of the composition is adjusted to be substantially isotonic to the vitreous humor. The tonicity component can be chosen from those which are known in the ophthalmic art. Suitable tonicity components can include, but are not limited to, salts, e.g., sodium chloride, potassium chloride, mannitol and other sugar alcohols, and other suitable ophthalmically acceptably tonicity component and mixtures thereof.

The compositions described herein can be sterilized using conventional sterilization process such as radiation based sterilization (i.e. gamma-ray), chemical based sterilization (ethylene oxide), autoclaving, or other appropriate procedures. In some embodiments, sterilization process can be with ethylene oxide at a temperature between from about 52° C. to about 55° C. for a time of 8 or less hours. After sterilization, the composition can be packaged in an appropriate sterilized moisture-resistant package for storage and/or transport.

### Methods of Using One or More Embodiments of the Compositions Described Herein (Including, e.g., Methods of Treatment)

Different embodiments of the compositions, ocular delivery devices and/or kits described herein can be used for delivering a therapeutic agent to an eye, e.g., to treat an ocular condition, e.g., an angiogenesis-induced ocular disease or disorder such as age-related ocular condition. Accordingly, another aspect provided herein relates to a method for delivering a therapeutic agent to an eye of a subject. The method comprises administering to a target site of an eye a therapeutic agent dispersed or encapsulated in a silk matrix, wherein an amount of the therapeutic agent dispersed or encapsulated in the silk matrix can provide a therapeutic effect for a period of time which is longer than when the same amount of the therapeutic agent is administered without the silk matrix.

The inventors have demonstrated that a therapeutic agent dispersed or encapsulated in a silk matrix can reduce a likelihood of the therapeutic agent leaking from a target administration site at the eye, as compared to the same therapeutic agent administered without the silk matrix. Accordingly, further provided herein is a method for increasing an effective amount of a therapeutic agent administered to a target site of an eye. Such method comprises administering to a target site of an eye of a subject a therapeutic agent dispersed or encapsulated in a silk matrix, wherein the silk matrix is formulated to reduce leakage of the therapeutic agent from the target administration site, thereby increasing the actual amount of the therapeutic agent delivered to the target site of the eye and/or close proximity thereof. Thus, an effective and/or actual amount of the therapeutic agent delivered to a target site of an eye using one or more embodiments of the compositions and/or methods described herein can be increased by at least about 1%, at least about 2%, at least about 5%, at least about 10%, at least about 20%, at least about 30%, at least about 40%, at least about 50% or more, as compared to the same therapeutic agent administered without the silk matrix.

As used herein, the term “effective amount” refers to an actual amount of a therapeutic agent that is delivered to at least one cell at a target site of an eye such that a desired effect is produced. For example, when a therapeutic agent can leak from an injection needle or from an injection site after withdrawing the injection needle upon administration, the actual amount of the therapeutic agent that can be delivered to at least one cell at a target site of an eye for a desired effect can be at least about 1% smaller than the initial pre-determined amount for administration, e.g., at least about 2%, at least about 5%, at least about 10%, at least about 20%, at least about 30%, at least about 40%, at least about 50% or more, smaller than the initial pre-determined amount for administration.

As used herein, the term “administer” or “administering” refers to the placement of a composition into a subject by a method or route which results in at least partial localization of the composition at a desired site such that desired effect is produced. Routes of administration suitable for the methods described herein can include both local and systemic administration. Generally, local administration results in more of the therapeutic agent being delivered to a specific location as compared to the entire body of the subject, whereas, systemic administration results in delivery of the therapeutic agent to essentially the entire body of the subject. In particular embodiments, administration of a composition described herein or a therapeutic agent encapsulated in a silk matrix can encompass placing the composition or the therapeutic agent encapsulated in a silk matrix into a target site of a subject's eye or at least a portion of a subject's eye, e.g., but not limited to, lens, sclera, conjunctiva, aqueous humor, ciliary muscle, vitreous humor, or any combinations thereof. In one embodiment, administration of a composition described herein or a therapeutic agent encapsulated in a silk matrix can encompass placing the composition or the therapeutic agent encapsulated in a silk matrix into the vitreous humor of a subject's eye. In one embodiment, administration of a composition described herein or a therapeutic agent encapsulated in a silk matrix to an eye (e.g., vitreous humor of an eye) can be performed by injection.

Methods of Treatment:

As noted earlier, the inventors have discovered that the therapeutic agent dispersed or encapsulated in a silk matrix can unexpectedly provide a therapeutic effect over a longer period of time, e.g., at least one week longer, or at least one month longer, than the duration of the therapeutic effect when the same amount of the therapeutic agent is administered without the silk matrix. Thus, when a subject is administered with one or more embodiments of the compositions described herein, the frequency of administering a subject with a composition described herein can be reduced, when compared to the same amount of the therapeutic agent is administered to the subject without the silk matrix.

Accordingly, a still another aspect provided herein relates to methods for administrating a therapeutic agent to a target site of an eye of a subject in need thereof, which comprises administrating to a target site of an eye of a subject one or more embodiments of the composition described herein at a frequency, which is less than the administration frequency when the same amount of the therapeutic agent is administered without the silk matrix. In some embodiments, the frequency of administration (F) using one or more embodiments of the composition described herein can be reduced by a factor of F=(Y2−Y1)/Y2, wherein Y1 is the duration of the therapeutic effect produced by the current dosage of the therapeutic agent without silk matrix recommended for a particular indication, and Y2 is the duration of the therapeutic effect produced by the same amount of the therapeutic agent present in a silk matrix described herein. For example, the duration of the therapeutic effect produced by the current recommended dosage of the therapeutic agent, e.g., AVASTIN® in non-silk solution, for treatment of AMD is about 1 month, while the duration of the therapeutic effect can be extended to about 2 months when the same amount of the therapeutic agent, e.g., AVASTIN®, is administered in silk matrix. Thus, the frequency of administration can be reduced by a factor of (2−1)/2=½. That is, instead of having an administration of AVASTIN® once a month with the current administration protocol, the methods and/or compositions described herein can reduce frequency of administration to about once every two months (i.e., 1 injection/month*(1−F)). Similarly, if the frequency of administration is reduced by a factor of ⅔ (e.g., Y2=3 months, and Y1=1 month), the methods and/or compositions described herein can reduce frequency of administration to about once every 3 months.

In some embodiments, the frequency of administration of a therapeutic agent can be reduced by a factor of at least about ⅕, at least about ¼, at least about ⅓, at least about ½ or more. In some embodiments, the frequency of administration of a VEGF inhibitor (e.g., bevacizumab) can be reduced by a factor of at least about ⅕, at least about ¼, at least about ⅓, at least about ½ or more.

In a further aspect, provided herein is a method for treating an ocular condition in a subject, which comprises administering to a target site of an eye of a subject one or more embodiments of the composition described herein. In some embodiments, the composition can provide a sustained release of the therapeutic agent to the target site of the eye and/or close proximity thereof, thereby treating the ocular condition in the subject.

The term “therapeutic effect” as used herein refers to a consequence of treatment, the results of which are judged to be desirable and beneficial. In some embodiments, the therapeutic effect is associated with treatment of an ocular condition. The terms “treatment” and “treating” as used herein, with respect to treatment of a disease, means preventing the progression of the disease, or altering the course of the disorder (for example, but are not limited to, slowing the progression of the disorder), or reversing a symptom of the disorder or reducing one or more symptoms and/or one or more biochemical markers in a subject, preventing one or more symptoms from worsening or progressing, promoting recovery or improving prognosis. For example, in the case of treating an ocular condition such as an angiogenesis-induced ocular condition, e.g., age-related macular degeneration, therapeutic treatment refers to regression of the abnormal blood vessels and/or improvement of vision after administration of the composition described herein. In another embodiment, the therapeutic treatment refers to alleviation of at least one symptom associated with an ocular condition such as an angiogenesis-induced ocular condition, e.g., age-related macular degeneration. Measurable lessening includes any statistically significant decline in a measurable symptom, such as reduced growth of abnormal blood vessels and/or improved vision after treatment. In one embodiment, at least one symptom of an ocular condition such as an angiogenesis-induced ocular condition, e.g., age-related macular degeneration, is alleviated by at least about 10%, at least about 15%, at least about 20%, at least about 30%, at least about 40%, or at least about 50%. In another embodiment, at least one symptom is alleviated by more than 50%, e.g., at least about 60%, at least about 70% or higher (but excluding 100%), as compared to a control (e.g., in the absence of the composition described herein). In one embodiment, at least one symptom is alleviated by at least about 80%, at least about 90% or greater (but excluding 100%), as compared to a control (e.g. in the absence of the composition described herein). Accordingly, in some embodiments, the therapeutic effect can be determined by a reduction of at least one symptom associated with the ocular condition, such as improved vision or regression of abnormal blood vessels, by at least about 10%, at least about 15%, at least about 20%, at least about 30%, at least about 40%, or at least about 50% (but excluding 100%), as compared to a control (e.g. in the absence of the composition described herein). In another embodiment, at least one symptom is alleviated by more than 50%, e.g., at least about 60%, or at least about 70% (but excluding 100%), as compared to a control (e.g. in the absence of the composition described herein). In one embodiment, at least one symptom is alleviated by at least about 80%, at least about 90% or greater (but excluding 100%), as compared to a control (e.g. in the absence of the composition described herein). In some embodiments, the therapeutic effect produced by any aspects of the methods described herein can sustain for a period of time, which is at least about 1 week longer than when the same amount of the therapeutic agent is administered without the silk matrix. In some embodiments, the therapeutic effect produced by any aspects of the methods described herein, e.g., determined by a reduction of at least one symptom associated with the ocular condition, such as improved vision or regression of abnormal blood vessels by at least about 10%, can sustain for a period of time, which is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months or more, longer than when the same amount of the therapeutic agent is administered without the silk matrix.

In one embodiment, the ocular condition to be treated can be age-related macular degeneration. In such embodiments, the therapeutic agent dispersed or encapsulated in the silk matrix can comprise an angiogenesis inhibitor, e.g., a VEGF inhibitor. Exemplary VEGF inhibitors can include bevacizumab, ranibizumab, aflibercept, pegaptanib, tivozanib, and any combinations thereof. In one embodiment, provided herein is a method for treating age-related macular degeneration in a subject comprises administering to a target site of an eye of a subject (e.g., vitreous humor of an eye) a composition comprising bevacizumab, ranibizumab, or a combination thereof, dispersed or encapsulated in a silk matrix. In such embodiment, the amount of bevacizumab, ranibizumab, or a combination thereof, dispersed or encapsulated in the silk matrix can be substantially the same amount as the current recommended dosage in a non-silk solution formulation, for example, about 0.5 mg to about 1.5 mg (e.g., about 1.25 mg) for treatment of AMD, but the silk-based composition can provide a therapeutic effect over a period of time, which is at least about 1 week, at least about 1 month, at least about 2 months, at least about 3 months, at least about 4 months, at least about 5 months, at least about 6 months, longer than that provided by the recommended dosage of the existing non-silk solution formulation. In another embodiment, the amount of bevacizumab, ranibizumab, or a combination thereof, dispersed or encapsulated in the silk matrix can be in an amount higher (e.g., at least about 10% higher, at least about 20% higher, at least about 30% higher, at least about 40% higher, at least about 50% higher, at least about 60% higher, at least about 70% higher, at least about 80% higher, at least about 90% higher, at least about 1-fold higher, at least about 2-fold higher, at least about 3-fold higher, at least about 4-fold higher, at least about 5-fold higher, at least about 6-fold higher, at least about 7-fold higher, at least about 8-fold higher, at least about 9-fold higher, or at least about 10-fold higher) than what is allowed in the current recommended dosage (in non-silk solution formulation) for treatment of AMD. This embodiment can provide a therapeutic effect over a period of time, which is at least about 2 months, at least about 3 months, at least about 6 months, at least about 9 months, at least about 12 months, longer that that provided by the recommended dosage of the existing non-silk solution formulation. In alternative embodiments, the amount of bevacizumab, ranibizumab, or a combination thereof, dispersed or encapsulated in the silk matrix can be in an amount lower (e.g., at least about 5% lower, at least about 10% lower, at least about 20% lower, at least about 30% lower, at least about 40% lower, or at least about 50% lower) than the current recommended dosage (in non-silk solution formulation) for treatment of AMD. Such embodiment can provide a therapeutic effect over a substantially same period of time or even a longer period of time, e.g., at least about 1 week longer, including, e.g., at least about 2 weeks, at least 3 weeks, at least about 1 month, at least about 2 months, at least about 3 months, at least about 4 months, at least about 5 months, at least about 6 months longer, than that provided by the recommended dosage of the existing non-silk solution formulation.

Depending on the duration of the therapeutic effect produced by different embodiments of the composition, the frequency of administration of the composition to an eye of a subject can vary. In general, the longer the sustained release of the therapeutic agent to a target site, the less frequently the administration needs to be performed. In some embodiments, the composition can be administered to a target site of an eye of a subject at least every month, at least every two months, at least every three months, at least every four months, at least every five months, at least every six months, at least every seven months, at least every eight months, at least every nine months, at least every ten months, at least every eleven months, at least every twelve months or less frequently. Stated another way, in some embodiments of any aspects of the methods described herein, the administration can be performed no more than once a month, no more than once every two months, no more than once every three months, no more than once every four months, no more than once every five months, or no more once every six months or less frequently.

In any aspects of the methods described herein, the therapeutic agent dispersed or encapsulated in the silk matrix or the composition described herein can be administered to any part of an eye, e.g., the anterior segment of the eye, or the posterior segment of the eye. In some embodiments, the therapeutic agent dispersed or encapsulated in the silk matrix or the composition described herein can be administered to at least a portion of the eye selected from the group consisting of lens, sclera, conjunctiva, aqueous humor, ciliary muscle, and vitreous humor. In one embodiment, the therapeutic agent dispersed or encapsulated in the silk matrix or the composition described herein can be administered to the vitreous humor of the eye.

In any aspects of the methods described herein, the therapeutic agent dispersed or encapsulated in the silk matrix or the composition described herein can be administered to the eye by any methods known in the art, e.g., injection, topical (e.g., using an eye dropper, or a contact lens as a delivery device), implantation. In some embodiments, the therapeutic agent dispersed or encapsulated in the silk matrix or the composition described herein can be administered to the eye by injection, e.g., intravitreal injection. In one embodiment, the therapeutic agent dispersed or encapsulated, e.g., in a silk hydrogel, a microparticle or a nanoparticle, a gel-like or gel particle or any combinations thereof, can be administered by a non-invasive method, e.g., injection. The injection can performed with an injection needle suitable for eye injection, e.g., an injection needle with a gauge of about 25 to about 34, or about 27 to about 30. Other ocular delivery devices, e.g., intravitreal injection devices, known in the art can also be used for administration of the composition described herein, e.g., but not limited to, the ones described in U.S. Pat. App. Nos. US2010/0152646, US2010/0100054, US2010/0305514, US2006/0259008, and U.S. Pat. No. 7,678,078, the contents of which are incorporated herein by reference.

Diagnosis and/or monitoring of an ocular condition, e.g., an angiogenesis-induced ocular condition such as age-related macular degeneration, are known to a skilled practitioner. By way of example only, to detect age-related macular degeneration (AMD), an ophthalmic medical practitioner can perform an eye examination, which includes visual acuity test, dilated eye exam and/or tonometry. Visual acuity test is an eye chart test to measure how well a subject can see at various distances. In a dilated eye exam, an ophthalmic medical practitioner can use eye drops to dilate, or enlarge, a subject's pupil and then use a special magnifying lens to examine a subject's retina and optic nerve for signs of AMD and other eye problems. Tonometry is an instrument that measures the pressure inside the eye.

In some instances, an ophthalmic medical practitioner can ask a subject to look at an Amsler grid, the pattern of which resembles a checkerboard. A subject covers one eye and stares at a black dot in the center of the grid. While staring at the dot, if a subject can notice that the straight lines in the pattern may appear wavy, or some of the lines are missing, it can be indicative of a subject having an AMD or at risk of having an AMD.

An ophthalmic medical practitioner can also utilize fundus photography and angiography, optical coherence tomography, and/or ultrasound examination and ultrasound biomicroscopy to facilitate diagnosis of AMD or other ocular conditions and/or monitoring the progression of a treatment.

Digital fundus photography can be used to photograph any abnormalities in order to examine any change in the appearance of a patient's retina and macula over time. An angiogram is a type of photograph that can allow an ophthalmic medical practitioner to visualize more clearly the blood vessels in the back of a subject's eye as well as associated abnormalities, such as the growth of abnormal new blood vessels (neovascularization), the most common cause of vision loss in AMD. For example, an angiogram can be performed by taking photographs of the macula and retina after the injection of a food dye called fluorescein into a peripheral vein, generally in the patient's arm or hand. The dye can circulate through the blood vessels, including the eye, and can be eliminated from the body over a few days through the urine. In some instances, indocyanine green (ICG) angiography can supplement standard fluorescein angiography (FA).

Optical Coherence Tomography (OCT) is generally used by an ophthalmic medical practitioner to create cross-sectional images of the front or back of a patient's eye, similar to the images created by computed tomography (VAT′ or ‘CT scan’), to allow detailed examination of ocular structures in a patient. OCT imaging is generally a rapid, non-invasive test, similar to the experience of having a photograph of the retina, and thus can be performed in a clinic setting. OCT can be used in the diagnosis and monitoring of neovascular AMD over time. To follow the progress of a treatment, an ophthalmic medical practitioner can perform repeated measurements during the treatment.

Ophthalmic ultrasound is generally a non-invasive test and is generally used to diagnose eye pathology including tumors, especially when visualization to the interior structures is poor due to media opacities. Ophthalmic ultrasound and ultrasound biomicroscopy (UBM) has a very high resolution (2 to −60 microns) compared to conventional ultrasound (300 to 600 microns), allowing an ophthalmic medical practitioner to study anterior eye structures as if looking at a pathological specimen through a low power microscope.

### Selection of a Subject for Treatment

In some embodiments, subjects are selected for treatment prior to administering the compositions, ocular delivery device, or kits described herein or employing the methods described herein. In some embodiments, the subject can be diagnosed with having an ocular condition or at risk of having an ocular condition, prior to administering to the subject the compositions, ocular delivery device, or kits described herein or employing the methods described herein.

In some embodiments, the subject selected for treatment with one or more embodiments of the compositions, ocular delivery devices, kits, and/or methods described herein can be determined to have one or more symptoms associated with an ocular condition, prior to the administration. Examples of symptoms associated with an ocular condition can include, but are not limited to, e.g., but not limited to, presence of drusen; pigmentary alterations; exudative changes (e.g., hemorrhages in the eye, hard exudates, subretinal/sub-RPE/intraretinal fluid); visual acuity drastically decreasing (e.g., two levels or more such as 20/20 to 20/80); preferential hyperacuity perimetry changes; blurred vision; central scotomas (e.g., shadows or missing areas of vision); distorted vision in the form of metamorphopsia, for example, in which a grid of straight lines appears wavy and parts of the grid may appear blank; trouble discerning colors (specifically dark ones from dark ones and light ones from light ones); slow recovery of visual function after exposure to bright light; a loss in contrast sensitivity blurry; and any combinations thereof.

In some embodiments, the subject selected for treatment with one or more embodiments of the compositions, ocular delivery devices, kits, and/or methods described herein can be diagnosed with having, or having a risk for, age-related macular degeneration (AMD), prior to the administration.

In some embodiments, the subject selected for treatment with one or more embodiments of the compositions, ocular delivery devices, kits, and/or methods described herein can be diagnosed with having, or having a risk for, proliferation of abnormal growth vessels and/or increased intraocular pressure, prior to the administration.

In some embodiments, the subject selected for the methods described herein can be previously recovered from an ocular condition described herein (e.g., but not limited to AMD) and is diagnosed with recurrence of the ocular condition.

In other embodiments, the subject selected for the methods described herein can have undergone or is undergoing at least one other treatment for an ocular condition. For example, a subject diagnosed with age-related macular degeneration and being administered with an anti-VEGF inhibitor without the silk matrix (e.g., AVASTIN® and/or LUCENTIS®) can be selected for the methods described herein. Other treatments can include laser therapy, e.g., to destroy the abnormal blood vessels, and/or a photodynamic therapy, in which verteporfin is injected into a peripheral vein, such that verteporfin travels throughout the body and preferentially binds to the surface of new blood vessels, including the new blood vessels in the eye. Verteporfin can then be light-activated to destroy the new blood vessels.

As used herein, a “subject” can mean a human or an animal. Examples of subjects include primates (e.g., humans, and monkeys). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. A patient or a subject includes any subset of the foregoing, e.g., all of the above, or includes one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female. In some embodiments, a subject can be of any age, including infants.

In one embodiment, the subject is a mammal. The mammal can be a human, non-human primate, mouse, rat, dog, rabbit, cat, horse, or cow, but are not limited to these examples. Mammals other than humans can be advantageously used as subjects that represent animal models of treatment of an ocular condition. In addition, the methods and compositions described herein can be employed in domesticated animals and/or pets.

By an “ocular condition” is generally meant a disease, aliment or condition which can affect or involve the eye or at least one part or region of the eye, such as a retinal disease. The eye includes the eyeball and the tissues and fluids (which constitute the eyeball), the periocular muscles (such as the oblique and rectus muscles) and the portion of the optic nerve that is within or adjacent to the eyeball. An ocular condition can be any disease or disorder associated with any part of an eye. For example, the ocular condition can include, but are not limited to, age-related macular degeneration, choroidal neovascularization, diabetic macular edema, acute and chronic macular neuroretinopathy, central serous chorioretinopathy, macular edema, acute multifocal placoid pigment epitheliopathy, Behcet's disease, birdshot retinochoroidopathy, posterior uveitis, posterior scleritis, serpignous choroiditis, subretinal fibrosis, uveitis syndrome, Vogt-Koyanagi-Harada syndrome, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, carotid artery disease (CAD), frosted branch angitis, sickle cell retinopathy, angioid streaks, familial exudative vitreoretinopathy, Eales disease, proliferative vitreal retinopathy, diabetic retinopathy, retinal disease associated with tumors, congenital hypertrophy of the retinal pigment epithelium (RPE), posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors, myopic retinal degeneration, acute retinal pigment epithelitis, glaucoma, endophthalmitis, cytomegalovirus retinitis, retinal cancers, and any combinations thereof.

In some embodiments, an ocular condition can include a posterior ocular condition, which involve a posterior segment of an eye, such as choroid or sclera (in a position posterior to a plane through the posterior wall of the lens capsule), vitreous humor, vitreous chamber, retina, optic nerve (including the optic disc), and blood vessels and nerve which vascularize or innervate a posterior ocular region or site. Examples of posterior ocular conditions can include, but are not limited to, macular degeneration (such as non-exudative age related macular degeneration and exudative age related macular degeneration); macular hole; light, radiation or thermal damage to a posterior ocular tissue; choroidal neovascularization; acute macular neuroretinopathy; macular edema (such as cystoid macular edema and diabetic macular edema); Behcet's disease, retinal disorders, diabetic retinopathy (including proliferative diabetic retinopathy); retinal arterial occlusive disease; central retinal vein occlusion; uveitic retinal disease; retinal detachment; ocular trauma which affects a posterior ocular site; a posterior ocular condition caused by or influenced by an ocular laser treatment; posterior ocular conditions caused by or influenced by a photodynamic therapy; photocoagulation; radiation retinopathy; epiretinal membrane disorders; branch retinal vein occlusion; anterior ischemic optic neuropathy; non-retinopathy diabetic retinal dysfunction, retinitis pigmentosa and glaucoma. Glaucoma can be considered as a posterior ocular condition because the therapeutic goal is to prevent the loss of or reduce the occurrence of loss of vision due to damage to or loss of retinal cells or retinal ganglion cells (i.e. neuroprotection). In some embodiments, an ocular condition to be treated is age-related macular degeneration.

In other embodiments, an ocular condition can include an anterior ocular condition, which involves an anterior segment of an eye, such as a periocular muscle, an eye lid or an eye ball tissue or fluid which is located anterior to the posterior wall of the lens capsule or ciliary muscles. Thus, an anterior ocular condition can primarily affect or involve, the conjunctiva, the cornea, the conjunctiva, the anterior chamber, the iris, the posterior chamber (behind the iris but in front of the posterior wall of the lens capsule), the lens or the lens capsule and blood vessels and nerve which vascularize or innervate an anterior ocular region or site. Examples of an anterior ocular condition can includes, but are not limited to, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; hyperopia; pupil disorders; refractive disorders and strabismus. Glaucoma can also be considered as an anterior ocular condition because a clinical goal of glaucoma treatment can be to reduce a hypertension of aqueous fluid in the anterior chamber of the eye (i.e. reduce intraocular pressure).

### Ocular Delivery Devices and Kits

Ocular delivery devices and kits, e.g., to facilitate administering any embodiments of the compositions and/methods described herein are also provided herein. In some embodiments, an ocular delivery device can comprise one or more embodiments of the composition described herein. In some embodiments, one or more embodiments of the compositions described herein can be pre-loaded into the ocular delivery device. An ocular delivery device can exist in any form, e.g., in some embodiments, the device can be a syringe with an injection needle, e.g., having a gauge of about 25 to about 34 or of about 27 to about 30. Other examples of an ocular delivery device that can be used for administration of one or more embodiments of the compositions described herein and/or used in one or more embodiments of the methods described herein can include, but are not limited to, a contact lens, an eye-dropper, a microneedle (e.g., a silk microneedle), an implant, and any combinations thereof. Additional ocular delivery devices can be used to deliver some embodiments of the compositions described herein and/or be used in the methods described herein can include, but are not limited to, the ones described in U.S. Pat. App. Nos. US2010/0152646, US2010/0100054, US2010/0305514, US2006/0259008, US2006/0204548, and U.S. Pat. No. 7,678,078, the contents of which are incorporated herein by reference.

In one embodiment, one or more embodiments of the compositions comprising a therapeutic agent dispersed or encapsulated in a silk matrix can be pre-loaded into a syringe, optionally attached to an injection needle. In contrast to current practice, where an anti-VEGF inhibitor, e.g., bevacizumab, requires reconstitution in solution prior to loading into a syringe for injection by a physician on site, a therapeutic agent dispersed or encapsulated into a silk matrix can maintain its bioactivity, e.g., even at room temperature, and can thus be pre-loaded into a syringe, optionally attached to an injection needle, for the “off-the-shelf” applications.

In any embodiment of the ocular delivery device, the therapeutic agent dispersed or encapsulated in a silk matrix can vary with desirable administration schedule, and/or release profiles of the therapeutic agent. For example, the therapeutic agent can be present in a silk matrix in an amount sufficient to maintain a therapeutically effective amount thereof delivered to at least a portion of an eye, upon administration, over a period of more than 1 month, including, e.g., more than 2 months, more than 3 months, more than 4 months, more than 5 months, more than 6 months, more than 9 months, more than 12 months or longer. In general, the longer the sustained release of the therapeutic agent to a target site, the less frequently the administration needs to be performed. In some embodiments, the therapeutic agent or the VEGF inhibitor can be present in a silk matrix in an amount of about 0.01 mg to about 50 mg, or about 5 mg to about 10 mg. Amounts or dosages of the therapeutic agent encapsulated or dispersed in a silk matrix as described in any embodiment of the compositions described herein can be applicable to any embodiment of the ocular delivery device described herein.

In one embodiment, the ocular delivery device comprises a VEGF inhibitor (e.g., bevacizumab, ranibizumab, or a combination thereof) encapsulated in a silk matrix, wherein about 0.5 mg to about 1.5 mg (e.g., about 1.25 mg) of the VEGF inhibitor (e.g., bevacizumab, ranibizumab, or a combination thereof) encapsulated in the silk matrix provides a therapeutic effect for at least about 2 months, at least about 3 months, or longer.

In one embodiment, the ocular delivery device comprises a VEGF inhibitor (e.g., bevacizumab, ranibizumab, or a combination thereof) encapsulated in a silk matrix, wherein about 1.5 mg to about 10 mg of the VEGF inhibitor (e.g., bevacizumab, ranibizumab, or a combination thereof) encapsulated in the silk matrix provides a therapeutic effect for at least about 3 months, at least about 4 months, at least about 5 months, at least about 6 months, at least about 9 months, at least about 12 months, at least about 18 months, at least about 24 months, or longer. In one embodiment, about 3 mg to about 10 mg (e.g., about 5 mg) of the VEGF inhibitor (e.g., bevacizumab, ranibizumab, or a combination thereof) encapsulated in the silk matrix can provide a therapeutic effect for at least about 3 months, at least about 4 months, at least about 5 months, at least about 6 months, at least about 9 months, at least about 12 months, at least about 18 months, at least about 24 months or longer.

A kit provided herein can generally comprise at least one container containing one or more embodiments of the composition described herein, and/or at least one ocular delivery device in accordance with one or more embodiments described herein. In some embodiments, the composition described herein can be pre-loaded into at least one ocular delivery device described herein. For example, in one embodiment, a syringe can be pre-loaded with one or more embodiments of the composition described herein. In some embodiments, the pre-filled syringe can be further pre-attached to an injection needle. In other embodiments, the pre-filled syringe can be detached from the injection needle, which can be attached to the pre-filled syringe when in use. In some embodiments, e.g., where the composition is not provided or pre-loaded in the ocular delivery device, the kit can further comprise, e.g., a syringe and an injection needle for loading prior to use. In some embodiments, the kit can further comprise an anesthetic agent, e.g., an anesthetic agent that is commonly used during ocular administration. In some embodiments, the kit can further an antiseptic agent, e.g., to sterilize a target administration site. In some embodiments, the kit can further comprise one or more swabs to apply the antiseptic agent onto the target administration site, e.g., before, during and/or after the administration.

Without limitations, methods of sustained delivery described herein can be applicable for administering, to a subject or a target site (e.g., any tissue or organ, a wound, an infection site) of a subject, a pharmaceutically active agent (or a therapeutic agent) that requires relatively frequent administration. For example, a pharmaceutically active agent that requires administration at least once every three months, at least once every two months, at least once every week, at least once daily for a period of time, for example over a period of at least one week, at least two weeks, at least three weeks, at least four weeks, at least one month, at least two months, at least three months, at least four months, at least five months, at least six months, at least one years, at least two years or longer, can be dispersed or encapsulated in a silk matrix described herein for sustained-release formulations.

Embodiments of the Various Aspects Described Herein can be Illustrated by the Following Numbered Paragraphs:
1. A composition for ocular administration comprising a therapeutic agent encapsulated in a silk matrix, wherein an amount of the therapeutic agent encapsulated in the silk matrix provides a therapeutic effect for a period of time which is longer than when the same amount of the therapeutic agent is administered without the silk matrix.
2. The composition of paragraph 1, wherein the therapeutic effect comprises a therapeutic effect for treatment of an ocular condition.
3. The composition of paragraph 2, wherein the therapeutic effect for treatment of the ocular condition includes a reduction of at least one symptom associated with the ocular condition by at least about 10%.
4. The composition of any of paragraphs 1-3, wherein the period of time is at least about 1 week longer than when the same amount of the therapeutic agent is administered without the silk matrix.
5. The composition of any of paragraphs 1-4, wherein the period of time is at least about 1 month, at least about 3 months, or at least about 6 months longer than when the same amount of the therapeutic agent is administered without the silk matrix.
6. The composition of any of paragraphs 1-5, wherein the therapeutic agent is selected from the group consisting of proteins, peptides, antigens, immunogens, vaccines, antibodies or portions thereof, antibody-like molecules, enzymes, nucleic acids, siRNA, shRNA, aptamers, small molecules, antibiotics, and any combinations thereof.
7. The composition of any of paragraphs 1-6, wherein the therapeutic agent is an agent for treatment of an ocular condition.
8. The composition of any of paragraphs 1-7, wherein the therapeutic agent is selected from the group consisting of bevacizumab, ranibizumab, aflibercept, pegaptanib, tivozanib, fluocinolone acetonide, ganciclovir, triamcinolone acetonide, foscarnet, vancomycin, ceftazidime, amikacin, amphotericin B, dexamethasone, and any combinations thereof.
9. The composition of any of paragraphs 1-8, wherein the therapeutic agent comprises an angiogenesis inhibitor.
10. The composition of paragraph 9, wherein the angiogenesis inhibitor comprises a VEGF inhibitor.
11. The composition of paragraph 10, wherein the VEGF inhibitor is selected from the group consisting of bevacizumab, ranibizumab, aflibercept, pegaptanib, tivozanib, 3-(4-Bromo-2,6-difluoro-benzyloxy)-5-[3-(4-pyrrolidin 1-yl-butyl)-ureido]-isothiazole-4-carboxylic acid amide hydrochloride, axitinib, N-(4-bromo-2-fluorophenyl)-6-methoxy-7-[(1-methylpiperidin-4-yl)methoxy]quinazolin-4-amine, an inhibitor of VEGF-R2 and VEGF-R1, axitinib, N,2-dimethyl-6-(2-(1-methyl-1H-imidazol-2-yl)thieno[3,2-b]pyridin-7-yloxy)benzo[b]thiophene-3-carboxamide, tyrosine kinase inhibitor of the RET/PTC oncogenic kinase, N-(4-bromo-2-fluorophenyl)-6-methoxy-7-[(1-methylpiperidin-4-yl) methoxy]quinazolin-4-amine, pan-VEGF-R-kinase inhibitor; protein kinase inhibitor, multitargeted human epidermal receptor (HER) 1/2 and vascular endothelial growth factor receptor (VEGFR) 1/2 receptor family tyrosine kinases inhibitor, cediranib, sorafenib, vatalanib, glufanide disodium, VEGFR2-selective monoclonal antibody, angiozyme, an siRNA-based VEGFR1 inhibitor, Fumagillin and analogue thereof, soluble ectodomains of the VEGF receptors, shark cartilage and derivatives thereof, 5-((7-Benzyloxyquinazolin-4-yl)amino)-4-fluoro-2-methyl phenol hydrochloride, any derivatives thereof and any combinations thereof.
12. The composition of paragraph 10 or 11, wherein the VEGF inhibitor is bevacizumab, ranibizumab, or a combination thereof.
13. The composition of any of paragraphs 1-12, wherein the therapeutic agent or the VEGF inhibitor is present in an amount of about 0.01 mg to about 50 mg.
14. The composition of any of paragraphs 1-13, wherein the therapeutic agent or the VEGF inhibitor is present in an amount of about 1.5 mg to about 10 mg, or about 5 mg to about 10 mg.
15. The composition of any of paragraphs 1-14, wherein the silk matrix comprises silk fibroin at a concentration of about 0.1% (w/v) to about 50% (w/v).
16. The composition of any of paragraphs 1-15, wherein the silk matrix comprises silk fibroin at a concentration of about 0.5% (w/v) to about 30% (w/v).
17. The composition of any of paragraphs 1-16, wherein the silk matrix comprises silk fibroin at a concentration of about 1% (w/v) to about 15% (w/v).
18. The composition of any of paragraphs 1-17, wherein the silk matrix further comprises a biocompatible polymer.
19. The composition of paragraph 18, wherein the biocompatible polymer is selected from the group consisting of a poly-lactic acid (PLA), poly-glycolic acid (PGA), poly-lactide-co-glycolide (PLGA), polyesters, poly(ortho ester), poly(phosphazine), poly(phosphate ester), polycaprolactone, gelatin, collagen, cellulose, hyaluronan, poly(ethylene glycol) (PEG), triblock copolymers, polylysine and any derivatives thereof.
20. The composition of any of paragraphs 1-19, wherein the silk matrix is selected from the group consisting of hydrogel, microparticle, nanoparticle, fiber, film, lyophilized powder, lyophilized gel, reservoir implant, homogenous implant, tube, gel-like or gel particle, and any combinations thereof.
21. The composition of any of paragraphs 1-20, wherein the silk matrix comprises a hydrogel.
22. The composition of any of paragraphs 1-20, wherein the silk matrix comprises a microparticle, a nanoparticle, or a gel-like or gel particle.
23. The composition of paragraph 22, wherein the microparticle, the nanoparticle, or the gel-like or gel particle encapsulating the therapeutic agent is embedded in a solid substrate.
24. The composition of paragraph 23, wherein the solid substrate is selected from the group consisting of a tablet, a capsule, a microchip, a hydrogel, a mat, a film, a fiber, an ocular delivery device, an implant, a tube, a coating, and any combinations thereof.
25. The composition of paragraph 23 or 24, wherein the solid substrate comprises a hydrogel.
26. The composition of paragraph 25, wherein the hydrogel comprises a silk hydrogel.
27. The composition of any of paragraphs 1-26, wherein the composition is adapted to be injectable.
28. The composition of paragraph 27, wherein the composition is pre-loaded into a syringe.
29. The composition of paragraph 28, wherein the syringe is further attached to an injection needle.
30. The composition of any of paragraphs 1-29, wherein the ocular administration is administration of the composition to at least a portion of an eye selected from the group consisting of lens, sclera, conjunctiva, aqueous humor, ciliary muscle, and vitreous humor.
31. The composition of any of paragraphs 1-30, wherein the ocular administration is intravitreal administration.
32. An ocular delivery device comprising the composition of any of paragraphs 1-31.
33. The ocular delivery device of paragraph 32, wherein the composition is pre-loaded into the ocular delivery device.
34. The ocular delivery device of paragraph 32, wherein the device is a syringe with or without an injection needle.
35. The ocular delivery device of paragraph 34, wherein the injection needle is a 25- to 34-gauge needle.
36. The ocular delivery device of paragraph 34 or 35, wherein the injection needle is a 27- to 30-gauge needle.
37. The ocular delivery device of paragraph 32, wherein the device comprises a contact lens.
38. The ocular delivery device of paragraph 32, wherein the device comprises an eye-dropper.
39. The ocular delivery device of paragraph 32, wherein the device comprises a microneedle.
40. The ocular delivery device of paragraph 39, wherein the microneedle is a silk microneedle.
41. The ocular delivery device of paragraph 32, wherein the device is an implant.
42. A kit comprising a container containing a composition of any of paragraphs 1-31, or an ocular delivery device of any of paragraphs of 32-41.
43. The kit of paragraph 42, further comprising at least a syringe and an injection needle.
44. The kit of paragraph 43, wherein the injection needle is a 25- to 34-gauge needle.
45. The kit of paragraph 43 or 44, wherein the injection needle is a 27- to 30-gauge needle.
46. The kit of any of paragraphs 42-45, further comprising an anesthetic.
47. The kit of any of paragraphs 42-46, further comprising an antiseptic agent.
48. The kit of any of paragraphs 42-47, wherein the ocular delivery device is pre-loaded with the composition.
49. The kit of paragraph 48, wherein the ocular delivery device is a syringe with or without an injection needle.
50. A method for delivering a therapeutic agent to a target site of an eye comprising administering to a target site of an eye a therapeutic agent encapsulated in a silk matrix, wherein an amount of the therapeutic agent encapsulated in the silk matrix provides a therapeutic effect for a period of time which is longer than when the same amount of the therapeutic agent is administered without the silk matrix.
51. A method for treating an ocular condition in a subject comprising administering to target site of an eye of a subject a composition of any of paragraphs 1-29, thereby treating the ocular condition with a sustained release of the therapeutic agent to the target site of the eye.
52. The method of paragraph 51, wherein the ocular condition is a condition of a posterior segment of the eye.
53. The method of paragraph 51 or 52, wherein the ocular condition is selected from the group consisting of age-related macular degeneration, choroidal neovascularization, diabetic macular edema, acute and chronic macular neuroretinopathy, central serous chorioretinopathy, macular edema, acute multifocal placoid pigment epitheliopathy, Behcet's disease, birdshot retinochoroidopathy, posterior uveitis, posterior scleritis, serpignous choroiditis, subretinal fibrosis, uveitis syndrome, Vogt-Koyanagi-Harada syndrome, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, carotid artery disease (CAD), frosted branch angitis, sickle cell retinopathy, angioid streaks, familial exudative vitreoretinopathy, Eales disease, proliferative vitreal retinopathy, diabetic retinopathy, retinal disease associated with tumors, congenital hypertrophy of the retinal pigment epithelium (RPE), posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors, myopic retinal degeneration, acute retinal pigment epithelitis, glaucoma, endophthalmitis, cytomegalovirus retinitis, retinal cancers and any combinations thereof.
54. The method of paragraph 53, wherein the ocular condition is age-related macular degeneration.
55. The method of paragraph 53, wherein the therapeutic agent comprises a VEGF inhibitor.
56. The method of paragraph 55, wherein the VEGF inhibitor comprises bevacizumab, ranibizumab, or a combination thereof.
57. A method for administrating a therapeutic agent to a target site of an eye of a subject in need thereof comprising administrating to a target site of an eye of a subject the composition of any of paragraphs 1-31 at an administration frequency less than when the same amount of the therapeutic agent is administered without the silk matrix.
58. The method of paragraph 57, wherein the administration frequency is reduced by a factor of ½.
59. A method for increasing an effective amount of a therapeutic agent administered to an eye comprising administering to a target site of an eye a therapeutic agent encapsulated in a silk matrix, wherein upon administration, leakage of the therapeutic agent from the target site is reduced, as compared to when the same amount of the therapeutic agent is administered without the silk matrix, thereby increasing the effective amount of the therapeutic agent administered to the target site of the eye.
60. The method of any of paragraphs 50-59, wherein the therapeutic agent encapsulated in the silk matrix or the composition is administered to the anterior segment of the eye.
61. The method of any of paragraphs 50-60, wherein the therapeutic agent encapsulated in the silk matrix or the composition is administered to the posterior segment of the eye.
62. The method of any of paragraphs 50-61, wherein the therapeutic agent encapsulated in the silk matrix or the composition is administered to at least a portion of the eye selected from the group consisting of lens, sclera, conjunctiva, aqueous humor, ciliary muscle, and vitreous humor.
63. The method of any of paragraphs 50-62, wherein the therapeutic agent encapsulated in the silk matrix or the composition is administered to the vitreous humor of the eye.
64. The method of any of paragraphs 50-63, wherein the therapeutic agent encapsulated in the silk matrix or the composition is administered to the eye by injection.
65. The method of paragraph 64, wherein the injection is performed with a needle with a gauge of about 25 to about 34.
66. Then method paragraph 64 or 65, wherein the injection is performed with a needle with a gauge of about 27 to about 30.
67. The method of any of paragraphs 50-66, wherein the administration is performed no more than once a month.
68. The method of any of paragraphs 50-67, wherein the administration is performed no more than once every two months.
69. The method of any of paragraphs 50-68, wherein the administration is performed no more than once every three months, no more than once every four months or no more once every six months.
70. The method of any of paragraphs 50-69, wherein the therapeutic agent is selected from the group consisting of proteins, peptides, antigens, immunogens, vaccines, antibodies or portions thereof, antibody-like molecules, enzymes, nucleic acids, siRNA, shRNA, aptamers, small molecules, antibiotics, and any combinations thereof.
71. The method of any of paragraphs 50-70, wherein the therapeutic agent is selected from the group consisting of bevacizumab, ranibizumab, aflibercept, pegaptanib, tivozanib, fluocinolone acetonide, ganciclovir, triamcinolone acetonide, foscarnet, vancomycin, ceftazidime, amikacin, amphotericin B, dexamethasone, and any combinations thereof.
72. The method of any of paragraphs 50-71, wherein the therapeutic agent comprises an angiogenesis inhibitor.
73. The method of any of paragraphs 50-72, wherein the angiogenesis inhibitor comprises a VEGF inhibitor.
74. The method of paragraph 73, wherein the VEGF inhibitor is selected from the group consisting of bevacizumab, ranibizumab, aflibercept, pegaptanib, tivozanib, 3-(4-Bromo-2,6-difluoro-benzyloxy)-5-[3-(4-pyrrolidin 1-yl-butyl)-ureido]-isothiazole-4-carboxylic acid amide hydrochloride, axitinib, N-(4-bromo-2-fluorophenyl)-6-methoxy-7-[(1-methylpiperidin-4-yl)methoxy]quinazolin-4-amine, an inhibitor of VEGF-R2 and VEGF-R1, axitinib, N,2-dimethyl-6-(2-(1-methyl-1H-imidazol-2-yl)thieno[3,2-b]pyridin-7-yloxy)benzo[b]thiophene-3-carboxamide, tyrosine kinase inhibitor of the RET/PTC oncogenic kinase, N-(4-bromo-2-fluorophenyl)-6-methoxy-7-[(1-methylpiperidin-4-yl) methoxy]quinazolin-4-amine, pan-VEGF-R-kinase inhibitor; protein kinase inhibitor, multitargeted human epidermal receptor (HER) 1/2 and vascular endothelial growth factor receptor (VEGFR) 1/2 receptor family tyrosine kinases inhibitor, cediranib, sorafenib, vatalanib, glufanide disodium, VEGFR2-selective monoclonal antibody, angiozyme, an siRNA-based VEGFR1 inhibitor, Fumagillin and analogue thereof, soluble ectodomains of the VEGF receptors, shark cartilage and derivatives thereof, 5-((7-Benzyloxyquinazolin-4-yl)amino)-4-fluoro-2-methyl phenol hydrochloride, any derivatives thereof and any combinations thereof.
75. The method of paragraph 74, wherein the VEGF inhibitor comprises bevacizumab, ranibizumab, or a combination thereof.
76. The method of any of paragraphs 50-75, wherein the therapeutic agent or the VEGF inhibitor is present in an amount of about 0.01 mg to about 50 mg.
77. The method of any of paragraphs 50-76, wherein the therapeutic agent or the VEGF inhibitor is present in an amount of about 1.5 mg to about 10 mg, or about 5 mg to about 10 mg.
78. The method of any of paragraphs 50-77, wherein the silk matrix comprises silk fibroin at a concentration of about 0.1% (w/v) to about 50% (w/v).
79. The method of any of paragraphs 50-78, wherein the silk matrix comprises silk fibroin at a concentration of about 0.5% (w/v) to about 30% (w/v).
80. The method of any of paragraphs 50-79, wherein the silk matrix comprises silk fibroin at a concentration of about 1% (w/v) to about 15% (w/v).
81. The method of any of paragraphs 50-80, wherein the silk matrix further comprises a biocompatible polymer.
82. The method of paragraph 81, wherein the biocompatible polymer is selected from the group consisting of a poly-lactic acid (PLA), poly-glycolic acid (PGA), poly-lactide-co-glycolide (PLGA), polyesters, poly(ortho ester), poly(phosphazine), poly(phosphate ester), polycaprolactone, gelatin, collagen, cellulose, hyaluronan, poly(ethylene glycol) (PEG), triblock copolymers, polylysine and any derivatives thereof.
83. The method of any of paragraphs 50-82, wherein the silk matrix is selected from the group consisting of hydrogel, microparticle, nanoparticle, fiber, film, lyophilized powder, lyophilized gel, reservoir implant, homogenous implant, a tube, gel-like or gel particle, and any combinations thereof.
84. The method of any of paragraphs 50-83, wherein the silk matrix comprises a hydrogel.
85. The method of any of paragraphs 50-84, wherein the silk matrix comprises a microparticle, a nanoparticle, or a gel-like or gel particle.
86. The method of paragraph 85, wherein the microparticle, the nanoparticle, or the gel-like or gel particle encapsulating the therapeutic agent is embedded in a solid substrate.
87. The method of paragraph 86, wherein the solid substrate is selected from the group consisting of a tablet, a capsule, a microchip, a hydrogel, a mat, a film, a fiber, an ocular delivery device, an implant, a tube, a coating, and any combinations thereof.
88. The method of paragraph 86 or 87, wherein the solid substrate comprises a hydrogel.
89. The method of paragraph 88, wherein the hydrogel comprises a silk hydrogel.
90. The method of any of paragraphs 50-89, wherein the therapeutic agent is released from the silk matrix at a rate such that at least about 20% of the therapeutic agent initially encapsulated in the silk matrix is released over a period of at least about 3 months.
91. The method of any of paragraphs 50-90, wherein the therapeutic agent is released from the silk matrix at the rate such that at least about 40% of the therapeutic agent initially encapsulated in the silk matrix is released over a period of at least about 3 months.
92. The method of any of paragraphs 50-91, wherein the therapeutic agent is released from the silk matrix at the rate such that at least about 60% of the therapeutic agent initially encapsulated in the silk matrix is released over a period of at least about 3 months.
93. The method of any of paragraphs 50-92, wherein the therapeutic agent is released from the silk matrix at the rate of about 1 ng/day to about 15 mg/day.
94. The method of any of paragraphs 50-93, wherein the therapeutic agent is released from the silk matrix at the rate of about 1 μg/day to about 1 mg/day.
95. The method of any of paragraphs 50-94, wherein the therapeutic effect provided by the amount of the therapeutic agent encapsulated in the silk matrix comprises a therapeutic effect for treatment of an ocular condition.
96. The method of paragraph 95, wherein the therapeutic effect for treatment of the ocular condition comprises a reduction of at least one symptom associated with the ocular condition by at least about 10%.
97. The method of any of paragraphs 50-96, wherein the period of time of the therapeutic effect provided by the amount of the therapeutic agent encapsulated in the silk matrix is at least about 1 week longer than when the same amount of the therapeutic agent is administered without the silk matrix.
98. The method of any of paragraphs 50-97, wherein the period of time of the therapeutic effect provided by the amount of the therapeutic agent encapsulated in the silk matrix is at least about 1 month longer than when the same amount of the therapeutic agent is administered without the silk matrix.
99. The method of any of paragraphs 50-98, wherein the period of time of the therapeutic effect provided by the amount of the therapeutic agent encapsulated in the silk matrix is at least about 3 months longer than when the same amount of the therapeutic agent is administered without the silk matrix.
100. The method of any of paragraphs 50-99, wherein the period of time of the therapeutic effect provided by the amount of the therapeutic agent encapsulated in the silk matrix is at least about 6 months longer than when the same amount of the therapeutic agent is administered without the silk matrix.
101. A method of producing a controlled-release silk-based composition for ocular administration comprising contacting with water vapor a silk-based matrix, the silk matrix comprising at least one therapeutic agent encapsulated therein.
102. The method of paragraph 101, wherein the silk-based matrix to be contacted with the water vapor is a non-crosslinked silk-based matrix.
103. The method of paragraph 101 or 102, wherein the contact of the silk-based matrix with water vapor induces formation of beta sheet structures in silk fibroin.
104. The method of any of paragraphs 101-103, wherein the contact of the silk-based matrix with the water vapor modulates release kinetics of said at least one therapeutic agent from the silk-based matrix.
105. The method of any of paragraphs 101-104, wherein said at least one therapeutic agent is selected from the group consisting of proteins, peptides, antigens, immunogens, vaccines, antibodies or portions thereof, antibody-like molecules, enzymes, nucleic acids, siRNA, shRNA, aptamers, small molecules, antibiotics, and any combinations thereof.
106. The method of any of paragraphs 101-105, wherein the therapeutic agent is selected from the group consisting of bevacizumab, ranibizumab, aflibercept, pegaptanib, tivozanib, fluocinolone acetonide, ganciclovir, triamcinolone acetonide, foscarnet, vancomycin, ceftazidime, amikacin, amphotericin B, dexamethasone, and any combinations thereof.
107. The method of any of paragraphs 101-106, wherein the therapeutic agent comprises an angiogenesis inhibitor.
108. The method of paragraph 107, wherein the angiogenesis inhibitor comprises a VEGF inhibitor.
109. The method of paragraph 108, wherein the VEGF inhibitor is selected from the group consisting of bevacizumab, ranibizumab, aflibercept, pegaptanib, tivozanib, 3-(4-Bromo-2,6-difluoro-benzyloxy)-5-[3-(4-pyrrolidin 1-yl-butyl)-ureido]-isothiazole-4-carboxylic acid amide hydrochloride, axitinib, N-(4-bromo-2-fluorophenyl)-6-methoxy-7-[(1-methylpiperidin-4-yl)methoxy]quinazolin-4-amine, an inhibitor of VEGF-R2 and VEGF-R1, axitinib, N,2-dimethyl-6-(2-(1-methyl-1H-imidazol-2-yl)thieno[3,2-b]pyridin-7-yloxy)benzo[b]thiophene-3-carboxamide, tyrosine kinase inhibitor of the RET/PTC oncogenic kinase, N-(4-bromo-2-fluorophenyl)-6-methoxy-7-[(1-methylpiperidin-4-yl) methoxy]quinazolin-4-amine, pan-VEGF-R-kinase inhibitor; protein kinase inhibitor, multitargeted human epidermal receptor (HER) 1/2 and vascular endothelial growth factor receptor (VEGFR) 1/2 receptor family tyrosine kinases inhibitor, cediranib, sorafenib, vatalanib, glufanide disodium, VEGFR2-selective monoclonal antibody, angiozyme, an siRNA-based VEGFR1 inhibitor, Fumagillin and analogue thereof, soluble ectodomains of the VEGF receptors, shark cartilage and derivatives thereof, 5-((7-Benzyloxyquinazolin-4-yl)amino)-4-fluoro-2-methyl phenol hydrochloride, any derivatives thereof and any combinations thereof.
110. The method of paragraph 108, wherein the VEGF inhibitor comprises bevacizumab, ranibizumab, or a combination thereof.
111. The method of any of paragraphs 101-110, wherein the silk matrix is selected from the group consisting of hydrogel, microparticle, nanoparticle, fiber, film, lyophilized powder, lyophilized gel, reservoir implant, homogenous implant, a tube, gel-like or gel particle, and any combinations thereof.
112. The method of any of paragraphs 101-111, wherein said at least one therapeutic agent is encapsulated in a silk hydrogel.
113. The method of any of paragraphs 101-112, wherein said at least one therapeutic agent is encapsulated in silk microparticles, silk nanoparticles, gel-like or gel particles, or any combinations thereof.
114. The method of paragraph 113, the silk microparticles, silk nanoparticles, gel-like or gel particles encapsulating said at least one therapeutic agent are further embedded in a hydrogel.
115. The method of paragraph 114, wherein the hydrogel comprises a silk hydrogel.

### Some Selected Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed inventions, because the scope of the inventions is limited only by the claims. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean±5% of the value being referred to. For example, about 100 means from 95 to 105.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, the terms “proteins” and “peptides” are used interchangeably herein to designate a series of amino acid residues connected to the other by peptide bonds between the alpha-amino and carboxy groups of adjacent residues. The terms “protein”, and “peptide”, which are used interchangeably herein, refer to a polymer of protein amino acids, including modified amino acids (e.g., phosphorylated, glycated, etc.) and amino acid analogs, regardless of its size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins and fragments of proteins, unless otherwise noted. The terms “protein” and “peptide” are used interchangeably herein when referring to a gene product and fragments thereof. Thus, exemplary peptides or proteins include gene products, naturally occurring proteins, homologs, orthologs, paralogs, fragments and other equivalents, variants, fragments, and analogs of the foregoing.

The term “nucleic acids” used herein refers to polynucleotides such as deoxyribonucleic acid (DNA), and, where appropriate, ribonucleic acid (RNA), polymers thereof in either single- or double-stranded form. Unless specifically limited, the term encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985), and Rossolini, et al., Mol. Cell. Probes 8:91-98 (1994)). The term “nucleic acid” should also be understood to include, as equivalents, derivatives, variants and analogs of either RNA or DNA made from nucleotide analogs, and, single (sense or antisense) and double-stranded polynucleotides.

The term “short interfering RNA” (siRNA), also referred to herein as “small interfering RNA” is defined as an agent which functions to inhibit expression of a target gene, e.g., by RNAi. An siRNA can be chemically synthesized, it can be produced by in vitro transcription, or it can be produced within a host cell. siRNA molecules can also be generated by cleavage of double stranded RNA, where one strand is identical to the message to be inactivated. The term “siRNA” refers to small inhibitory RNA duplexes that induce the RNA interference (RNAi) pathway. These molecules can vary in length (generally 18-30 base pairs) and contain varying degrees of complementarity to their target mRNA in the antisense strand. Some, but not all, siRNA have unpaired overhanging bases on the 5′ or 3′ end of the sense 60 strand and/or the antisense strand. The term “siRNA” includes duplexes of two separate strands, as well as single strands that can form hairpin structures comprising a duplex region.

The term “shRNA” as used herein refers to short hairpin RNA which functions as RNAi and/or siRNA species but differs in that shRNA species are double stranded hairpin-like structure for increased stability. The term “RNAi” as used herein refers to interfering RNA, or RNA interference molecules are nucleic acid molecules or analogues thereof for example RNA-based molecules that inhibit gene expression. RNAi refers to a means of selective post-transcriptional gene silencing. RNAi can result in the destruction of specific mRNA, or prevents the processing or translation of RNA, such as mRNA.

The term “enzymes” as used here refers to a protein molecule that catalyzes chemical reactions of other substances without it being destroyed or substantially altered upon completion of the reactions. The term can include naturally occurring enzymes and bioengineered enzymes or mixtures thereof. Examples of enzyme families include kinases, dehydrogenases, oxidoreductases, GTPases, carboxyl transferases, acyl transferases, decarboxylases, transaminases, racemases, methyl transferases, formyl transferases, and α-ketodecarboxylases.

The term “vaccines” as used herein refers to any preparation of killed microorganisms, live attenuated organisms, subunit antigens, toxoid antigens, conjugate antigens or other type of antigenic molecule that when introduced into a subjects body produces immunity to a specific disease by causing the activation of the immune system, antibody formation, and/or creating of a T-cell and/or B-cell response. Generally vaccines against microorganisms are directed toward at least part of a virus, bacteria, parasite, mycoplasma, or other infectious agent.

As used herein, the term “aptamers” means a single-stranded, partially single-stranded, partially double-stranded or double-stranded nucleotide sequence capable of specifically recognizing a selected non-oligonucleotide molecule or group of molecules. In some embodiments, the aptamer recognizes the non-oligonucleotide molecule or group of molecules by a mechanism other than Watson-Crick base pairing or triplex formation. Aptamers can include, without limitation, defined sequence segments and sequences comprising nucleotides, ribonucleotides, deoxyribonucleotides, nucleotide analogs, modified nucleotides and nucleotides comprising backbone modifications, branchpoints and nonnucleotide residues, groups or bridges. Methods for selecting aptamers for binding to a molecule are widely known in the art and easily accessible to one of ordinary skill in the art.

As used herein, the term “antibody” or “antibodies” refers to an intact immunoglobulin or to a monoclonal or polyclonal antigen-binding fragment with the Fc (crystallizable fragment) region or FcRn binding fragment of the Fc region. The term “antibodies” also includes “antibody-like molecules”, such as fragments of the antibodies, e.g., antigen-binding fragments. Antigen-binding fragments can be produced by recombinant DNA techniques or by enzymatic or chemical cleavage of intact antibodies. “Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies, and polypeptides that contain at least a portion of an immunoglobulin that is sufficient to confer specific antigen binding to the polypeptide. Linear antibodies are also included for the purposes described herein. The terms Fab, Fc, pFc′, F(ab′) 2 and Fv are employed with standard immunological meanings (Klein, Immunology (John Wiley, New York, N.Y., 1982); Clark, W. R. (1986) The Experimental Foundations of Modern Immunology (Wiley & Sons, Inc., New York); and Roitt, I. (1991) Essential Immunology, 7th Ed., (Blackwell Scientific Publications, Oxford)). Antibodies or antigen-binding fragments specific for various antigens are available commercially from vendors such as R&D Systems, BD Biosciences, e-Biosciences and Miltenyi, or can be raised against these cell-surface markers by methods known to those skilled in the art.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop.

The expression “linear antibodies” refers to the antibodies described in Zapata et al., Protein Eng., 8(10):1057-1062 (1995). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

The expression “single-chain Fv” or “scFv” antibody fragments, as used herein, is intended to mean antibody fragments that comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. (The Pharmacology of Monoclonal Antibodies, vol. 113, Rosenburg and Moore eds., Springer-Verlag, New York, pp. 269-315 (1994)).

The term “diabodies,” as used herein, refers to small antibody fragments with two antigen-binding sites, which fragments comprise a heavy-chain variable domain (VH) Connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. (EP 404,097; WO 93/11161; Hollinger et ah, Proc. Natl. Acad. Sd. USA, P0:6444-6448 (1993)).

As used herein, the term “small molecules” refers to natural or synthetic molecules including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “antibiotics” is used herein to describe a compound or composition which decreases the viability of a microorganism, or which inhibits the growth or reproduction of a microorganism. As used in this disclosure, an antibiotic is further intended to include an antimicrobial, bacteriostatic, or bactericidal agent. Exemplary antibiotics include, but are not limited to, penicillins, cephalosporins, penems, carbapenems, monobactams, aminoglycosides, sulfonamides, macrolides, tetracyclines, lincosides, quinolones, chloramphenicol, vancomycin, metronidazole, rifampin, isoniazid, spectinomycin, trimethoprim, sulfamethoxazole, and the like.

As used herein, the term “antigens” refers to a molecule or a portion of a molecule capable of being bound by a selective binding agent, such as an antibody, and additionally capable of being used in an animal to elicit the production of antibodies capable of binding to an epitope of that antigen. An antigen may have one or more epitopes. The term “antigen” can also refer to a molecule capable of being bound by an antibody or a T cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens.

The term “immunogen” refers to any substance, e.g., vaccines, capable of eliciting an immune response in an organism. An “immunogen” is capable of inducing an immunological response against itself on administration to a subject. The term “immunological” as used herein with respect to an immunological response, refers to the development of a humoral (antibody mediated) and/or a cellular (mediated by antigen-specific T cells or their secretion products) response directed against an immunogen in a recipient subject. Such a response can be an active response induced by administration of an immunogen or immunogenic peptide to a subject or a passive response induced by administration of antibody or primed T-cells that are directed towards the immunogen. A cellular immune response is elicited by the presentation of polypeptide epitopes in association with Class I or Class II MHC molecules to activate antigen-specific CD4+ T helper cells and/or CD8+ cytotoxic T cells. Such a response can also involve activation of monocytes, macrophages, NK cells, basophils, dendritic cells, astrocytes, microglia cells, eosinophils or other components of innate immunity.

The terms “decrease”, “reduced”, “reduction”, “decrease” are all used herein generally to mean a decrease by a statistically significant amount. However, for avoidance of doubt, “reduced”, “reduction” or “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased” and “increase” are all used herein to generally mean an increase by a statically significant amount; for the avoidance of any doubt, the terms “increased”, and “increase” mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means at least two standard deviation (2SD) away from a reference level. The term refers to statistical evidence that there is a difference. It is defined as the probability of making a decision to reject the null hypothesis when the null hypothesis is actually true.

As used interchangeably herein, the terms “essentially” and “substantially” means a proportion of at least about 60%, or preferably at least about 70% or at least about 80%, or at least about 90%, at least about 95%, at least about 97% or at least about 99% or more, or any integer between 70% and 100%. In some embodiments, the term “essentially” means a proportion of at least about 90%, at least about 95%, at least about 98%, at least about 99% or more, or any integer between 90% and 100%. In some embodiments, the term “essentially” can include 100%.

Although preferred embodiments have been depicted and described in detail herein, it will be apparent to those skilled in the relevant art that various modifications, additions, substitutions, and the like can be made without departing from the spirit of the inventions and these are therefore considered to be within the scope of the inventions as defined in the claims which follow. Further, to the extent not already indicated, it will be understood by those of ordinary skill in the art that any one of the various embodiments herein described and illustrated may be further modified to incorporate features shown in any of the other embodiments disclosed herein.

The disclosure is further illustrated by the following examples which should not be construed as limiting. The examples are illustrative only, and are not intended to limit, in any manner, any of the aspects described herein. The following examples do not in any way limit the inventions.

### EXAMPLES

### Examples of Materials and Methods Used in the Following Examples 1-3

Preparation of Sterile, Low-Endotoxin Aqueous Silk Fibroin Solution.

Using aseptic technique, aqueous silk fibroin solutions (6-8% (w/v)) were prepared from degummed silk fibers (purchased from Suhao Biomaterials Co. (Suzhou, China)). Briefly, degummed silk fibers were soaked in 70% ethanol in endotoxin-free glassware and sonicated for six hours with the ethanol solution being replaced every two hours. After drying in a laminar flow hood overnight, the silk fibers were dissolved in 9.3 M lithium bromide and dialyzed against deionized water for 48 hours. The resultant silk solutions were concentrated, if necessary, by dialysis against poly(ethylene glycol) (PEG) to produce 20-30% (w/v) silk fibroin solutions. All silk fibroin solutions were stored at 4° C. until for use to make hydrogel formulations.

Preparation of Concentrated Bevacizumab.

Using aseptic technique, 25 mg/mL (2.5% (w/v)) bevacizumab (available under the tradename AVASTIN® from Genentech) was concentrated up to 20-25% (w/v) bevacizumab. Briefly, AMICON® Ultra-15 centrifugal filter units (10,000 MWCO, Millipore) were sterilized by adding 70% ethanol to the filter, spinning down partially to sterilize the filter followed by incubation overnight. Excess ethanol was removed from the top of the filter with a pipette before adding pyrogen-free water to rinse. The water was spun down to rinse the filter, repeating 5 times with fresh water to remove all trace ethanol. AVASTIN® solution (2.5%) was then added to the top of the sterile filters and centrifuged until achieving the desired bevacizumab concentration (20% to 25% (w/v)).

Preparation of Bevacizumab-Loaded Silk Hydrogel Formulations.

Bevacizumab-loaded silk hydrogel formulations were prepared by mixing silk (0.5 to 25% (w/v)) and bevacizumab (2.5 to 25% (w/v)) solutions to achieve the desired final concentrations of silk and bevacizumab in the hydrogel formulation. For example, “low dose” hydrogels were prepared by mixing equal volumes of sterile 4% silk and concentrated bevacizumab (5%) to achieve final concentrations of 2% and 2.5%, respectively. Similarly, “high dose” hydrogels were prepared by mixing equal volumes of sterile 4% silk and concentrated bevacizumab (20%) to achieve final concentrations of 2% and 10%, respectively. To induce gelation, the mixed solutions were sonicated using a digital sonifier (Branson) under aseptic conditions. Following sonication, the resultant solutions were prepared for injection by drawing into a 1 mL syringe using a 16G-18G needle, withdrawing air from the syringe, and replacing the needle with a 27G-30G needle suitable for injection. The syringes were incubated overnight at 37° C. before switching to 4° C. for storage before injection. Samples of each formulation were tested to be sterile and endotoxin-free according to USP <71> and USP <85> guidelines, respectively.

Ocular Pharmacokinetic Evaluation of Bevacizumab-Loaded Silk Hydrogel Formulations.

Pharmacokinetic properties of bevacizumab-loaded silk hydrogels were evaluated over a 90 day period following intravitreal injection in Dutch belted rabbits. Levels of bevacizumab in the blood, aqueous humor, and vitreous humor were evaluated over at different time points over the 90-day period. Parallel in vitro studies were performed to determine release kinetics in phosphate buffered saline (PBS) solution over 90 days. Briefly, bevacizumab-loaded silk hydrogels were injected (50 μL/injection) into 4 mL of PBS with 0.02% (w/v) sodium azide, with release medium sampled (3.6 mL/sample) and replaced approximately every 3-4 days.

Analysis of Bevacizumab Levels.

An enzyme-linked immunosorbent assay (ELISA) was employed for determining bevacizumab concentration in in vitro and in vivo samples, as described in Hsei et al., Pharmaceutical Research,19:1753 (2002), with the modification of using recombinant human vascular endothelial growth factor 165 (VEGF165) as opposed to a truncated recombinant human VEGF as described. Briefly, VEGF165 was used as the capture peptide for bevacizumab, with a goat antibody to human IgG conjugated to horseradish peroxidase for detection in a sandwich assay. The detection limit of this ELISA assay was approximately 1 ng/mL for plasma, aqueous humor and vitreous humor and approximately 0.1 ng/mL for in vitro PBS samples. For early in vitro time points, high concentrations of bevacizumab in the release medium could render the ELISA assay inapplicable due to its relatively low detection limit. Accordingly, a gel permeation chromatography (GPC) method was employed using PBS as the solvent and an Agilent Bio SEC-3 column (4.6 mm×300 mm, 3 μm particle size, 300 Å pore size). A solvent flow rate of 0.4 mL/min to 0.5 mL/min was used and the injection volume was 5 μL/sample. Sample detection was via a multiwavelength detector at 280 nm. The detection limit of the GPC method was approximately 1 μg/mL (retention time≈8.0 min for a flow rate of 0.4 mL/min).

### Example 1

### In Vivo Pharmacokinetic Evaluation of Bevacizumab-Loaded Silk Hydrogel Formulations

In vivo studies entailed intravitreal injection of bevacizumab-loaded silk hydrogels in Dutch belted rabbits followed by sampling of blood, aqueous humor and vitreous humor as well as fundus photos taken at terminal sacrifice over a 90 day period. In brief, rabbits (n=9 per treatment) were injected into one eye (right) with 50 μL of one of the four test formulations: (i) negative vehicle control (i.e., silk hydrogel (2% silk) without therapeutic agent), (ii) positive control (i.e., 2.5% bevacizumab solution (1.25 mg bevacizumab)), (iii) “low dose” silk hydrogel (i.e., 2.5% (w/v) bevacizumab in 2% silk hydrogel (1.25 mg bevacizumab)), and (iv) “high dose” silk hydrogel (i.e., 10% (w/v) bevacizumab in 2% silk hydrogel (5 mg bevacizumab)). While one eye (right) was used for evaluation of the test formulation, the other eye (left) was used a control (i.e., without any injection). The positive control is based on the currently-employed dosage regimen of one injection of 50 μL of 2.5% bevacizumab solution (1.25 mg bevacizumab (AVASTIN®, Genentech)) per month. Ophthalmic examinations were performed periodically to assess the overall health of the rabbits' eyes as well as to monitor any degradation of hydrogel over time.

An exemplary dosing design for the 90-day ocular evaluation of bevacizumab-loaded silk hydrogels in Dutch belted rabbits is provided in Table 1.

TABLE 1
Dosing Design

| | | # of | | | | |
|---|---|---|---|---|---|---|
| | # of | Eyes | | Dose | | Dose |
| Group | Animals | Injected | Compound | (mg/eye) | | Volume |
| 1 | 9 | 1 | Negative vehicle | 0 | mg/eye | 50 μL |
| | | | control | | | per eye |
| 2 | | | Positive control | 1.25 | mg/eye | |
| 3 | | | Low dose gel | 1.25 | mg/eye | |
| 4 | | | High dose gel | 5.0 | mg/eye | |

Upon injection of the test formulations, it was found that there was less leakage from the injection site with the silk hydrogel formulations as compared to the positive solution control. Thus, the silk hydrogel formulations, in some embodiments, can act as a plug, providing more consistent and higher effective dosing as compared to the solution injections.

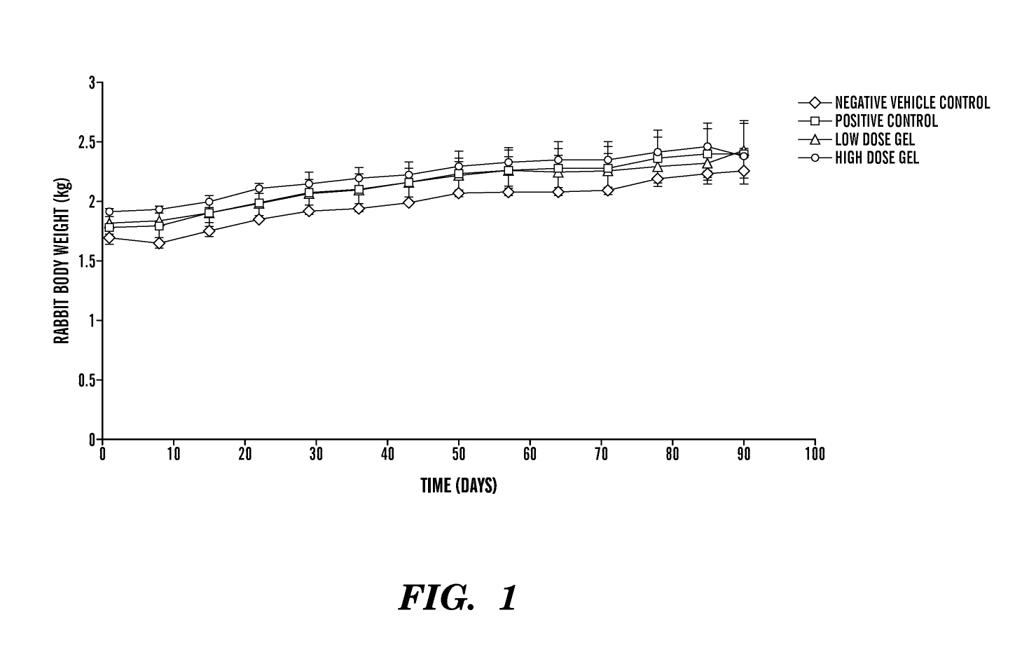

Rabbit body weights were monitored weekly over the 90-day period. As shown in FIG. 1, the rabbits continued to gain weight over the course of the 90-day period, indicating that there were no gross adverse reactions to the procedure or to the silk hydrogel formulations.

Pharmacokinetics of bevacizumab was determined based on plasma, aqueous humor and vitreous humor collected from rabbits over the 90 day period following injection. In particular, blood was collected from the rabbits before dosage and at Days 2, 4, 8 and weekly thereafter up to Day 90 post-dose, to obtain plasma for analysis. Additionally, dependent on overall health of the eye, aqueous humor was collected from rabbits at Days 8, 30, 59 and 90 days post-dose. Additionally, vitreous humor was collected from rabbits at sacrifice on Days 8, 30 and 90 days post-dose (n=3 rabbits per treatment sacrificed at each time point).

An exemplary sampling schedule for the 90-day ocular evaluation of bevacizumab-loaded silk hydrogels in Dutch belted rabbits is provided in Table 2.

TABLE 2
Pharmacokinetic Plasma and Aqueous Collection

| | | | | | Number |
|---|---|---|---|---|---|
| | | | Number | Number | of Ani- |
| | Blood | Aqueous | of Ani- | of Ani- | mals >Day |
| | Time | Time | mals | mals | 30 to |
| Group | Points | Points* | Day 1-8 | Day 30 | 90 Days |
| 1 | Pre-dose, Days | Days 2, 8, 30, | 9 | 6 | 3 |
| 2 | 2, 4, and 8, | 60 and up to | 9 | 6 | 3 |
| 3 | and weekly up | 90 days | 9 | 6 | 3 |
| 4 | to 90 days | post dose | 9 | 6 | 3 |
| | post dose | | | | |

*Frequency of collection will depend on the overall health of the eye. A terminal sample will be collected from each animal.

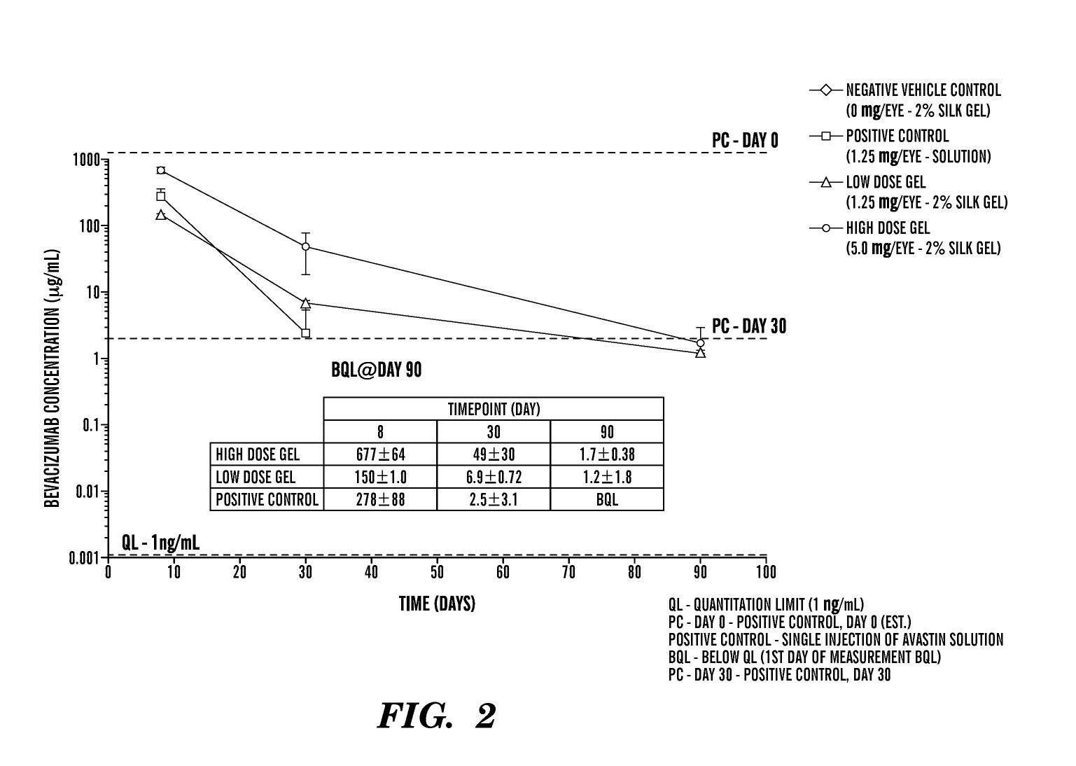

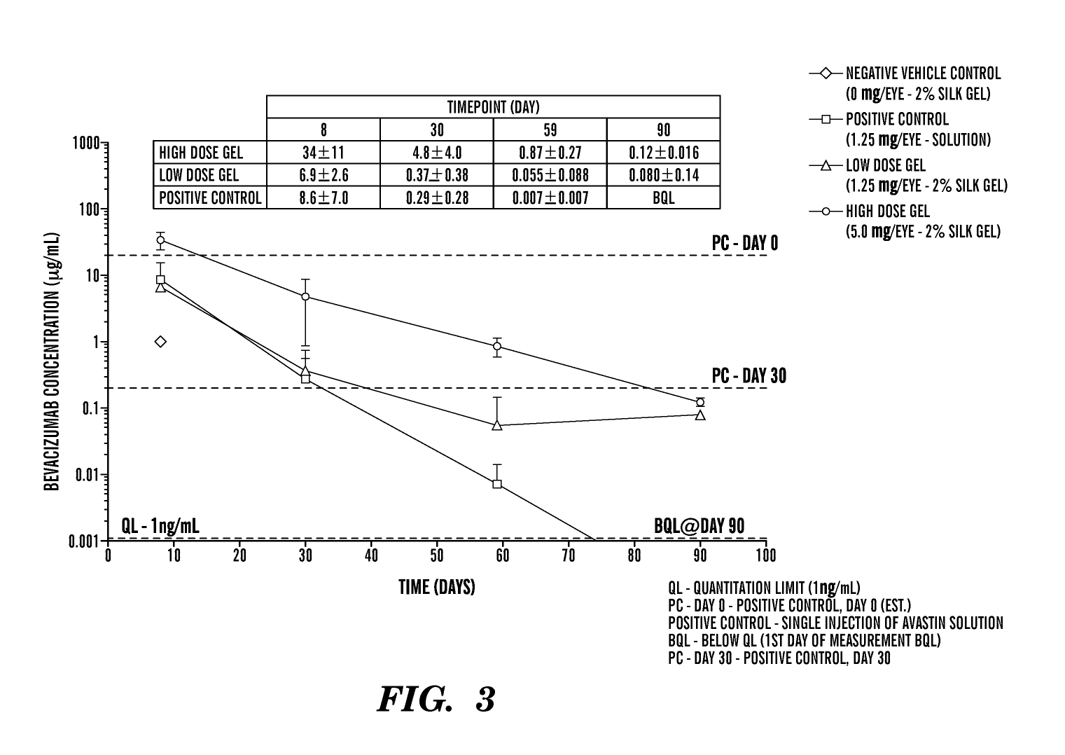

The samples collected from plasma, aqueous humor and vitreous humor were analyzed using the ELISA method. For both vitreous humor and aqueous humor, the concentration of bevacizumab at the 90 day time point for both “low dose” silk hydrogel and “high dose” silk hydrogel formulation was approximately equivalent to the concentration of bevacizumab present in the positive control after merely 30 days (i.e., about 1-3 μg/mL in the vitreous humor; see FIGS. 2 and 3, respectively). Moreover, the concentration of bevacizumab for the positive control was below the level of quantification at day 90. Accordingly, “low dose” and “high dose” silk hydrogel formulations provided therapeutic levels of bevacizumab for at least three times longer than the positive control.

Comparing the different sampling sites within different test formulations, the ratio between the vitreous and aqueous humor concentrations was approximately 20-fold, and thus can be used as a predictive measure, e.g., for determining the concentration of a therapeutic agent delivered to the vitreous humor by measuring the corresponding concentration in the aqueous humor instead.

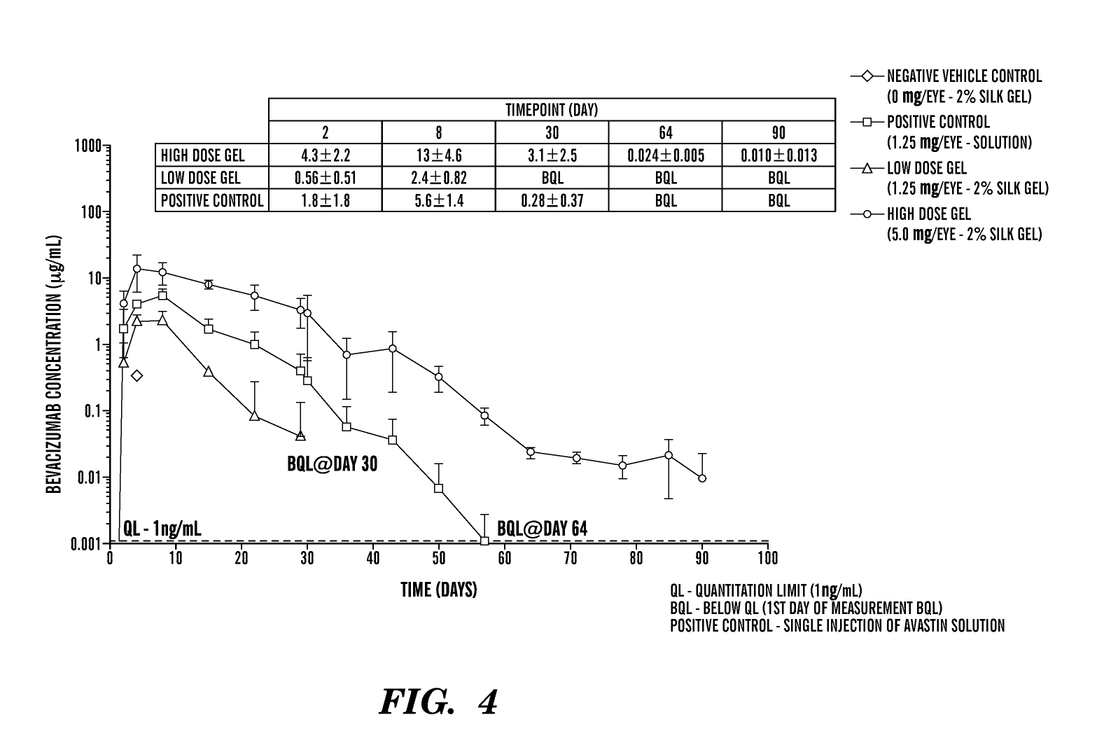

Unlike vitreous and aqueous humor, the concentration of bevacizumab in plasma of the “low dose” hydrogel formulation was below the quantification level after 30 days (see FIG. 4 as compared to FIGS. 2 and 3). This finding indicates that, relative to positive control, some embodiments of the hydrogel formulations can limit systemic exposure of bevacizumab.

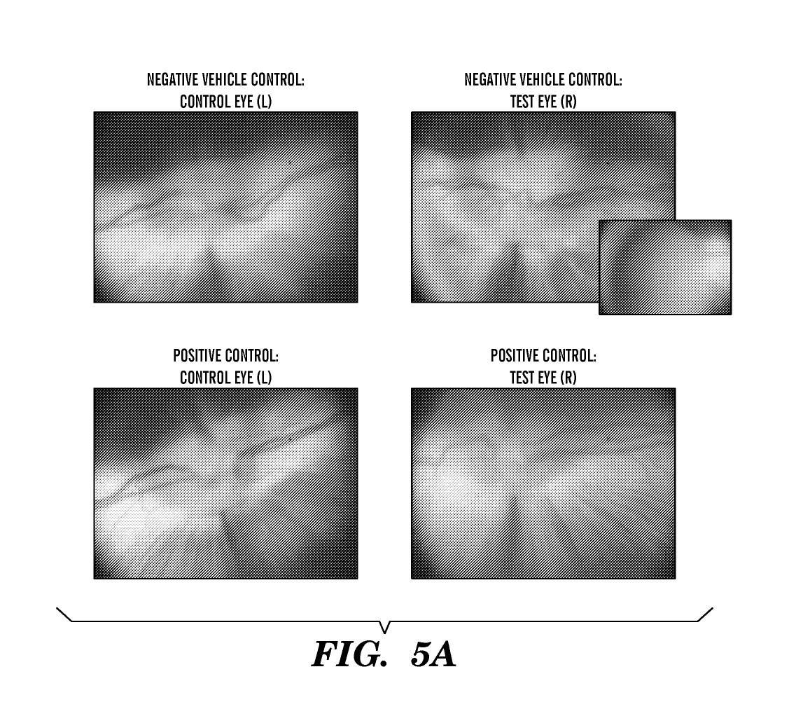

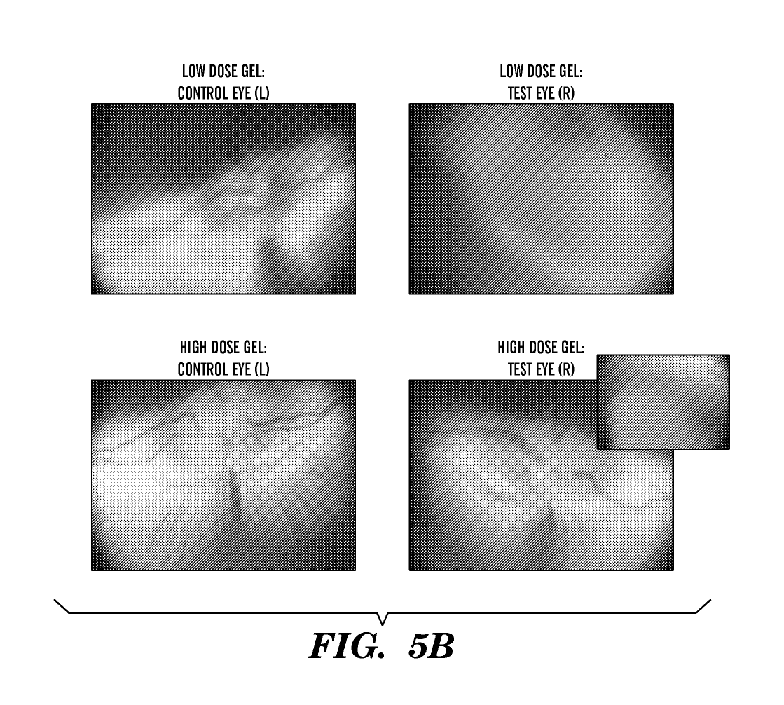

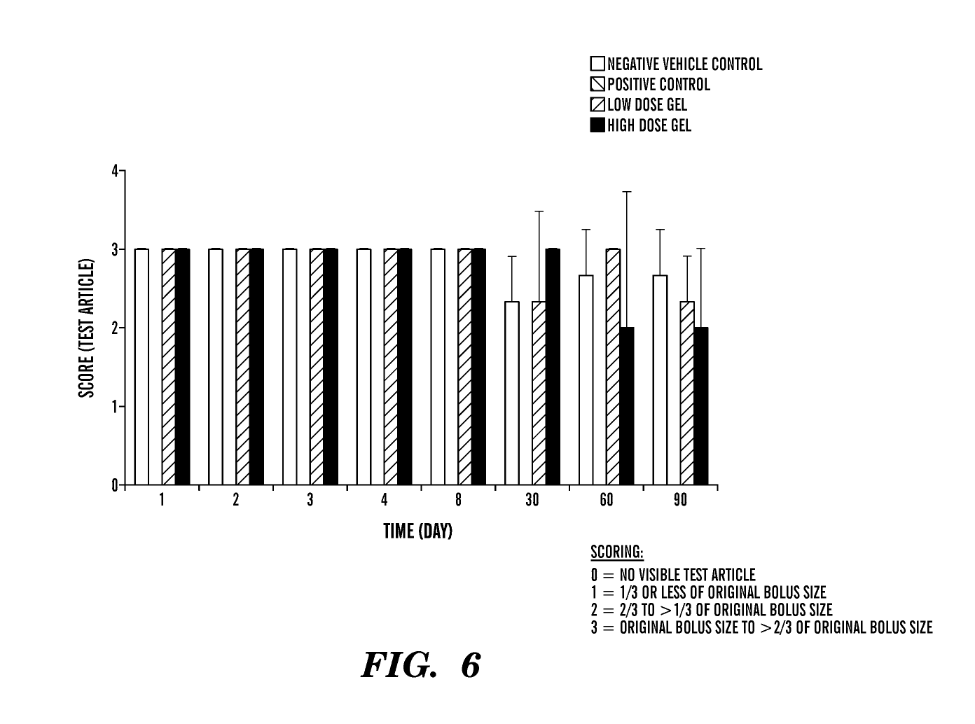

At terminal sacrifice of each group of the rabbits, a series of the fundus photos were taken in order to evaluate the retina, optic disc, and optic nerve for any abnormalities associated with the controls (i.e., no injections) or test formulations (negative control, positive control, “low dose” gel, and “high dose” gel) over the 90-day period. Based on the fundus photos taken over the 90 day period, no abnormality associated with treatment of rabbits was detected in retina, optic disc or optic nerve (see FIGS. 5A-5B). Additionally, degradation of silk hydrogel was visually evaluated over the 90 day period. Up to Day 8 post-dose, little to no degradation of silk hydrogels (e.g., at least ⅔ or more of the original silk hydrogel size remaining) was detected. While biodegradation was detected at later time points (e.g., Day 30 post-dose or later), the remaining silk hydrogel was still greater than ⅓ of the original silk hydrogel size (e.g., between ⅓ to ⅔ of the original silk hydrogel size) even at Day 90 post-dose (see FIG. 6). This visual evaluation was further confirmed at sacrifice as silk hydrogels were more difficult to extract from the vitreous humor at the later time points (e.g., Day 30 post-dose).

### Example 2

### In Vitro Pharmacokinetic Evaluation of Bevacizumab-Loaded Silk Hydrogel Formulations

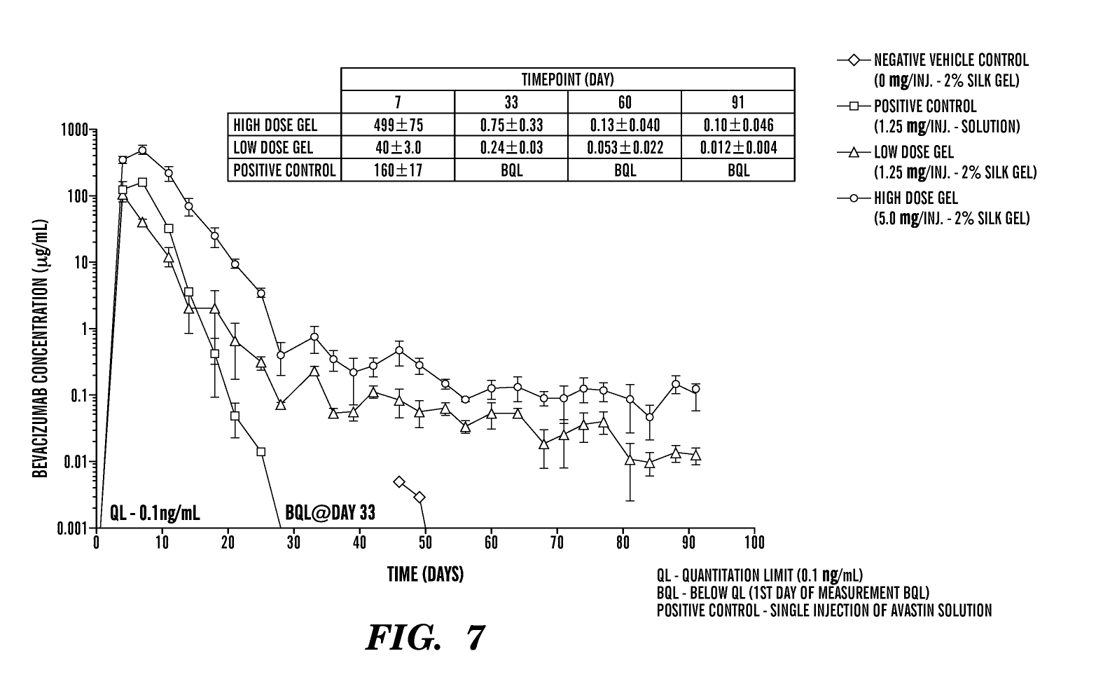

In parallel with the animal study, an in vitro release study was conducted using the same test formulations used to inject the rabbits. Namely, 50 μL of one of the four test formulations: (i) negative vehicle control (i.e., silk hydrogel (2% silk) without therapeutic agent), (ii) positive control (i.e., 2.5% bevacizumab solution (1.25 mg bevacizumab)), (iii) “low dose” silk hydrogel (i.e., 2.5% (w/v) bevacizumab in 2% silk hydrogel (1.25 mg bevacizumab)), and (iv) “high dose” silk hydrogel (i.e., 10% (w/v) bevacizumab in 2% silk hydrogel (5 mg bevacizumab)) was injected into 4 mL of PBS with 0.02% (w/v) sodium azide, with release medium sampled (3.6 mL/sample) and replaced approximately every 3-4 days. Samples were collected and analyzed as described above over the course of 91 days. Similar to the vitreous humor samples in the rabbit study, an initial burst release was detected in the positive control, “low dose” silk hydrogel and “high dose” silk hydrogel, but sustained release was only achieved with the silk hydrogel formulations (see FIG. 7). The positive control was below the detection limit after 30 days of release whereas both “low dose” hydrogel and “high dose” hydrogel exhibited a bevacizumab concentration of approximately 10 ng/mL and 100 ng/mL, respectively, at Day 91 (see FIG. 7). Though the in vitro experiment was stopped at Day 91, both the “low dose” silk hydrogels and “high dose” silk hydrogels had released only approximately 40% and 62%, respectively, of their initial bevacizumab loading. Accordingly, sustained release can be achieved for longer than 3 months with both these silk hydrogel formulations.

With the “low dose” and “high dose” silk hydrogels, bevacizumab concentrations in the vitreous humor at the 90 day time point were equivalent to those levels for the positive solution control at 1 month (˜2 μg/mL). In particular embodiments, silk hydrogels can be used to encapsulate an anti-VEGF agent, e.g., bevacizumab, for sustained release over at least 3 months or longer. Other anti-VEGF therapeutics other than bevacizumab including, but not limited to, ranibizumab (LUCENTIS®, Genentech), aflibercept (VEGF-Trap, Regeneron), and pegaptanib (MACUGEN®, Eyetech) can also be used in some embodiments of the silk hydrogel compositions and/or methods described herein. Some embodiments of the compositions and/or methods described herein can have broad applicability to antibody, peptide, small molecule, and/or RNAi therapeutics and thus can be used for the treatment of a wide range of diseases beyond ocular diseases or disorders such as age-related macular degeneration. In these embodiments, different composition parameters, e.g., low molecular weight silks, different silk concentrations, and/or different silk-to-drug ratios, can be adjusted for release kinetics suitable for different therapeutics and/or treatment of different disease or disorders.

### Example 3

### Exemplary Alternative Embodiments

Anti-VEGF Agent-Loaded Silk Hydrogels:

A range of anti-VEGF agent-loaded silk hydrogel formulations (e.g., bevacizumab-loaded silk hydrogel formulations) were assessed including gels of different silk concentrations/molecular weights, gels containing additives (e.g., PEG, BSA, Tween-20) and lyophilized gels. Silk gel concentrations ranged from 0.5 to 4% (w/v) and an anti-VEGF agent (e.g., bevacizumab) concentrations ranged from 0.4 to 16.7% (w/v). Drug-loaded silk hydrogels were produced by mixing the different silk and drug solutions (e.g., bevacizumab solution) at varied concentrations and ratios before sonication to induce gelation. In some embodiments, lower loading concentrations (<1%) of the anti-VEGF agent (e.g., bevacizumab) in silk gels could result in incomplete release of the loaded drug in vitro, depending on the concentration of silk in the gel formulation (e.g., 23% release from 4% silk/0.4% bevacizumab gel). In alternative embodiments, higher loading concentrations (≧10%) in 2% silk gels could result in greater overall release in vitro, with 70-80% released over the first 10 days and more sustained release over time (≧˜100 ng/day from day 30 to day 60). The rate of release dropped to ng/day levels when approaching 90 days. Depending on the formulation conditions, initial burst release ranged from 3 to 63% with release rates ranging from 0 to 80 ng/day at approximately 30 days of in vitro release.

Anti-VEGF Agent-Loaded Silk Micro/Nanospheres:

Different silk micro/nanospheres can be produced by any methods known in the art. In one embodiment, polyvinyl alcohol (PVA) phase separation was used to produce anti-VEGF agent-loaded silk micro/nanospheres. Briefly, the anti-VEGF agent-loaded silk micro/nanospheres can be produced by: (a) mixing an aqueous silk solution with an aqueous PVA solution; (b) drying the solution mixture, e.g., to form a film; (c) dissolving the dried solid-state silk/PVA blend in water; and (d) removing at least a portion of the PVA, e.g., by centrifuging to remove the residual PVA. See, e.g., International Application No. WO 2011/041395, the content of which is incorporated herein by reference, for additional details of the PVA phase separation method for production of silk microspheres. Different anti-VEGF agent (e.g., bevacizumab) loading conditions were employed from pre-loading an anti-VEGF agent (mixing silk with anti-VEGF agent, e.g., bevacizumab prior to sphere formation) to post-loading an anti-VEGF agent (loading spheres with an anti-VEGF agent, e.g., bevacizumab after formation). Spheres were post-loaded, e.g., by suspending spheres in a desired volume of an anti-VEGF agent (e.g., bevacizumab) solution (˜0.1 to ˜25%) before lyophilizing directly to achieve the final loading. In some embodiments, the spheres were post-loaded by incubating the spheres in an anti-VEGF solution (e.g., for ˜0.5-˜2 hours) before concentrating the anti-VEGF solution and spheres, e.g., using a centrifugal filtration unit, and incubating again (e.g., ˜0.5-˜2 hours), and repeating this process as desired before lyophilizing to yield the final sphere formulation. Spheres can also be prepared using a combination of pre- and post-loading techniques. By way of example only, bevacizumab loading was performed, e.g., using either stock bevacizumab (25 mg/mL) or concentrated bevacizumab (˜200 mg/mL), with final loading ranging from 0.014 mg bevacizumab/mg silk to 0.553 mg bevacizumab/mg silk. Sphere formulations generally exhibited burst release kinetics, releasing 60-100% of drug within 3-4 days. Silk/PVA blend ratios could range from about 1/6 to about 1/2. In one embodiment, the silk/PVA blend ratio was typically about 1/4, with component concentrations typically about 5% silk and about 5% PVA. While increasing the silk/PVA blend ratios up to 1/2 could result in less homogeneous size distribution of silk microspheres, such silk/PVA blend ratios had no significant influence on drug release kinetics. The burst release kinetics can be adjusted, e.g., by varying the ratio of silk solution to PVA solution. In some embodiments, concentrated anti-VEGF agent (e.g., bevacizumab) was used to formulate pre-, post-, and pre/post-loaded silk spheres. In these embodiments, the bevacizumab-loaded silk spheres exhibited in vitro release of approximately 1.4 mg/day/10 mg of spheres for the first 3 days and concentrations of (3-5 μg/day) for 10-14 days with overall release up to 3-4 weeks. Depending on the formulation conditions and/or the molecular size of the anti-VEGF agent, initial burst release, e.g., of bevacizumab, ranged from 6 to 100% with release rates ranging from about 0 to about 1000 μg/day, or from about 0 to about 100 μg/day, or from about 0 to about 400 ng/day over a period of approximately 30 days of in vitro release.

Anti-VEGF Agent-Loaded Silk Micro/Nanospheres in Silk Gels:

PVA nanospheres loaded with an anti-VEGF agent (e.g., bevacizumab) were mixed into silk hydrogels. See, e.g., International Application No. WO 2010/141133, the content of which is incorporated herein by reference, for additional details on production of antibiotic-loaded silk microspheres embedded in a silk scaffold. The nanosphere formulations included pre-, post-, and pre/post-loaded with bevacizumab while the silk hydrogels were, e.g., either 1 or 0.5% silk and loaded with 2% or 2.3% anti-VEGF agent (e.g., bevacizumab), respectively. Bevacizumab-loaded microspheres were prepared from silk (e.g., ˜6-˜8%) and bevacizumab (e.g., ˜2.5%) at a ratio of approximately 1/4 (w/w) bevacizumab/silk before being embedded into silk hydrogels (e.g., ˜1 or ˜0.5% silk). Without wishing to be bound by theory, in general, higher concentration silk gels (e.g., 1% vs. 0.5% silk) reduced the overall percent release (e.g., ˜50-80% (1% silk) vs. ˜60-100% (0.5% silk) at day 32), while the addition of silk microspheres loaded with higher amounts of an anti-VEGF agent (e.g., bevacizumab), for example, by mixing the anti-VEGF agent into the silk solution before microsphere formation and further loading the formed microspheres with the anti-VEGF agent after microsphere formation (designated herein as “pre/post-loaded spheres”), improved the overall release. In some embodiments, the bevacizumab-loaded microspheres (e.g., approximately 1/4 ratio (w/w) bevacizumab/silk prepared from 2.5% bevacizumab and 6-8% silk blended with PVA at a ratio of 1/4 with component concentrations of ˜5% bevacizumab/silk and 5% PVA) in a bevacizumab-loaded silk hydrogel (e.g., ˜1% bevacizumab in 1% silk hydrogel) exhibited in vitro release of approximately 200-450 μg/day/100 μL for the first 7 days, concentrations of 10-20 μg/day for up to 14 days and overall release up to 1 month for a 1% silk/3% bevacizumab formulation (bevacizumab-loaded gel+pre/post-loaded spheres). Depending on the formulation conditions and/or the molecular size of the anti-VEGF agent, initial burst release, e.g., of bevacizumab, ranged from 21 to 50% with release rates ranging from about 0 to about 1000 μg/day, or from about 0 to about 100 μg/day or from about 0 to about 100 ng/day over a period of approximately 30 days of in vitro release.

Therapeutic Agent-Loaded Gel or Gel-Like Particles:

In some embodiments, the therapeutic agent-loaded gel or gel-like particles can be produced from a silk hydrogel. A silk hydrogel can be produced by any methods known in the art. In one embodiment, to prepare a silk hydrogel, a regenerated aqueous solution of silk fibroin at a silk concentration between approx. 8 wt. % and approx. 30 wt. % was mixed with an aqueous solution or dispersion containing the therapeutic agent to obtain mass ratios of silk to the therapeutic agent between 0.1 to 1000. The silk-therapeutic agent mixture was sonicated using a Branson Sonifier (Branson Ultrasonics Corp., Danbury, Conn.) at a sonication power and duration that depended on the silk and drug solution concentration and solution volume. See additional details on silk hydrogel preparation, e.g., in X., Wang, J., Kluge, G., Leisk, D. L., Kaplan. Sonication control of silk gelation for cell delivery systems. Biomaterials. 2008; 29: 1054-1064 and International App. No. WO 2008/150861. Sonicated silk-drug solutions (or sols) were incubated at 37° C. for a desired duration (from hours to weeks) until complete hydrogelation.

Sonicated silk-therapeutic agent hydrogels were prepared into micrometer-sized hydrogel particles, or micro-gels using any known methods in the art, e.g., cutting or crushing. In one embodiment, the sonicated silk-therapeutic agent hydrogels were prepared into micrometer-sized hydrogel particles using a graduated series of metal sieves with desired pore sizes (stainless steel woven wire cloth, McMaster-Carr, pore size ranging from 30 μm up to millimeters). The hydrogel was pressed through this series of metal sieves using a spatula into plastic petri dishes to form the micro-gels. If necessary, the hydrogel was repeatedly pressed through metal sieves having smaller mesh sizes until the desired micro-gel size distribution was obtained.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A composition for ocular administration comprising a therapeutic agent encapsulated in a silk matrix, wherein an amount of the therapeutic agent encapsulated in the silk matrix provides a therapeutic effect for a period of time which is longer than when the same amount of the therapeutic agent is administered without the silk matrix.

2. The composition of claim 1, wherein the therapeutic effect comprises a therapeutic effect for treatment of an ocular condition.

3. The composition of claim 2, wherein the therapeutic effect for treatment of the ocular condition includes a reduction of at least one symptom associated with the ocular condition by at least about 10%.

4. The composition of any of claims 1-3, wherein the period of time is at least about 1 week longer than when the same amount of the therapeutic agent is administered without the silk matrix.

5. The composition of any of claims 1-4, wherein the period of time is at least about 1 month, at least about 3 months, or at least about 6 months longer than when the same amount of the therapeutic agent is administered without the silk matrix.

6. The composition of any of claims 1-5, wherein the therapeutic agent is selected from the group consisting of proteins, peptides, antigens, immunogens, vaccines, antibodies or portions thereof, antibody-like molecules, enzymes, nucleic acids, siRNA, shRNA, aptamers, small molecules, antibiotics, and any combinations thereof.

7. The composition of any of claims 1-6, wherein the therapeutic agent is an agent for treatment of an ocular condition.

8. The composition of any of claims 1-7, wherein the therapeutic agent is selected from the group consisting of bevacizumab, ranibizumab, aflibercept, pegaptanib, tivozanib, fluocinolone acetonide, ganciclovir, triamcinolone acetonide, foscarnet, vancomycin, ceftazidime, amikacin, amphotericin B, dexamethasone, and any combinations thereof.

9. The composition of any of claims 1-8, wherein the therapeutic agent comprises an angiogenesis inhibitor.

10. The composition of claim 9, wherein the angiogenesis inhibitor comprises a VEGF inhibitor.

11. The composition of claim 10, wherein the VEGF inhibitor is selected from the group consisting of bevacizumab, ranibizumab, aflibercept, pegaptanib, tivozanib, 3-(4-Bromo-2,6-difluoro-benzyloxy)-5-[3-(4-pyrrolidin 1-yl-butyl)-ureido]-isothiazole-4-carboxylic acid amide hydrochloride, axitinib, N-(4-bromo-2-fluorophenyl)-6-methoxy-7-[(1-methylpiperidin-4-yl)methoxy]quinazolin-4-amine, an inhibitor of VEGF-R2 and VEGF-R1, axitinib, N,2-dimethyl-6-(2-(1-methyl-1H-imidazol-2-yl)thieno[3,2-b]pyridin-7-yloxy)benzo[b]thiophene-3-carboxamide, tyrosine kinase inhibitor of the RET/PTC oncogenic kinase, N-(4-bromo-2-fluorophenyl)-6-methoxy-7-[(1-methylpiperidin-4-yl) methoxy]quinazolin-4-amine, pan-VEGF-R-kinase inhibitor; protein kinase inhibitor, multitargeted human epidermal receptor (HER) 1/2 and vascular endothelial growth factor receptor (VEGFR) 1/2 receptor family tyrosine kinases inhibitor, cediranib, sorafenib, vatalanib, glufanide disodium, VEGFR2-selective monoclonal antibody, angiozyme, an siRNA-based VEGFR1 inhibitor, Fumagillin and analogue thereof, soluble ectodomains of the VEGF receptors, shark cartilage and derivatives thereof, 5-((7-Benzyloxyquinazolin-4-yl)amino)-4-fluoro-2-methyl phenol hydrochloride, any derivatives thereof and any combinations thereof.

12. The composition of claim 10 or 11, wherein the VEGF inhibitor is bevacizumab, ranibizumab, or a combination thereof.

13. The composition of any of claims 1-12, wherein the therapeutic agent or the VEGF inhibitor is present in an amount of about 0.01 mg to about 50 mg.

14. The composition of any of claims 1-13, wherein the therapeutic agent or the VEGF inhibitor is present in an amount of about 1.5 mg to about 10 mg, or about 5 mg to about 10 mg.

15. The composition of any of claims 1-14, wherein the silk matrix comprises silk fibroin at a concentration of about 0.1% (w/v) to about 50% (w/v).

16. The composition of any of claims 1-15, wherein the silk matrix comprises silk fibroin at a concentration of about 0.5% (w/v) to about 30% (w/v).

17. The composition of any of claims 1-16, wherein the silk matrix comprises silk fibroin at a concentration of about 1% (w/v) to about 15% (w/v).

18. The composition of any of claims 1-17, wherein the silk matrix further comprises a biocompatible polymer.

19. The composition of claim 18, wherein the biocompatible polymer is selected from the group consisting of a poly-lactic acid (PLA), poly-glycolic acid (PGA), poly-lactide-co-glycolide (PLGA), polyesters, poly(ortho ester), poly(phosphazine), poly(phosphate ester), polycaprolactone, gelatin, collagen, cellulose, hyaluronan, poly(ethylene glycol) (PEG), triblock copolymers, polylysine and any derivatives thereof.

20. The composition of any of claims 1-19, wherein the silk matrix is selected from the group consisting of hydrogel, microparticle, nanoparticle, fiber, film, lyophilized powder, lyophilized gel, reservoir implant, homogenous implant, tube, gel-like or gel particle, and any combinations thereof.

21. The composition of any of claims 1-20, wherein the silk matrix comprises a hydrogel.

22. The composition of any of claims 1-20, wherein the silk matrix comprises a microparticle, a nanoparticle, or a gel-like or gel particle.

23. The composition of claim 22, wherein the microparticle, the nanoparticle, or the gel-like or gel particle encapsulating the therapeutic agent is embedded in a solid substrate.

24. The composition of claim 23, wherein the solid substrate is selected from the group consisting of a tablet, a capsule, a microchip, a hydrogel, a mat, a film, a fiber, an ocular delivery device, an implant, a tube, a coating, and any combinations thereof.

25. The composition of claim 23 or 24, wherein the solid substrate comprises a hydrogel.

26. The composition of claim 25, wherein the hydrogel comprises a silk hydrogel.

27. The composition of any of claims 1-26, wherein the composition is adapted to be injectable.

28. The composition of claim 27, wherein the composition is pre-loaded into a syringe.

29. The composition of claim 28, wherein the syringe is further attached to an injection needle.

30. The composition of any of claims 1-29, wherein the ocular administration is administration of the composition to at least a portion of an eye selected from the group consisting of lens, sclera, conjunctiva, aqueous humor, ciliary muscle, and vitreous humor.

31. The composition of any of claims 1-30, wherein the ocular administration is intravitreal administration.

32. An ocular delivery device comprising the composition of any of claims 1-31.

33. The ocular delivery device of claim 32, wherein the composition is pre-loaded into the ocular delivery device.

34. The ocular delivery device of claim 32, wherein the device is a syringe with or without an injection needle.

35. The ocular delivery device of claim 34, wherein the injection needle is a 25- to 34-gauge needle.

36. The ocular delivery device of claim 34 or 35, wherein the injection needle is a 27- to 30-gauge needle.

37. The ocular delivery device of claim 32, wherein the device comprises a contact lens.

38. The ocular delivery device of claim 32, wherein the device comprises an eye-dropper.

39. The ocular delivery device of claim 32, wherein the device comprises a microneedle.

40. The ocular delivery device of claim 39, wherein the microneedle is a silk microneedle.

41. The ocular delivery device of claim 32, wherein the device is an implant.

42. A kit comprising a container containing a composition of any of claims 1-31, or an ocular delivery device of any of claims of 32-41.

43. The kit of claim 42, further comprising at least a syringe and an injection needle.

44. The kit of claim 43, wherein the injection needle is a 25- to 34-gauge needle.

45. The kit of claim 43 or 44, wherein the injection needle is a 27- to 30-gauge needle.

46. The kit of any of claims 42-45, further comprising an anesthetic.

47. The kit of any of claims 42-46, further comprising an antiseptic agent.

48. The kit of any of claims 42-47, wherein the ocular delivery device is pre-loaded with the composition.

49. The kit of claim 48, wherein the ocular delivery device is a syringe with or without an injection needle.

50. A method for delivering a therapeutic agent to a target site of an eye comprising administering to a target site of an eye a therapeutic agent encapsulated in a silk matrix, wherein an amount of the therapeutic agent encapsulated in the silk matrix provides a therapeutic effect for a period of time which is longer than when the same amount of the therapeutic agent is administered without the silk matrix.

51. A method for treating an ocular condition in a subject comprising administering to target site of an eye of a subject a composition of any of claims 1-29, thereby treating the ocular condition with a sustained release of the therapeutic agent to the target site of the eye.

52. The method of claim 51, wherein the ocular condition is a condition of a posterior segment of the eye.

53. The method of claim 51 or 52, wherein the ocular condition is selected from the group consisting of age-related macular degeneration, choroidal neovascularization, diabetic macular edema, acute and chronic macular neuroretinopathy, central serous chorioretinopathy, macular edema, acute multifocal placoid pigment epitheliopathy, Behcet's disease, birdshot retinochoroidopathy, posterior uveitis, posterior scleritis, serpignous choroiditis, subretinal fibrosis, uveitis syndrome, Vogt-Koyanagi-Harada syndrome, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, carotid artery disease (CAD), frosted branch angitis, sickle cell retinopathy, angioid streaks, familial exudative vitreoretinopathy, Eales disease, proliferative vitreal retinopathy, diabetic retinopathy, retinal disease associated with tumors, congenital hypertrophy of the retinal pigment epithelium (RPE), posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors, myopic retinal degeneration, acute retinal pigment epithelitis, glaucoma, endophthalmitis, cytomegalovirus retinitis, retinal cancers and any combinations thereof.

54. The method of claim 53, wherein the ocular condition is age-related macular degeneration.

55. The method of claim 53, wherein the therapeutic agent comprises a VEGF inhibitor.

56. The method of claim 55, wherein the VEGF inhibitor comprises bevacizumab, ranibizumab, or a combination thereof.

57. A method for administrating a therapeutic agent to a target site of an eye of a subject in need thereof comprising administrating to a target site of an eye of a subject the composition of any of claims 1-31 at an administration frequency less than when the same amount of the therapeutic agent is administered without the silk matrix.

58. The method of claim 57, wherein the administration frequency is reduced by a factor of ½.

59. A method for increasing an effective amount of a therapeutic agent administered to an eye comprising administering to a target site of an eye a therapeutic agent encapsulated in a silk matrix, wherein upon administration, leakage of the therapeutic agent from the target site is reduced, as compared to when the same amount of the therapeutic agent is administered without the silk matrix, thereby increasing the effective amount of the therapeutic agent administered to the target site of the eye.

60. The method of any of claims 50-59, wherein the therapeutic agent encapsulated in the silk matrix or the composition is administered to the anterior segment of the eye.

61. The method of any of claims 50-60, wherein the therapeutic agent encapsulated in the silk matrix or the composition is administered to the posterior segment of the eye.

62. The method of any of claims 50-61, wherein the therapeutic agent encapsulated in the silk matrix or the composition is administered to at least a portion of the eye selected from the group consisting of lens, sclera, conjunctiva, aqueous humor, ciliary muscle, and vitreous humor.

63. The method of any of claims 50-62, wherein the therapeutic agent encapsulated in the silk matrix or the composition is administered to the vitreous humor of the eye.

64. The method of any of claims 50-63, wherein the therapeutic agent encapsulated in the silk matrix or the composition is administered to the eye by injection.

65. The method of claim 64, wherein the injection is performed with a needle with a gauge of about 25 to about 34.

66. Then method claim 64 or 65, wherein the injection is performed with a needle with a gauge of about 27 to about 30.

67. The method of any of claims 50-66, wherein the administration is performed no more than once a month.

68. The method of any of claims 50-67, wherein the administration is performed no more than once every two months.

69. The method of any of claims 50-68, wherein the administration is performed no more than once every three months, no more than once every four months or no more once every six months.

70. The method of any of claims 50-69, wherein the therapeutic agent is selected from the group consisting of proteins, peptides, antigens, immunogens, vaccines, antibodies or portions thereof, antibody-like molecules, enzymes, nucleic acids, siRNA, shRNA, aptamers, small molecules, antibiotics, and any combinations thereof.

71. The method of any of claims 50-70, wherein the therapeutic agent is selected from the group consisting of bevacizumab, ranibizumab, aflibercept, pegaptanib, tivozanib, fluocinolone acetonide, ganciclovir, triamcinolone acetonide, foscarnet, vancomycin, ceftazidime, amikacin, amphotericin B, dexamethasone, and any combinations thereof.

72. The method of any of claims 50-71, wherein the therapeutic agent comprises an angiogenesis inhibitor.

73. The method of any of claims 50-72, wherein the angiogenesis inhibitor comprises a VEGF inhibitor.

74. The method of claim 73, wherein the VEGF inhibitor is selected from the group consisting of bevacizumab, ranibizumab, aflibercept, pegaptanib, tivozanib, 3-(4-Bromo-2,6-difluoro-benzyloxy)-5-[3-(4-pyrrolidin 1-yl-butyl)-ureido]-isothiazole-4-carboxylic acid amide hydrochloride, axitinib, N-(4-bromo-2-fluorophenyl)-6-methoxy-7-[(1-methylpiperidin-4-yl)methoxy]quinazolin-4-amine, an inhibitor of VEGF-R2 and VEGF-R1, axitinib, N,2-dimethyl-6-(2-(1-methyl-1H-imidazol-2-yl)thieno[3,2-b]pyridin-7-yloxy)benzo[b]thiophene-3-carboxamide, tyrosine kinase inhibitor of the RET/PTC oncogenic kinase, N-(4-bromo-2-fluorophenyl)-6-methoxy-7-[(1-methylpiperidin-4-yl) methoxy]quinazolin-4-amine, pan-VEGF-R-kinase inhibitor; protein kinase inhibitor, multitargeted human epidermal receptor (HER) 1/2 and vascular endothelial growth factor receptor (VEGFR) 1/2 receptor family tyrosine kinases inhibitor, cediranib, sorafenib, vatalanib, glufanide disodium, VEGFR2-selective monoclonal antibody, angiozyme, an siRNA-based VEGFR1 inhibitor, Fumagillin and analogue thereof, soluble ectodomains of the VEGF receptors, shark cartilage and derivatives thereof, 5-((7-Benzyloxyquinazolin-4-yl)amino)-4-fluoro-2-methyl phenol hydrochloride, any derivatives thereof and any combinations thereof.

75. The method of claim 74, wherein the VEGF inhibitor comprises bevacizumab, ranibizumab, or a combination thereof.

76. The method of any of claims 50-75, wherein the therapeutic agent or the VEGF inhibitor is present in an amount of about 0.01 mg to about 50 mg.

77. The method of any of claims 50-76, wherein the therapeutic agent or the VEGF inhibitor is present in an amount of about 1.5 mg to about 10 mg, or about 5 mg to about 10 mg.

78. The method of any of claims 50-77, wherein the silk matrix comprises silk fibroin at a concentration of about 0.1% (w/v) to about 50% (w/v).

79. The method of any of claims 50-78, wherein the silk matrix comprises silk fibroin at a concentration of about 0.5% (w/v) to about 30% (w/v).

80. The method of any of claims 50-79, wherein the silk matrix comprises silk fibroin at a concentration of about 1% (w/v) to about 15% (w/v).

81. The method of any of claims 50-80, wherein the silk matrix further comprises a biocompatible polymer.

82. The method of claim 81, wherein the biocompatible polymer is selected from the group consisting of a poly-lactic acid (PLA), poly-glycolic acid (PGA), poly-lactide-co-glycolide (PLGA), polyesters, poly(ortho ester), poly(phosphazine), poly(phosphate ester), polycaprolactone, gelatin, collagen, cellulose, hyaluronan, poly(ethylene glycol) (PEG), triblock copolymers, polylysine and any derivatives thereof.

83. The method of any of claims 50-82, wherein the silk matrix is selected from the group consisting of hydrogel, microparticle, nanoparticle, fiber, film, lyophilized powder, lyophilized gel, reservoir implant, homogenous implant, a tube, gel-like or gel particle, and any combinations thereof.

84. The method of any of claims 50-83, wherein the silk matrix comprises a hydrogel.

85. The method of any of claims 50-84, wherein the silk matrix comprises a microparticle, a nanoparticle, or a gel-like or gel particle.

86. The method of claim 85, wherein the microparticle, the nanoparticle, or the gel-like or gel particle encapsulating the therapeutic agent is embedded in a solid substrate.

87. The method of claim 86, wherein the solid substrate is selected from the group consisting of a tablet, a capsule, a microchip, a hydrogel, a mat, a film, a fiber, an ocular delivery device, an implant, a tube, a coating, and any combinations thereof.

88. The method of claim 86 or 87, wherein the solid substrate comprises a hydrogel.

89. The method of claim 88, wherein the hydrogel comprises a silk hydrogel.

90. The method of any of claims 50-89, wherein the therapeutic agent is released from the silk matrix at a rate such that at least about 20% of the therapeutic agent initially encapsulated in the silk matrix is released over a period of at least about 3 months.

91. The method of any of claims 50-90, wherein the therapeutic agent is released from the silk matrix at the rate such that at least about 40% of the therapeutic agent initially encapsulated in the silk matrix is released over a period of at least about 3 months.

92. The method of any of claims 50-91, wherein the therapeutic agent is released from the silk matrix at the rate such that at least about 60% of the therapeutic agent initially encapsulated in the silk matrix is released over a period of at least about 3 months.

93. The method of any of claims 50-92, wherein the therapeutic agent is released from the silk matrix at the rate of about 1 ng/day to about 15 mg/day.

94. The method of any of claims 50-93, wherein the therapeutic agent is released from the silk matrix at the rate of about 1 μg/day to about 1 mg/day.

95. The method of any of claims 50-94, wherein the therapeutic effect provided by the amount of the therapeutic agent encapsulated in the silk matrix comprises a therapeutic effect for treatment of an ocular condition.

96. The method of claim 95, wherein the therapeutic effect for treatment of the ocular condition comprises a reduction of at least one symptom associated with the ocular condition by at least about 10%.

97. The method of any of claims 50-96, wherein the period of time of the therapeutic effect provided by the amount of the therapeutic agent encapsulated in the silk matrix is at least about 1 week longer than when the same amount of the therapeutic agent is administered without the silk matrix.

98. The method of any of claims 50-97, wherein the period of time of the therapeutic effect provided by the amount of the therapeutic agent encapsulated in the silk matrix is at least about 1 month longer than when the same amount of the therapeutic agent is administered without the silk matrix.

99. The method of any of claims 50-98, wherein the period of time of the therapeutic effect provided by the amount of the therapeutic agent encapsulated in the silk matrix is at least about 3 months longer than when the same amount of the therapeutic agent is administered without the silk matrix.

100. The method of any of claims 50-99, wherein the period of time of the therapeutic effect provided by the amount of the therapeutic agent encapsulated in the silk matrix is at least about 6 months longer than when the same amount of the therapeutic agent is administered without the silk matrix.

101. A method of producing a controlled-release silk-based composition for ocular administration comprising contacting with water vapor a silk-based matrix, the silk matrix comprising at least one therapeutic agent encapsulated therein.

102. The method of claim 101, wherein the silk-based matrix to be contacted with the water vapor is a non-crosslinked silk-based matrix.

103. The method of claim 101 or 102, wherein the contact of the silk-based matrix with water vapor induces formation of beta sheet structures in silk fibroin.

104. The method of any of claims 101-103, wherein the contact of the silk-based matrix with the water vapor modulates release kinetics of said at least one therapeutic agent from the silk-based matrix.

105. The method of any of claims 101-104, wherein said at least one therapeutic agent is selected from the group consisting of proteins, peptides, antigens, immunogens, vaccines, antibodies or portions thereof, antibody-like molecules, enzymes, nucleic acids, siRNA, shRNA, aptamers, small molecules, antibiotics, and any combinations thereof.

106. The method of any of claims 101-105, wherein the therapeutic agent is selected from the group consisting of bevacizumab, ranibizumab, aflibercept, pegaptanib, tivozanib, fluocinolone acetonide, ganciclovir, triamcinolone acetonide, foscarnet, vancomycin, ceftazidime, amikacin, amphotericin B, dexamethasone, and any combinations thereof.

107. The method of any of claims 101-106, wherein the therapeutic agent comprises an angiogenesis inhibitor.

108. The method of claim 107, wherein the angiogenesis inhibitor comprises a VEGF inhibitor.

109. The method of claim 108, wherein the VEGF inhibitor is selected from the group consisting of bevacizumab, ranibizumab, aflibercept, pegaptanib, tivozanib, 3-(4-Bromo-2,6-difluoro-benzyloxy)-5-[3-(4-pyrrolidin 1-yl-butyl)-ureido]-isothiazole-4-carboxylic acid amide hydrochloride, axitinib, N-(4-bromo-2-fluorophenyl)-6-methoxy-7-[(1-methylpiperidin-4-yl)methoxy]quinazolin-4-amine, an inhibitor of VEGF-R2 and VEGF-R1, axitinib, N,2-dimethyl-6-(2-(1-methyl-1H-imidazol-2-yl)thieno[3,2-b]pyridin-7-yloxy)benzo[b]thiophene-3-carboxamide, tyrosine kinase inhibitor of the RET/PTC oncogenic kinase, N-(4-bromo-2-fluorophenyl)-6-methoxy-7-[(1-methylpiperidin-4-yl) methoxy]quinazolin-4-amine, pan-VEGF-R-kinase inhibitor; protein kinase inhibitor, multitargeted human epidermal receptor (HER) 1/2 and vascular endothelial growth factor receptor (VEGFR) 1/2 receptor family tyrosine kinases inhibitor, cediranib, sorafenib, vatalanib, glufanide disodium, VEGFR2-selective monoclonal antibody, angiozyme, an siRNA-based VEGFR1 inhibitor, Fumagillin and analogue thereof, soluble ectodomains of the VEGF receptors, shark cartilage and derivatives thereof, 5-((7-Benzyloxyquinazolin-4-yl)amino)-4-fluoro-2-methyl phenol hydrochloride, any derivatives thereof and any combinations thereof.

110. The method of claim 108, wherein the VEGF inhibitor comprises bevacizumab, ranibizumab, or a combination thereof.

111. The method of any of claims 101-110, wherein the silk matrix is selected from the group consisting of hydrogel, microparticle, nanoparticle, fiber, film, lyophilized powder, lyophilized gel, reservoir implant, homogenous implant, a tube, gel-like or gel particle, and any combinations thereof.

112. The method of any of claims 101-111, wherein said at least one therapeutic agent is encapsulated in a silk hydrogel.

113. The method of any of claims 101-112, wherein said at least one therapeutic agent is encapsulated in silk microparticles, silk nanoparticles, gel-like or gel particles, or any combinations thereof.

114. The method of claim 113, the silk microparticles, silk nanoparticles, gel-like or gel particles encapsulating said at least one therapeutic agent are further embedded in a hydrogel.

115. The method of claim 114, wherein the hydrogel comprises a silk hydrogel.
